(19) United States
(12) Patent Application Publication
(10) Pub. No.: US 2011/0021487 A1
(43) Pub. Date: Jan. 27, 2011

(54) CYCLOALKOXY-SUBSTITUTED 4-PHENYL-3,5-DICYANOPYRIDINES AND THEIR USE

(75) Inventors: Walter Hübsch, Wuppertal (DE); Peter Nell, Wuppertal (DE); Frank Süssmeier, Wuppertal (DE); Alexandros Vakalopoulos, Hilden (DE); Daniel Meibom, Leverkusen (DE); Barbara Albrecht-Küpper, Wulfrath (DE); Katja Zimmermann, Dusseldorf (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: BAYER SCHERING PHARMA AKTIENGESELLSCHAFT, Berlin (DE)

(21) Appl. No.: 12/867,560

(22) PCT Filed: Jan. 31, 2009
(86) PCT No.: PCT/EP09/00645

§ 371 Date: Aug. 13, 2010

(30) Foreign Application Priority Data
Feb. 13, 2008 (DE) 10 2008 008 838.2

(51) Int. Cl. A61K 31/44; C07D 401/12; C07D 417/12; C07D 213/85; C07D 405/14; C07D 413/14; A61K 31/444; A61K 31/4439; A61P 9/12; A61P 9/00; A61P 9/04; A61P 9/10; A61P 3/00; A61P 3/10; A61P 3/06

(52) U.S. Cl. 514/210.2; 546/261; 546/269.7; 546/270.7; 546/275.1; 546/287; 546/256; 546/271.4; 514/335; 514/342; 514/341; 514/344; 514/333; 514/340

(57) ABSTRACT

The present application relates to novel cycloalkoxy-substituted 4-phenyl-3,5-dicyanopyridine derivatives, to processes for their preparation, to their use for the treatment and/or prevention of diseases and to their use for preparing medicaments for the treatment and/or prevention of diseases, preferably for the treatment and/or prevention of cardiovascular and metabolic disorders.

The present application relates to novel cycloalkoxy-substituted 4-phenyl-3,5-dicyanopyridine derivatives, to processes for their preparation, to their use for the treatment and/or prevention of diseases and to their use for preparing medicaments for the treatment and/or prevention of diseases, preferably for the treatment and/or prevention of cardiovascular and metabolic disorders.

Adenosine, a purine nucleoside, is present in all cells and is released by a large number of physiological and pathophysiological stimuli. Adenosine is formed intracellularly as an intermediate during the degradation of adenosine 5′-monophosphate (AMP) and S-adenosylhomocysteine, but it can be released from the cell, in which case it acts as a hormone-like substance or neurotransmitter by binding to specific receptors.

Under normoxic conditions, the concentration of free adenosine in the extracellular space is very low. However, under ischemic or hypoxic conditions, the extracellular concentration of adenosine in the affected organs is increased dramatically. Thus, it is known, for example, that adenosine inhibits platelet aggregation and increases the blood supply to the coronary arteries. Furthermore, it acts on the blood pressure, on the heart rate, on the release of neurotransmitters and on lymphocyte differentiation. In adipocytes, adenosine is capable of inhibiting lipolysis, thus lowering the concentration of free fatty acids and triglycerides in the blood.

The aim of these actions of adenosine is to increase the oxygen supply of the affected organs and/or to reduce the metabolism of these organs in order to adjust the metabolism of the organ to the blood supply of the organ under ischemic or hypoxic conditions.

The action of adenosine is mediated via specific receptors. To date, subtypes A1, A2a, A2b and A3 are known. According to the invention, “adenosine-receptor-selective ligands” are substances which bind selectively to one or more subtypes of the adenosine receptors, thus either mimicking the action of adenosine (adenosine agonists) or blocking its action (adenosine antagonists).

The actions of these adenosine receptors are mediated intracellularly by the messenger cAMP. In the case of the binding of adenosine to the A2a or A2b receptors, the intracellular cAMP is increased via activation of the membrane-bound adenylate cyclase, whereas binding of adenosine to the A1 or A3 receptors results in a decrease of the intracellular cAMP concentration via inhibition of adenylate cyclase.

In the cardiovascular system, the main consequences of the activation of adenosine receptors are: bradycardia, negative inotropism and protection of the heart against ischemia (“preconditioning”) via A1 receptors, dilation of the blood vessels via A2a and A2b receptors and inhibition of the fibroblasts and smooth-muscle-cell proliferation via A2b receptors.

In the case of A1 agonists (coupling preferably via Gᵢ proteins), a decrease of the intracellular cAMP concentration is observed (preferably after direct prestimulation of adenylate cyclase by forskolin). Correspondingly, A2a and A2b agonists (coupling preferably via Gₛ proteins) leads to an increase and A2a and A2b antagonists to a decrease of the cAMP concentration in the cells. In the case of A2 receptors, a direct prestimulation of adenylate cyclase by forskolin is of no benefit.

In humans, activation of A1 receptors by specific A1 agonists leads to a frequency-dependent lowering of the heart rate, without any effect on blood pressure. Selective A1 agonists may thus be suitable inter alia for treating angina pectoris and atrial fibrillation.

The activation of A2b receptors by adenosine or specific A2b agonists leads, via dilation of blood vessels, to lowering of the blood pressure. The lowering of the blood pressure is accompanied by a reflectory increase in heart rate. The increased heart rate can be reduced by activation of A1 receptors using specific A1 agonists.

The combined action of selective A1/A2b agonists on the vascular system and heart rate thus results in a systemic lowering of the blood pressure without relevant heart-rate increase. Dual A1/A2b agonists having such a pharmacological profile could be employed, for example, for treating hypertension in humans.

In adipocytes, the activation of A1 and A2b receptors leads to an inhibition of lipolysis. Thus, the selective or combined action of A1 and A1/A2b agonists on lipid metabolism results in a lowering of free fatty acids and triglycerides. In turn, in patients suffering from metabolic syndrome and in diabetics, reducing lipids leads to lower insulin resistance and improved symptoms.

The abovementioned receptor selectivity can be determined by the effect of the substances on cell lines which, after stable transfection with the corresponding cDNA, express the receptor subtypes in question [see the publication M. E. Olah, H. Ren, J. Ostrowski, K. A. Jacobson, G. L. Stiles, “Cloning, expression, and characterization of the unique bovine A1 adenosine receptor. Studies on the ligand binding site by site-directed mutagenesis”, J. Biol. Chem. 267 (1992), pages 10764-10770, the disclosure of which is hereby fully incorporated by way of reference].

The effect of the substances on such cell lines can be monitored by biochemical measurement of the intracellular messenger cAMP [see the publication K. N. Klotz, J. Hessling, J. Hegler, C. Owman, B. Kull, B. B. Fredholm, M. J. Lohse, “Comparative pharmacology of human adenosine receptor subtypes—characterization of stably transfected receptors in CHO cells”, Naunyn Schmiedebergs Arch. Pharmacol. 357 (1998), pages 1-9, the disclosure of which is hereby fully incorporated by way of reference].

The “adenosine-receptor-specific” ligands known from the literature are mainly derivatives based on natural adenosine [S.-A. Poulsen and R. J. Quinn, “Adenosine receptors: New opportunities for future drugs”, Bioorganic and Medicinal Chemistry 6 (1998), pages 619-641]. However, most adenosine ligands with this type of structure have the disadvantage that their action is not really receptor-specific, that their activity is less than that of natural adenosine or that they have only very weak activity after oral administration. Thus, they are mainly used only for experimental purposes. WO 01/25210, WO 02/070484, WO 02/070485 and WO 02/079195 describe 2-thio- and 2-oxy-3,5-dicyano-4-phenyl-6-aminopyridines substituted in various ways as adenosine receptor ligands for the treatment of disorders. WO 03/053441 describes specifically substituted 2-thio-3,5-dicyano-4-phenyl-6-aminopyridines as selective ligands of the adenosine A1 receptor, and WO 2006/027142 claims substituted phenylaminothiazole derivatives as dual adenosine A1/A2b agonists for the treatment of hypertension and other cardiovascular disorders. However, it was found that some of these compounds have disadvantages with respect to their physicochemical properties, such as, for example, their solubility and/or formulatability, or with respect to their in vivo properties, such as, for example, their pharmacokinetic behavior, their dose-activity relationship and/or their metabolization.

Furthermore, WO 01/62233 discloses various pyridine and pyrimidine derivatives and their use as adenosine receptor modulators. Substituted 3,5-dicyanopyridines as calcium-dependent potassium channel openers for treating urological disorders are claimed in EP 1 302 463-A1. The use of 2-amino-4-aryl-5-cyanopyridines as androgen receptor modulators is described in US 2005/0182105-A1. Furthermore, WO 98/06697 discloses compounds having a partial 4-phenoxypiperidine structure as muscarinic antagonists for the treatment of cognitive disorders.

It was an object of the present invention to provide novel compounds which act as selective agonists of the adenosine A1 and/or A2b receptor and which, as such, are suitable for the treatment and/or prevention in particular of cardiovascular disorders, such as hypertension metabolic syndrome, of diabetes and dyslipidemias and also for the protection of organs during transplantations and surgical interventions, and which additionally have an improved, angina pectoris, myocardial infarction, heart failure and atrial fibrillation, of profile compared to the property/compounds known from the prior art.

The present invention provides compounds of the formula (I)

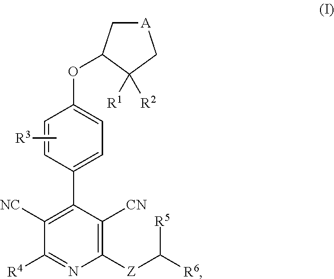

in which
A represents CH₂, CH₂CH₂, O, N—R⁷, S, S(═O) or S(═O)₂ in which
R⁷ represents hydrogen, (C₁-C₄)-alkyl, (C₁-C₄)-acyl or (C₁-C₄)-alkylsulfonyl, where the alkyl, acyl and alkylsulfonyl groups mentioned for their part may be substituted by hydroxyl, amino or carboxyl,
Z represents O or S,
R¹ represents hydrogen,
R² represents hydrogen, hydroxyl, amino, mono-(C₁-C₄)-alkylamino or di-(C₁-C₄)-alkylamino
or
R¹ and R² together with the carbon atom to which they are attached form a carbonyl group,
R³ represents hydrogen, halogen, cyano, (C₁-C₄)-alkyl or (C₁-C₄)-alkoxy, where the alkyl and alkoxy groups mentioned may be substituted up to three times by fluorine,
R⁴ represents a group of the formula —OR⁸ or —NR⁹R¹⁰ in which
R⁸ represents (C₁-C₆)-alkyl which may be mono- or disubstituted by identical or different substituents from the group consisting of hydroxyl, (C₁-C₄)-alkoxy, carboxyl and (C₁-C₄)-alkoxycarbonyl or may be substituted up to three times by fluorine, or represents (C₄-C₆)-cycloalkyl,
and
R⁹ and R¹⁹ are identical or different and independently of one another represent hydrogen or (C₁-C₆)-alkyl which may be substituted up to three times by fluorine or mono- or disubstituted by identical or different substituents from the group consisting of hydroxyl, (C₁-C₄)-alkoxy, amino, mono-(C₁-C₄)-alkylamino, di-(C₁-C₄)-alkylamino, carboxyl, (C₁-C₄)-alkoxycarbonyl and a 4- to 7-membered heterocycle,
where the heterocycle mentioned contains one or two ring heteroatoms from the group consisting of N, O and S and for its part may be mono- or disubstituted by identical or different substituents from the group consisting of (C₁-C₄)-alkyl, hydroxyl, oxo and (C₁-C₄)-alkoxy,
or
R⁹ and R¹⁰ together with the nitrogen atom to which they are attached form a 4- to 7-membered heterocycle which may contain a further ring heteroatom from the group consisting of N, O and S and may be mono- or disubstituted by identical or different substituents from the group consisting of fluorine, (C₁-C₄)-alkyl, hydroxyl, oxo, (C₁-C₄)-alkoxy, amino, mono-(C₁-C₄)-alkylamino, di-(C₁-C₄)-alkylamino, azetidino, pyrrolidino, piperidino and morpholino,
R⁵ represents hydrogen or (C₁-C₄)-alkyl
and
R⁶ represents (C₁-C₄)-alkyl which may be substituted by hydroxyl, (C₁-C₄)-alkoxy or up to three times by fluorine
or
represents (C₆-C₁₀)-aryl or 5- to 10-membered heteroaryl having up to three ring heteroatoms from the group consisting of N, O and S, each of which cycles may be
(i) mono- or disubstituted by identical or different radicals from the group consisting of halogen, nitro, cyano, (C₁-C₆)-alkyl, trifluoromethyl, hydroxyl, (C₁-C₆)-alkoxy, amino, mono-(C₁-C₆)-alkylamino, di-(C₁-C₆)-alkylamino, (C₁-C₆)-acylamino, (C₁-C₆)-alkylsulfonylamino, carboxyl, (C₁-C₆)-alkoxycarbonyl, aminocarbonyl, mono-(C₁-C₆)-alkylaminocarbonyl, di-(C₁-C₆)-alkylaminocarbonyl, aminosulfonyl, mono-(C₁-C₆)-alkylaminosulfonyl and di-(C₁-C₆)-alkylaminosulfonyl
and/or
(ii) substituted by pyrrolidino, piperidino, morpholino, piperazino, N′-(C₁-C₄)-alkylpiperazino, tetrazolyl or a group of the formula -L-R¹¹ in which
L represents a bond, NH or O
and
R¹¹ represents phenyl or 5- or 6-membered heteroaryl having up to three ring heteroatoms from the group consisting of N, O and S, each of which cycles may be mono- to trisubstituted by identical or different radicals from the group consisting of halogen, nitro, cyano, (C₁-C₆)-alkyl, trifluoromethyl, hydroxyl, (C₁-C₆)-alkoxy, difluoromethoxy, trifluoromethoxy, amino, mono-(C₁-C₆)-alkylamino, di-(C₁-C₆)-alkylamino, (C₁-C₆)-alkoxycarbonyl and carboxyl,
or N-oxides, salts, solvates, salts of the N-oxides or solvates of the N-oxides or salts thereof.

Compounds according to the invention are the compounds of the formula (I) and the salts, solvates and solvates of the salts thereof, the compounds which are encompassed by formula (I) and are mentioned in the formulae below, and the salts, solvates and solvates of the salts thereof, and the compounds which are encompassed by formula (I) and are mentioned below as exemplary embodiments, and the salts, solvates and solvates of the salts thereof, where the compounds which are encompassed by formula (I) and are mentioned below are not already salts, solvates and solvates of the salts.

The compounds according to the invention may, depending on their structure, exist in stereoisomeric forms (enantiomers, diastereomers). The invention therefore encompasses the enantiomers or diastereomers and respective mixtures thereof. The stereoisomerically pure constituents can be isolated from such mixtures of enantiomers and/or diastereomers in a known manner.

Where the compounds according to the invention can exist in tautomeric forms, the present invention encompasses all tautomeric forms.

Salts preferred for the purposes of the present invention are physiologically acceptable salts of the compounds according to the invention. Also included are salts which are not themselves suitable for pharmaceutical applications but can be used, for example, for the isolation or purification of the compounds according to the invention.

Physiologically acceptable salts of the compounds according to the invention include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases such as, by way of example and preferably, alkali metal salts (for example sodium and potassium salts), alkaline earth metal salts (for example calcium and magnesium salts) and ammonium salts derived from ammonia or organic amines having 1 to 16 carbon atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solvates refer for the purposes of the invention to those forms of the compounds according to the invention which form a complex in the solid or liquid state through coordination with solvent molecules. Hydrates are a specific form of solvates in which the coordination takes place with water. In the context of the present invention, preferred solvates are hydrates. In addition, the present invention also encompasses prodrugs of the compounds according to the invention. The term “prodrugs” encompasses compounds which for their part may be biologically active or inactive but are converted (for example metabolically or hydrolytically) into compounds according to the invention during their residence time in the body.

In the context of the present invention, the substituents have the following meaning, unless specified otherwise:

In the context of the invention, (C₁-C₆)-alkyl and (C₁-C₄)-alkyl represent a straight-chain or branched alkyl radical having 1 to 6 and 1 to 4 carbon atoms, respectively. Preference is given to a straight-chain or branched alkyl radical having 1 to 4 carbon atoms. The following radicals may be mentioned by way of example and by way of preference: methyl, ethyl, n-propyl, isopropyl, n-butyl, isobutyl, sec-butyl, tert-butyl, 1-ethylpropyl, n-pentyl and n-hexyl.

In the context of the invention, (C₄-C₆)-cycloalkyl represents a monocyclic saturated cycloalkyl group having 4 to 6 ring carbon atoms. The following radicals may be mentioned by way of example and by way of preference: cyclobutyl, cyclopentyl and cyclohexyl.

In the context of the invention, (C₁-C₆)-alkoxy and (C₁-C₄)-alkoxy represent a straight-chain or branched alkoxy radical having 1 to 6 and 1 to 4 carbon atoms, respectively. Preference is given to a straight-chain or branched alkoxy radical having 1 to 4 carbon atoms. The following radicals may be mentioned by way of example and by way of preference: methoxy, ethoxy, n-propoxy, isopropoxy, n-butoxy, tert-butoxy, n-pentoxy and n-hexoxy.

In the context of the invention, (C₁-C₆)-alkoxycarbonyl and (C₁-C₄)-alkoxycarbonyl represent a straight-chain or branched alkoxy radical having 1 to 6 and 1 to 4 carbon atoms, respectively, which is attached via a carbonyl group. Preference is given to a straight-chain or branched alkoxycarbonyl radical having 1 to 4 carbon atoms in the alkoxy group. The following radicals may be mentioned by way of example and by way of preference: methoxycarbonyl, ethoxycarbonyl, n-propoxycarbonyl, isopropoxycarbonyl, n-butoxycarbonyl and tert-butoxycarbonyl.

In the context of the invention, (C₁-C₆)-acyl and (C₁-C₄)-acyl [(C₁-C₆)-alkanoyl and (C₁-C₄)-alkanoyl] represent a straight-chain or branched alkyl radical having 1 to 6 and 1 to 4 carbon atoms, respectively, which carries a doubly attached oxygen atom in the 1-position and is attached via the 1-position. Preference is given to an acyl radical having 1 to 4 carbon atoms. The following radicals may be mentioned by way of example and by way of preference: formyl, acetyl, propionyl, n-butyryl, isobutyryl, n-pentanoyl, pivaloyl and n-hexanoyl.

In the context of the invention, (C₁-C₆)-acylamino represents an amino group having a straight-chain or branched acyl substituent which has 1 to 6 carbon atoms and is attached via the carbonyl group to the nitrogen atom. The following radicals may be mentioned by way of example and by way of preference: formylamino, acetylamino, propionylamino, n-butyrylamino, isobutyrylamino and pivaloylamino.

In the context of the invention, (C₁-C₆)-alkylsulfonyl and (C₁-C₄)-alkylsulfonyl represent a straight-chain or branched alkylsulfonyl radical having 1 to 6 and 1 to 4 carbon atoms, respectively. Preference is given to a straight-chain or branched alkylsulfonyl radical having 1 to 4 carbon atoms. The following radicals may be mentioned by way of example and by way of preference: methylsulfonyl, ethylsulfonyl, n-propylsulfonyl, isopropylsulfonyl, n-butylsulfonyl and tert-butylsulfonyl.

In the context of the invention, (C₁-C₆)-alkylsulfonylamino represents an amino group having a straight-chain or branched alkylsulfonyl substituent which has 1 to 6 carbon atoms and is attached via the sulfonyl group to the nitrogen atom. The following radicals may be mentioned by way of example and by way of preference: methylsulfonylamino, ethylsulfonylamino, n-propylsulfonylamino, isopropylsulfonylamino, n-butylsulfonylamino and tert-butylsulfonylamino.

In the context of the invention, mono-(C₁-C₆)-alkylamino and mono-(C₁-C₄)-alkylamino represent an amino group having a straight-chain or branched alkyl substituent which has 1 to 6 and 1 to 4 carbon atoms, respectively. Preference is given to a straight-chain or branched monoalkylamino radical having 1 to 4 carbon atoms. The following radicals may be mentioned by way of example and by way of preference: methylamino, ethylamino, n-propylamino, isopropylamino, n-butylamino, tert-butylamino, n-pentylamino and n-hexylamino.

In the context of the invention, di-(C₁-C₆)-alkylamino and di-(C₁-C₄)-alkylamino represent an amino group having two identical or different straight-chain or branched alkyl substituents having 1 to 6 and 1 to 4 carbon atoms, respectively. Preference is given to straight-chain or branched dialkylamino radicals having in each case 1 to 4 carbon atoms. The following radicals may be mentioned by way of example and by way of preference: N,N-dimethylamino, N,N-diethylamino, N-ethyl-N-methylamino, N-methyl-N-n-propylamino, N-isopropyl-N-n-propylamino, N,N-diisopropylamino, N-n-butyl-N-methylamino, N-tert-butyl-N-methylamino, N-ethyl-N-n-pentylamino and N-n-hexyl-N-methylamino.

In the context of the invention, mono- or di-(C₁-C₆)-alkylaminocarbonyl and mono- or di-(C₁-C₄)-alkylaminocarbonyl represent an amino group which is attached via a carbonyl group and which has one straight-chain or branched or two identical or different straight-chain or branched alkyl substituents each having 1 to 6 and 1 to 4 carbon atoms, respectively. Preference is given to a mono- or dialkylaminocarbonyl radical having in each case 1 to 4 carbon atoms in the alkyl group. The following radicals may be mentioned by way of example and by way of preference: methylaminocarbonyl, ethylaminocarbonyl, n-propylaminocarbonyl, isopropylaminocarbonyl, n-butylaminocarbonyl, tert-butylaminocarbonyl, N,N-dimethylaminocarbonyl, N,N-diethylaminocarbonyl, N-ethyl-N-methylaminocarbonyl, N-methyl-N-n-propylaminocarbonyl, N-n-butyl-N-methylaminocarbonyl and N-tert-butyl-N-methylaminocarbonyl.

In the context of the invention, mono- and di-(C₁-C₆)-alkylaminosulfonyl represent an amino group which is attached via a sulfonyl group and which has one straight-chain or branched or two identical or different straight-chain or branched alkyl substituents each having 1 to 6 carbon atoms. The following radicals may be mentioned by way of example and by way of preference: methylaminosulfonyl, ethylaminosulfonyl, n-propylaminosulfonyl, isopropylaminosulfonyl, n-butylaminosulfonyl, tert-butylaminosulfonyl, N,N-dimethylaminosulfonyl, N,N-diethylaminosulfonyl, N-ethyl-N-methylaminosulfonyl, N-methyl-N-n-propylaminosulfonyl, N-n-butyl-N-methylaminosulfonyl and N-tert-butyl-N-methylaminosulfonyl.

In the context of the invention, (C₆-C₁₀)-aryl represents an aromatic carbocycle having 6 or 10 ring carbon atoms. Preferred aryl radicals are phenyl and naphthyl.

In the context of the invention, a 4- to 7-membered heterocycle represents a saturated heterocycle having a total of 4 to 7 ring atoms which contains one or two ring heteroatoms from the group consisting of N, O and S and is attached via a ring carbon atom or, if appropriate, via a ring nitrogen atom. Preference is given to a 4- to 6-membered heterocycle having one or two ring heteroatoms from the group consisting of N and O. The following radicals may be mentioned by way of example: azetidinyl, oxetanyl, pyrrolidinyl, pyrazolidinyl, tetrahydrofuranyl, piperidinyl, piperazinyl, tetrahydropyranyl, morpholinyl, thiomorpholinyl, hexahydroazepinyl and hexahydro-1,4-diazepinyl. Azetidinyl, pyrrolidinyl, tetrahydrofuranyl, piperidinyl, piperazinyl, tetrahydropyranyl and morpholinyl are preferred.

In the context of the invention, 5- to 10-membered heteroaryl represents a mono- or optionally bicyclic aromatic heterocycle (heteroaromatic) which has a total of 5 to 10 ring atoms, contains up to three identical or different ring heteroatoms from the group consisting of N, O and S and is attached via a ring carbon atom or, if appropriate, via a ring nitrogen atom. The following radicals may be mentioned by way of example: furyl, pyrrolyl, thienyl, pyrazolyl, imidazolyl, thiazolyl, oxazolyl, isoxazolyl, isothiazolyl, triazolyl, oxadiazolyl, thiadiazolyl, pyridyl, pyrimidinyl, pyridazinyl, pyrazinyl, triazinyl, benzofuranyl, benzothienyl, benzimidazolyl, benzoxazolyl, benzothiazolyl, benzotriazolyl, indolyl, indazolyl, quinolinyl, isoquinolinyl, naphthyridinyl, quinazolinyl, quinoxalinyl, phthalazinyl, pyrazolo[3,4-b]pyridinyl. Monocyclic 5- or 6-membered heteroaryl radicals having up to two ring heteroatoms from the group consisting of N, O and S, such as, for example, furyl, thienyl, thiazolyl, oxazolyl, isothiazolyl, isoxazolyl, pyrazolyl, imidazolyl, pyridyl, pyrimidinyl, pyridazinyl, pyrazinyl, are preferred.

In the context of the invention, halogen includes fluorine, chlorine, bromine and iodine. Preference is given to chlorine or fluorine.

In the context of the invention, an oxo group represents an oxygen atom which is attached via a double bond to a carbon atom.

When radicals in the compounds according to the invention are substituted, the radicals may be mono- or polysubstituted, unless specified otherwise. For the purposes of the present invention, the meanings of all radicals which occur more than once are independent of one another. Preference is given to substitution by one, two or three identical or different substituents. Very particularly preferred is substitution by one or two identical or different substituents.

In the context of the present invention, preference is given to compounds of the formula (I) in which

A represents CH₂, CH₂CH₂, O or NH,
Z represents O or S,
R¹ represents hydrogen,
R² represents hydrogen, hydroxyl or amino,
R³ represents hydrogen, fluorine, chlorine, methyl or methoxy,
R⁴ represents a group of the formula —NR⁹R¹⁰ in which
R⁹ represents hydrogen,
R¹⁰ represents hydrogen or (C₁-C₄)-alkyl which may be mono- or disubstituted by identical or different substituents from the group consisting of hydroxyl, (C₁-C₄)-alkoxy, amino, mono-(C₁-C₄)-alkylamino and di-(C₁-C₄)-alkylamino
or
R⁹ and R¹⁰ together with the nitrogen atom to which they are attached form a 4- to 6-membered heterocycle which may contain a further ring heteroatom from the group consisting of N and O and may be mono- or disubstituted by identical or different substituents from the group consisting of (C₁-C₄)-alkyl, hydroxyl, (C₁-C₄)-alkoxy, amino, mono-(C₁-C₄)-alkylamino and di-(C₁-C₄)-alkylamino,
R⁵ represents hydrogen or methyl
and
R⁶ represents phenyl or 5- or 6-membered heteroaryl having up to two ring heteroatoms from the group consisting of N, O and S, each of which cycles may be
(i) mono- or disubstituted by identical or different radicals from the group consisting of fluorine, chlorine, cyano, (C₁-C₄)-alkyl, trifluoromethyl, (C₁-C₄)-alkoxy, amino, carboxyl, (C₁-C₄)-alkoxycarbonyl, aminocarbonyl and mono-(C₁-C₄)-alkylaminocarbonyl
and/or
(ii) substituted by a group of the formula -L-R¹¹ in which
L represents a bond or NH
and
R¹¹ represents phenyl or pyridyl, each of which may be mono- or disubstituted by identical or different radicals from the group consisting of fluorine, chlorine, cyano, (C₁-C₄)-alkyl, trifluoromethyl, (C₁-C₄)-alkoxy, trifluoromethoxy, (C₁-C₄)-alkoxycarbonyl and carboxyl,
or N-oxides, salts, solvates, salts of the N-oxides or solvates of the N-oxides or salts thereof.

In the context of the present invention, particular preference is given to compounds of the formula (I) in which

A represents CH₂ or O,
Z represents S,
R¹ represents hydrogen,
R² represents hydrogen or hydroxyl,
R³ represents hydrogen or fluorine,
R⁴ represents a group of the formula —NR⁹R¹⁰ in which
R⁹ represents hydrogen,
R¹⁰ represents hydrogen or (C₁-C₄)-alkyl which may be mono- or disubstituted by hydroxyl
or
R⁹ and R¹⁰ together with the nitrogen atom to which they are attached form an azetidino, pyrrolidino or piperidino ring, each of which may be substituted by hydroxyl,
R⁵ represents hydrogen
and
R⁶ represents phenyl, pyridyl, oxazolyl or thiazolyl, each of which may be
(i) mono- or disubstituted by identical or different radicals from the group consisting of fluorine, chlorine, cyano, methyl, trifluoromethyl, amino, carboxyl, methoxycarbonyl, ethoxycarbonyl, aminocarbonyl and methylaminocarbonyl
and/or
(ii) substituted by a group of the formula -L-R¹¹ in which
L represents a bond or NH
and
R¹¹ represents phenyl which may be mono- or disubstituted by identical or different radicals from the group consisting of fluorine, chlorine, methyl, trifluoromethyl, methoxycarbonyl, ethoxycarbonyl and carboxyl,
or N-oxides, salts, solvates, salts of the N-oxides or solvates of the N-oxides or salts thereof.

The individual definitions of radicals given in the respective combinations and preferred combinations of radicals are, independently of the respective given combination of radicals in question, also replaced by any radical definitions of other combinations.

Particular preference is given to combinations of two or more of the preferred ranges mentioned above.

The present invention furthermore provides a process for preparing the compounds of the formula (I) according to the invention in which R⁴ represents NH₂, characterized in that

[A] a compound of the formula (II)

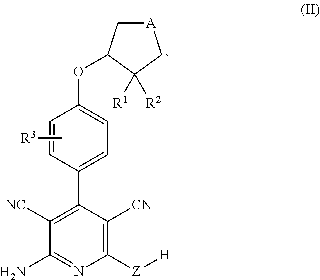

in which A, R¹, R², R³ and Z each have the meanings given above,
is reacted in an inert solvent in the presence of a base with a compound of the formula (III)

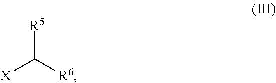

in which R⁵ and R⁶ have the meanings given above and
X represents a suitable leaving group, preferably halogen, in particular chlorine, bromine or iodine, or represents mesylate, tosylate or triflate,
or alternatively, if Z represents O,
[B] a compound of the formula (IV-A)

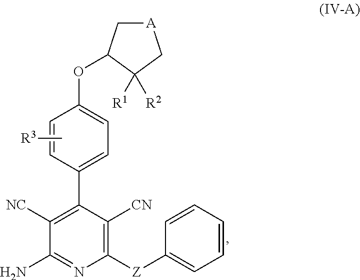

in which A, R¹, R² and R³ each have the meanings given above,
is reacted in an inert solvent in the presence of a base with a compound of the formula (V)

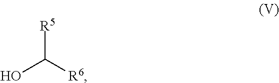

in which R⁵ and R⁶ have the meanings given above,
and the resulting compounds of the formula (I-A)

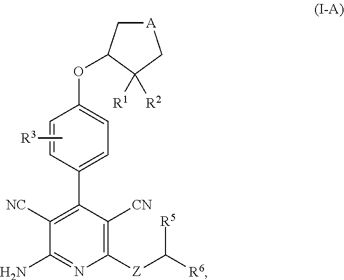

in which A, R¹, R², R³, R⁵, R⁶ and Z each have the meanings given above,
are, if appropriate, by methods known to the person skilled in the art, separated into their enantiomers and/or diastereomers and/or, if appropriate, converted with the appropriate (i) solvents and/or (ii) bases or acids into their solvates, salts and/or solvates of the salts.

The process described above can be illustrated in an exemplary manner by the reaction scheme below:

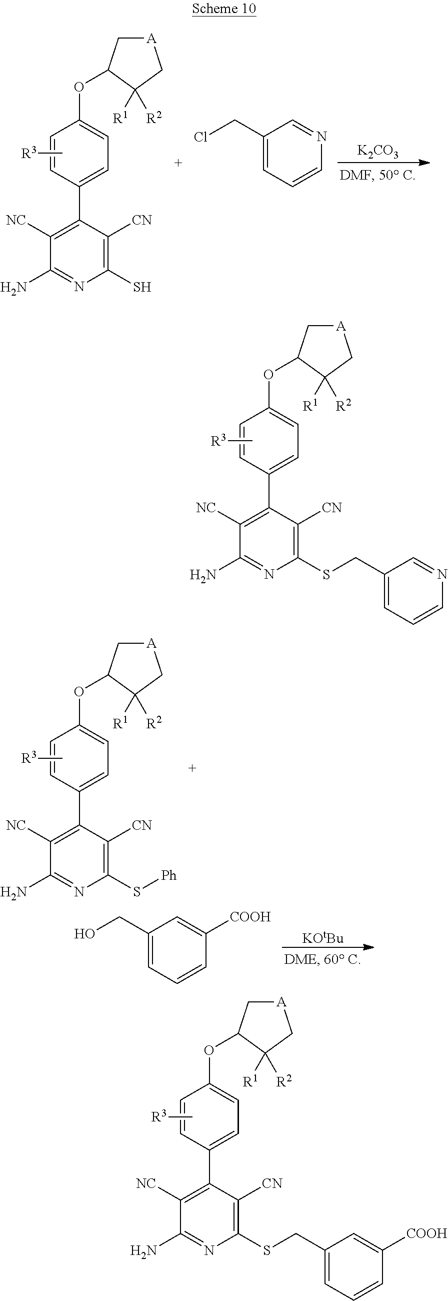

Suitable solvents for the reaction (II)+(III) are all organic solvents which are inert under the reaction conditions. These include ketones, such as acetone and methyl-ethyl ketone, acyclic and cyclic ethers, such as diethyl ether, methyl tert-butyl ether, 1,2-dimethoxyethane, tetrahydrofuran and dioxane, esters, such as ethyl acetate or butyl acetate, hydrocarbons, such as benzene, toluene, xylene, hexane and cyclohexane, chlorinated hydrocarbons, such as dichloromethane, trichloromethane and chlorobenzene, or other solvents, such as dimethylformamide (DMF), dimethyl sulfoxide (DMSO), N-methylpyrrolidinone (NMP), acetonitrile and pyridine. It is also possible to use mixtures of the solvents mentioned above. Preference is given to using dimethylformamide or N-methylpyrrolidinone.

Suitable bases for this reaction are the customary inorganic or organic bases. These preferably include alkali metal hydroxides, such as lithium hydroxide, sodium-hydroxide or potassium hydroxide, alkali metal carbonates, such as lithium carbonate, sodium carbonate, potassium carbonate or cesium carbonate, alkali metal bicarbonates, such as sodium bicarbonate or potassium bicarbonate, alkali metal alkoxides, such as sodium methoxide or potassium methoxide, sodium ethoxide or potassium ethoxide or potassium tert-butoxide, amides, such as sodium amide, lithium bis(trimethylsilyl)amide, sodium bis(trimethylsilyl)amide or potassium bis(trimethylsilyl)amide or lithium diisopropylamide, organometallic compounds, such as butyllithium or phenyllithium, or organic amines, such as triethylamine, diisopropylethylamine, pyridine, 1,8-diazabicyclo[5.4.0]undec-7-ene (DBU) or 1,5-diazabicyclo[4.3.0]non-5-ene (DBN). Preference is given to alkali metal carbonates and alkali metal bicarbonates, such as potassium carbonate and sodium bicarbonate.

Here, the base can be employed in an amount of from 1 to 10 mol, preferably from 1 to 5 mol, in particular from 1 to 3 mol, based on 1 mol of the compound of the formula (II).

The reaction (II)+(III) is generally carried out in a temperature range of from −78° C. to +150° C., preferably in the range from −20° C. to +120° C., in particular at from 0° C. to +80° C. (for Z=S) or from +20° C. to +100° C. (for Z=O). The reaction can be carried out at atmospheric, elevated or reduced pressure (for example in the range from 0.5 to 5 bar). The reaction is generally carried out at atmospheric pressure.

Suitable inert solvents for the reaction (IV-A)+(V) are in particular acyclic and cyclic ethers, such as diethyl ether, methyl tert-butyl ether, 1,2-dimethoxyethane, tetrahydrofuran and dioxane, hydrocarbons, such as benzene, toluene, xylene, hexane and cyclohexane, or dipolar solvents, such as dimethylformamide (DMF), dimethyl sulfoxide (DMSO), N-methylpyrrolidinone (NMP) and pyridine. It is also possible to use mixtures of these solvents. Preference is given to using 1,2-dimethoxyethane.

Suitable bases for this reaction are in particular alkali metal alkoxides, such as sodium methoxide or potassium methoxide, sodium ethoxide or potassium ethoxide or sodium tert-butoxide or potassium tert-butoxide, amides, such as sodium amide, lithium bis(trimethylsilyl)amide, sodium bis(trimethylsilyl)amide or potassium bis(trimethylsilyl)amide or lithium diisopropylamide, or organometallic compounds, such as butyllithium or phenyllithium. Preference is given to using potassium tert-butoxide.

Here, the base is generally employed in an amount of from 1 to 1.25 mol, preferably in an equimolar amount, based on 1 mol of the compound of the formula (V).

The reaction (IV-A)+(V) is generally carried out in a temperature range of from −20° C. to +120° C., preferably at from +20° C. to +100° C. The reaction can be carried out at atmospheric, elevated or reduced pressure (for example in the range from 0.5 to 5 bar). The reaction is generally carried out at atmospheric pressure.

The compounds of the formula (I) according to the invention in which R⁴ represents the group —NR⁹R¹⁰ where at least one of the two radicals R⁹ and R¹⁰ is not hydrogen can be prepared from the compounds of the formula (I-A) by initially converting these in a suitable solvent with isoamyl nitrite in the presence of copper(II) chloride or with sodium nitrite in the presence of hydrochloric acid into compounds of the formula (VI)

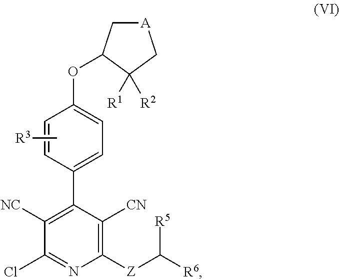

in which A, R¹, R², R³, R⁵, R⁶ and Z each have the meanings given above,
and then reacting the latter with a compound of the formula (VII)

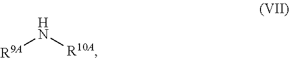

in which
R^{9A} has the meaning of R⁹ given above,
R^{10A} has the meaning of R¹⁰ given above,
but at least one of the two radicals R^{9A} and R^{10A} does not represent hydrogen,
to give compounds of the formula (I-B)

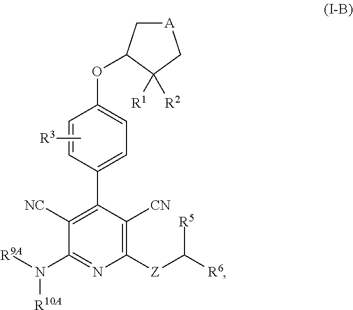

in which A, R¹, R², R³, R⁵, R⁶, R^{9A}, R^{10A} and Z each have the meanings given above and, if appropriate, separating the compounds of the formula (I-B) by methods known to the person skilled in the art into their enantiomers and/or diastereomers and/or, if appropriate, converting them with the appropriate (i) solvents and/or (ii) bases or acids into their solvates, salts and/or solvates of the salts.

The process described above can be illustrated by the reaction scheme below:

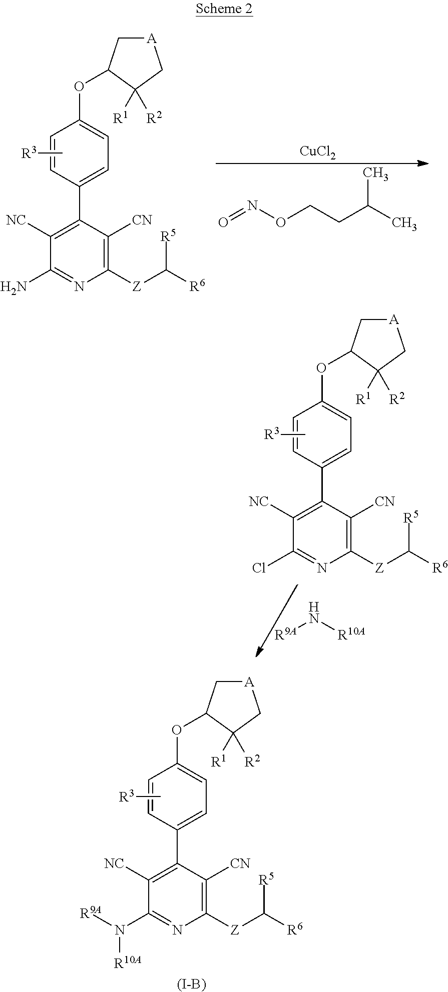

The reaction (I-A)→(VI) is generally carried out in a molar ratio of from 2 to 10 mol of isoamyl nitrite and 2 to 10 mol of copper(II) chloride per mole of the compound of the formula (I-A); alternatively, the reaction can also be carried out using 2 to 10 molar equivalents of sodium nitrite in hydrochloric acid.

Suitable solvents for the isoamyl nitrite/copper(II) chloride variant are all organic solvents which are inert under the reaction conditions. These include acyclic and cyclic ethers, such as diethyl ether and tetrahydrofuran, esters, such as ethyl acetate or butyl acetate, hydrocarbons, such as benzene, toluene, xylene, hexane and cyclohexane, chlorinated hydrocarbons, such as dichloromethane, 1,2-dichloroethane and chlorobenzene, or other solvents, such as dimethylformamide, acetonitrile or pyridine. It is also possible to use mixtures of the solvents mentioned above. Preferred solvents are acetonitrile and dimethylformamide.

If sodium nitrite is used, the solvent employed is preferably excess hydrochloric acid, if appropriate in combination with one of the organic solvents mentioned above.

The reaction is generally carried out in a temperature range of from −78° C. to +150° C., preferably in the range from −20° C. to +100° C., in particular at from +0° C. to +80° C. The reaction can be carried out at atmospheric, elevated or reduced pressure (for example in the range from 0.5 to 5 bar). The reaction is generally carried out at atmospheric pressure.

The reaction (VI)+(VII)→(I-B) is generally carried out in a molar ratio of from 1 to 8 mol of the compound of the formula (VII) per mole of the compound of the formula (VI).

Suitable solvents for the process step (VI)+(VII)→(I-B) are all organic solvents which are inert under the reaction conditions. These include alcohols, such as methanol, ethanol, n-propanol, isopropanol, n-butanol and tert-butanol, ketones, such as acetone and methyl ethyl ketone, acyclic and cyclic ethers, such as diethyl ether, 1,2-dimethoxyethane, tetrahydrofuran and dioxane, esters, such as ethyl acetate or butyl acetate, hydrocarbons, such as benzene, toluene, xylene, hexane and cyclohexane, chlorinated hydrocarbons, such as dichloromethane, 1,2-dichloroethane and chlorobenzene, or other solvents, such as dimethylformamide, acetonitrile, pyridine or dimethyl sulfoxide. Another suitable solvent is water. It is also possible to use mixtures of the solvents mentioned above. Preferred solvents are tetrahydrofuran and dimethylformamide.

The reaction is generally carried out in a temperature range of from 0° C. to +180° C., preferably in the range from +20° C. to +150° C., in particular at from +20° C. to +100° C. The reaction can be carried out at atmospheric, elevated or reduced pressure (for example in the range from 0.5 to 5 bar). The reaction is generally carried out at atmospheric pressure.

In an analogous manner, it is also possible to convert compounds of the formula (IV-A) into the corresponding substituted compounds of the formula (IV-B)

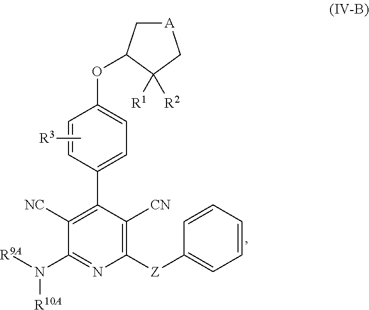

in which A, R¹, R², R³, R^{9A} and R^{10A} each have the meanings given above.

The compounds of the formula (II) in which Z is S can be prepared analogously to methods known from the literature by reacting aldehydes of the formula (VIII)

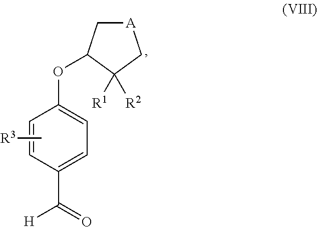

in which A, R¹, R² and R³ have the meanings given above,
in the presence of a base either with two equivalents of 2-cyanothioacetamide or initially with malononitrile and then with 2-cyanothioacetamide [see Scheme 3; cf., for example, Dyachenko et al., Russ. J. Chem. 33 (7), 1014-1017 (1997), 34 (4), 557-563 (1998); Dyachenko et al., Chemistry of Heterocyclic Compounds 34 (2), 188-194 (1998); Qintela et al., Eur. J. Med. Chem. 33, 887-897 (1998); Kandeel et al., Z. Naturforsch. 42b, 107-111 (1987); Reddy et al., J. Med. Chem. 49, 607-615 (2006); Evdokimov et al., Org. Lett. 8, 899-902 (2006)].

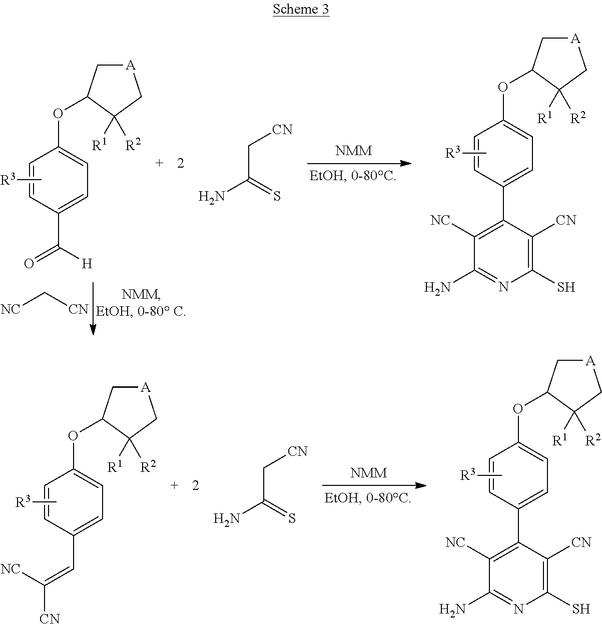

Alternatively, compounds of the formula (II) in which Z represents S can also be prepared from compounds of the formula (IV-A) by reaction with an alkali metal sulfide. This preparation method is illustrated by the formula scheme below:

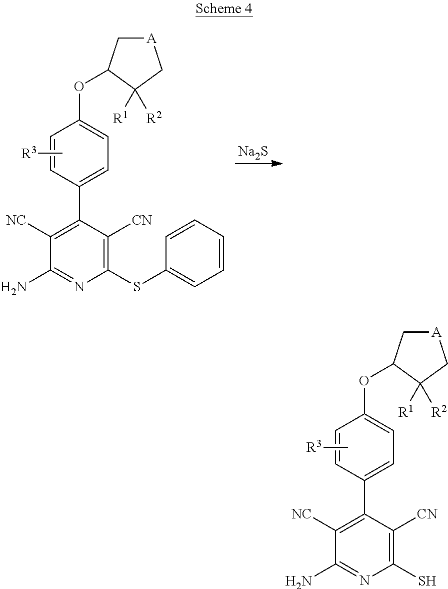

The alkali metal sulfide used is preferably sodium sulfide in an amount of from 1 to 10 mol, preferably from 1 to 8 mol, in particular from 1 to 5 mol, per mole of the compound of the formula (IV-A).

Suitable solvents for this process step are all organic solvents which are inert under the reaction conditions. These include alcohols, such as methanol, ethanol, n-propanol, isopropanol, n-butanol and tert-butanol, ketones, such as acetone and methyl ethyl ketone, acyclic and cyclic ethers, such as diethyl ether, 1,2-dimethoxyethane, tetrahydrofuran and dioxane, esters, such as ethyl acetate or butyl acetate, hydrocarbons, such as benzene, toluene, xylene, hexane and cyclohexane, chlorinated hydrocarbons, such as dichloromethane, 1,2-dichloroethane and chlorobenzene, or dipolar solvents, such as acetonitrile, pyridine, dimethylformamide, dimethyl sulfoxide or N-methylpyrrolidinone. Another suitable solvent is water. It is also possible to use mixtures of the solvents mentioned above. The preferred solvent is dimethylformamide.

The reaction is generally carried out in a temperature range of from 0° C. to +180° C., preferably in the range from +20° C. to +120° C., in particular at from +40° C. to +100° C. The reaction can be carried out at atmospheric, elevated or reduced pressure (for example in the range from 0.5 to 5 bar). The reaction is generally carried out at atmospheric pressure.

In an analogous manner, starting with compounds of the formula (IV-B), it is possible to obtain the corresponding N-substituted compounds of the formula (IX)

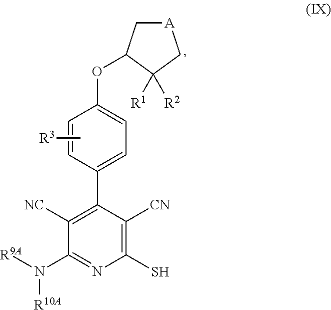

in which A, R¹, R², R³, R^{9A} and R^{10A} each have the meanings given above.

Compounds of the formula (II) in which Z represents O and N-substituted derivatives thereof can be obtained from compounds of the formula (IV-A) or (IV-B) by heating with an alkali metal hydroxide. This preparation method is illustrated by the reaction scheme below:

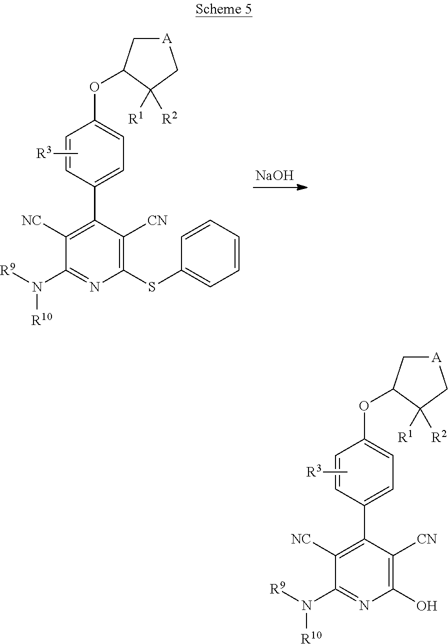

The alkali metal hydroxide used is preferably excess sodium hydroxide or potassium hydroxide. Suitable solvents are in particular alcohols, such as methanol, ethanol, n-propanol, isopropanol, n-butanol and tert-butanol and mixtures thereof with water. The reaction is generally carried out in a temperature range of from +20° C. to +120° C., preferably at from +50° C. to +100° C.

For their part, the compounds of the formula (IV-A) can be prepared from compounds of the formula (VIII) analogously to processes described in the literature [cf., for example, Kambe et al., Synthesis, 531-533 (1981); Elnagdi et al., Z. Naturforsch. 47b, 572-578 (1991); Reddy et al., J. Med. Chem. 49, 607-615 (2006); Evdokimov et al., Org. Lett. 8, 899-902 (2006)].

The compounds of the formula (I) according to the invention in which R⁴ represents the group —OR⁸ are obtainable, for example, from compounds of the formula (VI) analogously to the reaction (VI)+(VII)→(I-B) [see Scheme 6; cf., for example, D. Mabire et al., J. Med. Chem. 48, 2134-2153 (2005)]:

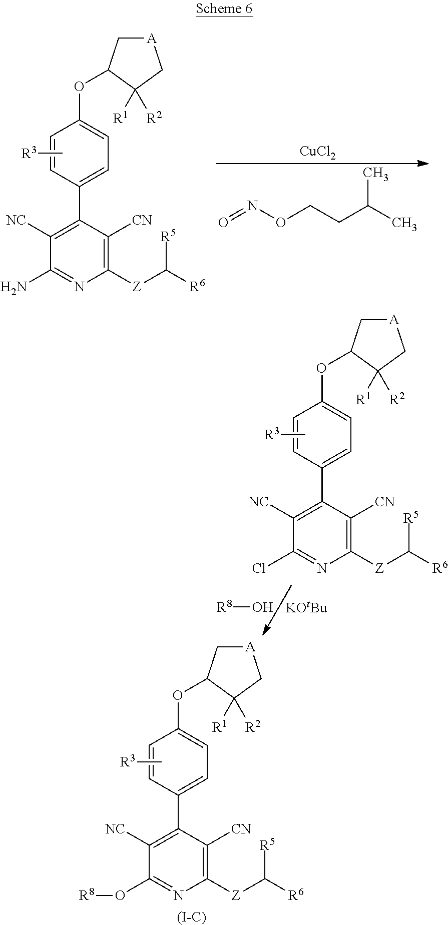

Such compounds of the formula (I-C) in which Z represents S can also be prepared analogously to the reaction sequences described above from compounds of the formula (VIII) by reaction with malononitrile and an appropriate alkoxide, subsequent N/S transformation and alkylation with a compound of the formula (III) (see Scheme 7; cf., for example, US 2005/0182105-A1):

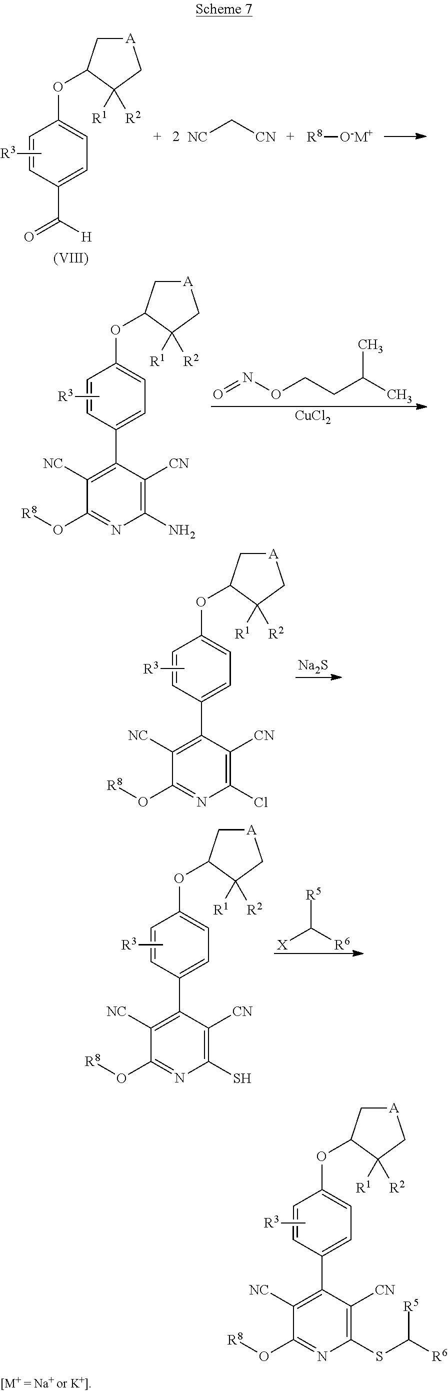

In a further alternative process, the compounds of the formula (I-C) can also be obtained by alkylation of compounds of the formula (X) (see Scheme 8):

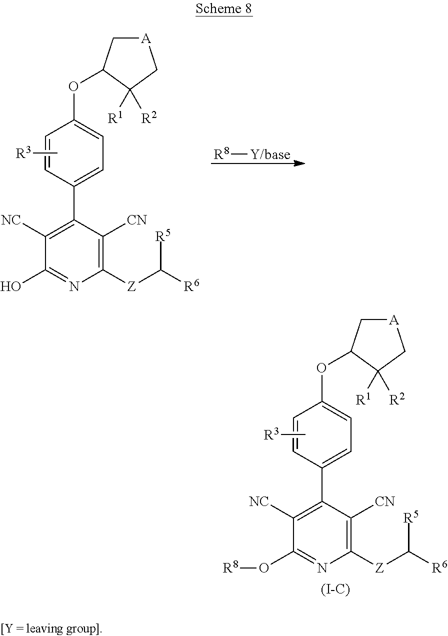

For their part, the compounds of the formula (X) are obtainable by methods known from the literature from compounds of the formula (VI) or (I-A) [cf., for example, G. Lavecchia et al., Tetrahedron Lett. 45, 6633-6636 (2004)].

The compounds of the formula (VIII) can be prepared analogously to processes described in the literature, for example via (A) ring opening of epoxides or (B) phenol ether formation under Mitsunobu conditions, in each case from 4-hydroxybenzaldehydes of the formula (XI) [see Scheme 9; cf., for example, R. Seemayer et al., Recl. Trav. Chim. Pays-Bas 110, 171 (1991); S. R. Adams et al., J. Am. Chem. Soc. 110, 3212 (1988); S. Matsunaga et al., J. Am. Chem. Soc. 122, 2252 (2000); D. L. Hughes, Org. Prep. Proceed. Int. 28, 127 (1996)]:

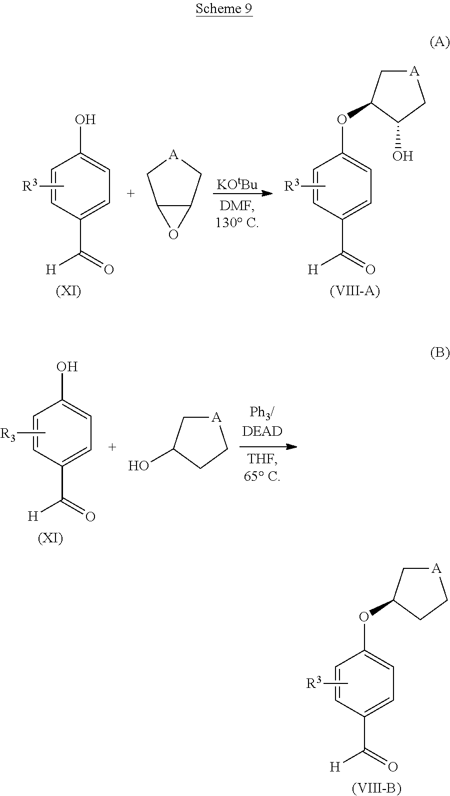

Compounds of the formula (VIII-A) obtained in this manner and having a trans-β-hydroxyl substituent in the phenolether head group can be converted by methods known from the literature into the corresponding cis-configured compounds (VIII-C) [see Scheme 10; cf., for example, M. Takahashi et al., Tetrahedron Asymmetry 6, 1617 (1995)]:

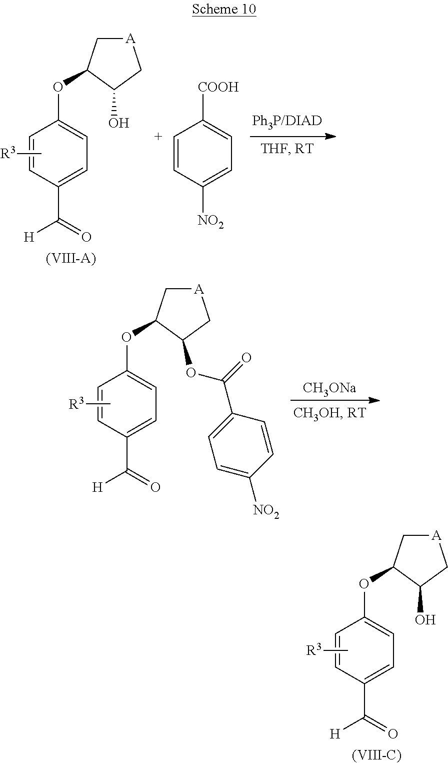

The compounds of the formula (III) are commercially available, known from the literature or can be prepared by methods known from the literature. Thus, by reaction of amides, thioamides or thiourea derivatives with a 1,3-dihaloacetone, it is possible to obtain, for example, substituted oxazole and thiazole derivatives of the formula (III-A), (III-B) and (III-C), respectively [see Scheme 11; cf., for example, I. Simiti et al., Chem. Ber. 95, 2672-2679 (1962); I. Simiti, E. Chindris, Arch. Pharm. (Weinheim) 304, 425-429 (1971)]:

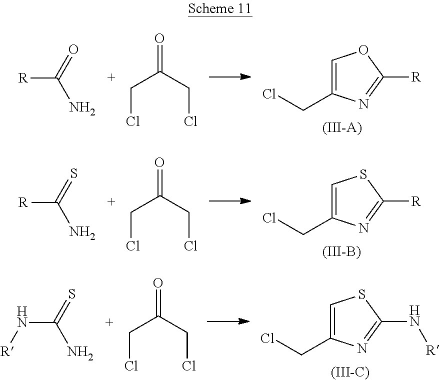

In the case of the compounds (III-C), these can be prepared and isolated either analogously to the literature, or they can be generated in situ and directly reacted further with a compound of the formula (II). The in situ generation with 1,3-dichloroacetone in dimethylformamide or ethanol as solvent is preferred.

2,5-Disubstituted oxazole derivatives according to formula (III) can be prepared analogously to processes known from the literature, for example as described in an exemplary manner in Reaction Scheme 12 below:

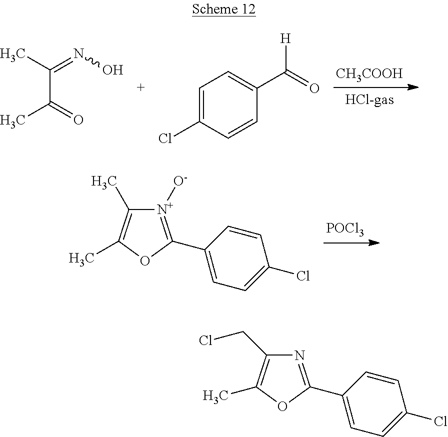

[cf., for example, Y. Goto et al., Chem. Pharm. Bull. 1971, 19, 2050-2057].

Oxazole derivatives according to formula (III) substituted in the 5-position can be obtained, for example, by reduction and subsequent halogenation of corresponding oxazole-4-carboxylic esters which for their part are obtainable from α-isocyanatoacetates by acylation (see Scheme 13):

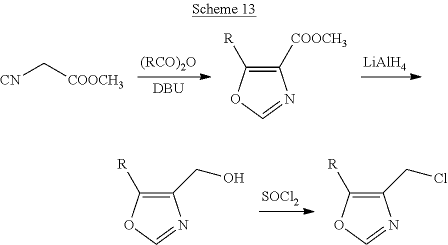

[cf., for example, M. Suzuki et al., J. Org. Chem. 1973, 38, 3571-3575].

2-Aryloxazole derivatives according to formula (III) can also be prepared via palladium-catalyzed coupling of arylboronic acids with 2-iodooxazole-4-carboxylic esters, as shown in an exemplary manner in Scheme 14:

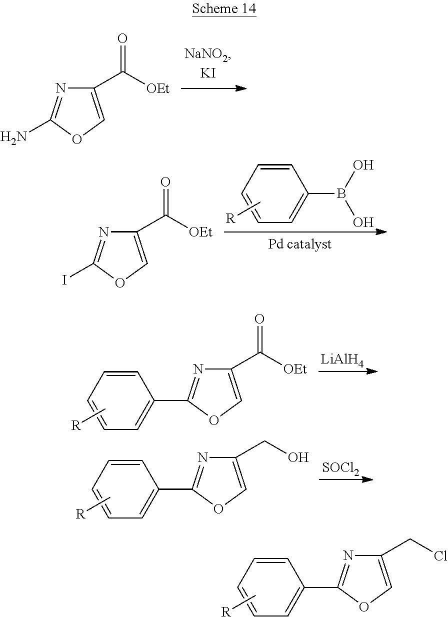

[cf., for example, E. A. Krasnokutskaya et al., Synthesis 2007, 1, 81-84; J. Hassan et al., Chem. Rev. 2002, 102, 1359-1469].

The compounds of the formula (V) are likewise commercially available or known from the literature, or they can be prepared analogously to processes described in the literature, for example similar to the compounds of the formula (III).

The compounds of the formulae (VII) and (XI) finally are either commercially available, are described as such in the literature or can be prepared by customary methods.

Further compounds according to the invention can, if appropriate, also be prepared by transforming functional groups of individual radicals and substituents, in particular those listed under R², R⁴, R⁶ and R⁷, starting with the compounds of the formula (I) obtained by the above processes. These transformations are carried out by customary methods known to the person skilled in the art and include, for example, reactions such as nucleophilic or electrophilic substitution, oxidation, reduction, hydrogenation, halogenation, alkylation, acylation, sulfonylation, amination, hydroxylation, the formation of carboxamides and carboxylic esters, ester cleavage and etherification.

Any functional groups which may be present in individual radicals and substituents—such as, in particular, amino, hydroxyl and carboxyl groups—may in the preparation processes described above, if expedient or required, also be present in temporarily protected form. The introduction and removal of such protective groups takes place in this connection by conventional methods known to the person skilled in the art [see, for example, T. W. Greene and P. G. M. Wuts, Protective Groups in Organic Synthesis, Wiley, New York, 1999; M. Bodanszky and A. Bodanszky, The Practice of Peptide Synthesis, Springer-Verlag, Berlin, 1984]. If a plurality of protective groups is present, the removal may optionally be carried out simultaneously in a one-pot reaction or in separate reaction steps.

Preferred amino protective groups are tert-butoxycarbonyl (Boc), benzyloxycarbonyl (Z) or p-tolylsulfonyl (tosyl). Suitable for protecting carboxyl groups are in particular the corresponding methyl, ethyl or tert-butyl esters. For a hydroxyl function, the protective group used is preferably benzyl or a silyl group, such as trimethylsilyl, tert-butyldimethylsilyl or dimethylphenylsilyl. If a 1,2- or 1,3-diol grouping is present, preference is given to using a ketal derived from symmetric ketones such as acetone or cyclohexanone (1,3-dioxolane or 1,3-dioxane) as common protective group.

Surprisingly, the compounds according to the invention have an unforeseeable useful pharmacological activity spectrum and are therefore particularly suitable for the prophylaxis and/or treatment of disorders, in particular cardiovascular disorders. Compared to the substances known from the prior art, the compounds according to the invention have an improved property profile, such as, in particular, increased solubility in physiological media and/or aqueous-organic solvent systems which are relevant for the formulation.

The pharmacological activity of the compounds according to the invention can be explained by their action as potent, selective ligands at adenosine A1 and/or A2b receptors. Here, they act as selective A1 agonists, as selective A2b agonists or as selective dual A1/A2b agonists.

In the context of the present invention, “selective ligands at adenosine A1 and/or A2b receptors” are adenosine receptor ligands where firstly a marked activity at A1 and/or A2b adenosine receptor subtypes and secondly no or a considerably weaker activity (by a factor of 10 or more) at A2a and A3 adenosine receptor subtypes can be observed, where with respect to the test methods for activity/selectivity, reference is made to the tests described in section B-1.

Depending on their respective structure, the compounds according to the invention can act as full or as partial adenosine receptor agonists. Partial adenosine receptor agonists are defined here as receptor ligands which trigger a functional response at adenosine receptors which is less than that of full agonists. Accordingly, partial agonists have lower activity with respect to receptor activation than full agonists.

The compounds of the formula (I), on their own or in combination with one or more other active compounds, are suitable for the prophylaxis and/or treatment of various disorders such as, for example, in particular hypertension and other disorders of the cardiovascular system (cardiovascular disorders), and cardioprotection following lesions of the heart, and of metabolic disorders.

In the context of the present invention, disorders of the cardiovascular system or cardiovascular disorders are to be understood as including, in addition to hypertension, for example in particular the following disorders: peripheral and cardial vascular disorders, coronary heart disease, coronary restenosis, such as, for example, restenosis after balloon dilation of peripheral blood vessels, acute coronary syndrome, stable and unstable angina pectoris, heart failure, tachycardias, arrhythmias, atrial and ventricular fibrillation and impaired peripheral circulation.

The compounds according to the invention are furthermore also particularly suitable for reducing the myocard region affected by an infarct, and also for the prophylaxis of secondary infarcts.

Furthermore, the compounds according to the invention are particularly suitable for the prophylaxis and/or treatment of thromboembolic disorders and ischemias, such as myocardial infarction, stroke and transitory ischemic attacks, and also for organ protection during transplantations and surgical interventions, for example on the heart.

Further indications for which the compounds according to the invention may be used are, for example, in particular the prophylaxis and/or treatment of disorders of the urogenital system, such as, for example, irritable bladder, erectile dysfunction and female sexual dysfunction, but in addition also the prophylaxis and/or treatment of inflammatory disorders, such as, for example, asthma and inflammatory dermatoses, of neuroinflammatory disorders of the central nervous system such as, for example, conditions following stroke, Alzheimer's disease and furthermore of neurodegenerative disorders, and also of pain, neoplastic diseases and nausea and emesis associated with cancer therapies.

A further indication is, for example, in particular the prophylaxis and/or treatment of disorders of the respiratory tract, such as, for example, asthma, chronic bronchitis, pulmonary emphysema, bronchiectasias, cystic fibrosis (mucoviscidosis) and pulmonary hypertension.

Finally, the compounds according to the invention are also suitable in particular for the prophylaxis and/or treatment of metabolic disorders, such as, for example, diabetes, in particular diabetes mellitus, diabetic sequelae, such as, for example, nephropathy and neuropathy, metabolic syndrome and also dyslipidemias.

The present invention furthermore provides the use of the compounds according to the invention for the treatment and/or prophylaxis of disorders, in particular the disorders mentioned above.

The present invention also provides the use of the compounds according to the invention for preparing a medicament for the treatment and/or prophylaxis of disorders, in particular the disorders mentioned above.

The present invention also provides a method for the treatment and/or prophylaxis of disorders, in particular the disorders mentioned above, using an effective amount of at least one compound according to the invention.

The compounds according to the invention can be used alone or, if required, in combination with other active compounds. The present invention furthermore provides medicaments comprising at least one compound according to the invention and one or more further active compounds, in particular for the treatment and/or prophylaxis of the disorders mentioned above.

Suitable active compounds for combinations are, by way of example and by way of preference: lipid metabolism-modifying active compounds, antidiabetics, hypotensive agents, perfusion-enhancing and/or antithrombotic drugs, antioxidants, chemokine receptor antagonists, p38-kinase inhibitors, NPY agonists, orexin agonists, anorectics, PAF-AH inhibitors, antiphlogistics (COX inhibitors, LTB₄-receptor antagonists) and analgesics such as, for example, aspirin.

The present invention provides in particular combinations comprising at least one of the compounds according to the invention and at least one lipid metabolism-modulating active compound, an antidiabetic, a hypotensive active compound and/or an antithrombotic agent.

Preferably, the compounds according to the invention can be combined with one or more lipid metabolism-modulating active compounds, by way of example and by way of preference from the group of the HMG-CoA reductase inhibitors, inhibitors of HMG-CoA reductase expression, squalene synthesis inhibitors, ACAT inhibitors, LDL receptor inductors, cholesterol absorption inhibitors, polymeric bile acid adsorbers, bile acid reabsorption inhibitors, MTP inhibitors, lipase inhibitors, LpL activators, fibrates, niacin, CETP inhibitors, PPAR-α, PPAR-γ and/or PPAR-δ agonists, RXR modulators, FXR modulators, LXR modulators, thyroid hormones and/or thyroid mimetics, ATP citrate lyase inhibitors, Lp(a) antagonists, cannabinoid receptor 1 antagonists, leptin receptor agonists, bombesin receptor agonists, histamine receptor agonists and the antioxidants/radical scavengers;

antidiabetics mentioned in the Rote Liste 2004/II, chapter 12, and also, by way of example and by way of preference, those from the group of the sulfonylureas, biguanides, meglitinide derivatives, glucosidase inhibitors, oxadiazolidinones, thiazolidinediones, GLP 1 receptor agonists, glucagon antagonists, insulin sensitizers, CCK 1 receptor agonists, leptin receptor agonists, inhibitors of liver enzymes involved in the stimulation of gluconeogenesis and/or glycogenolysis, modulators of glucose uptake and also potassium channel openers, such as, for example, those disclosed in WO 97/26265 and WO 99/03861;
hypotensive active compounds, by way of example and by way of preference from the group of the calcium antagonists, angiotensin All antagonists, ACE inhibitors, beta-receptor blockers, alpha-receptor blockers, diuretics, phosphodiesterase inhibitors, sGC stimulators, substances which increase the cGMP concentration, aldosterone antagonists, mineralocorticoid receptor antagonists, ECE inhibitors and the vasopeptidase inhibitors; and/or
antithrombotic agents, by way of example and by way of preference from the group of the platelet aggregation inhibitors or the anticoagulants.

Lipid metabolism-modulating active compounds are to be understood as meaning, preferably, compounds from the group of the HMG-CoA reductase inhibitors, squalene synthesis inhibitors, ACAT inhibitors, cholesterol absorption inhibitors, MTP inhibitors, lipase inhibitors, thyroid hormones and/or thyroid mimetics, niacin receptor agonists, CETP inhibitors, PPAR-α agonists, PPAR-γ agonists, PPAR-δ agonists, polymeric bile acid adsorbers, bile acid reabsorption inhibitors, antioxidants/radical scavengers and also the cannabinoid receptor 1 antagonists.

In a preferred embodiment of the invention, the compounds according to the invention are administered in combination with an HMG-CoA reductase inhibitor from the class of the statins, such as, by way of example and by way of preference, lovastatin, simvastatin, pravastatin, fluvastatin, atorvastatin, rosuvastatin, cerivastatin or pitavastatin.

In a preferred embodiment of the invention, the compounds according to the invention are administered in combination with a squalene synthesis inhibitor, such as, by way of example and by way of preference, BMS-188494 or TAK-475.

In a preferred embodiment of the invention, the compounds according to the invention are administered in combination with an ACAT inhibitor, such as, by way of example and by way of preference, avasimibe, melinamide, pactimibe, eflucimibe or SMP-797.

In a preferred embodiment of the invention, the compounds according to the invention are administered in combination with a cholesterol absorption inhibitor, such as, by way of example and by way of preference, ezetimibe, tiqueside or pamaqueside.

In a preferred embodiment of the invention, the compounds according to the invention are administered in combination with an MTP inhibitor, such as, by way of example and by way of preference, implitapide, BMS-201038, R-103757 or JTT-130.

In a preferred embodiment of the invention, the compounds according to the invention are administered in combination with a lipase inhibitor, such as, by way of example and by way of preference, orlistat.

In a preferred embodiment of the invention, the compounds according to the invention are administered in combination with a thyroid hormone and/or thyroid mimetic, such as, by way of example and by way of preference, D-thyroxine or 3,5,3′-triiodothyronine (T3).

In a preferred embodiment of the invention, the compounds according to the invention are administered in combination with an agonist of the niacin receptor, such as, by way of example and by way of preference, niacin, acipimox, acifran or radecol.

In a preferred embodiment of the invention, the compounds according to the invention are administered in combination with a CETP inhibitor, such as, by way of example and by way of preference, torcetrapib, JTT-705, BAY 60-5521, BAY 78-7499 or CETP vaccine (Avant).

In a preferred embodiment of the invention, the compounds according to the invention are administered in combination with a PPAR-γ agonist, such as, by way of example and by way of preference, pioglitazone or rosiglitazone.

In a preferred embodiment of the invention, the compounds according to the invention are administered in combination with a PPAR-δ agonist, such as, by way of example and by way of preference, GW-501516 or BAY 68-5042.

In a preferred embodiment of the invention, the compounds according to the invention are administered in combination with a polymeric bile acid adsorber, such as, by way of example and by way of preference, cholestyramine, colestipol, colesolvam, CholestaGel or colestimide.

In a preferred embodiment of the invention, the compounds according to the invention are administered in combination with a bile acid reabsorption inhibitor, such as, by way of example and by way of preference, ASBT (=IBAT) inhibitors, such as, for example, AZD-7806, S-8921, AK-105, BARI-1741, SC-435 or SC-635.

In a preferred embodiment of the invention, the compounds according to the invention are administered in combination with an antioxidant/radical scavenger, such as, by way of example and by way of preference, probucol, AGI-1067, BO-653 or AEOL-10150.

In a preferred embodiment of the invention, the compounds according to the invention are administered in combination with a cannabinoid receptor 1 antagonist, such as, by way of example and by way of preference, rimonabant or SR-147778.

Antidiabetics are to be understood as meaning, preferably, insulin and insulin derivatives, and also orally effective hypoglycemic active compounds. Here, insulin and insulin derivatives include both insulins of animal, human or biotechnological origin and also mixtures thereof. The orally effective hypoglycemic active compounds preferably include sulfonylureas, biguanides, meglitinide derivatives, glucosidase inhibitors and PPAR-γ agonists.

In a preferred embodiment of the invention, the compounds according to the invention are administered in combination with insulin.

In a preferred embodiment of the invention, the compounds according to the invention are administered in combination with a sulfonylurea, such as, by way of example and by way of preference, tolbutamide, glibenclamide, glimepiride, glipizide or gliclazide.

In a preferred embodiment of the invention, the compounds according to the invention are administered in combination with a biguanide, such as, by way of example and by way of preference, metformin.

In a preferred embodiment of the invention, the compounds according to the invention are administered in combination with a meglitinide derivative, such as, by way of example and by way of preference, repaglinide or nateglinide.

In a preferred embodiment of the invention, the compounds according to the invention are administered in combination with a glucosidase inhibitor, such as, by way of example and by way of preference, miglitol or acarbose.

In a preferred embodiment of the invention, the compounds according to the invention are administered in combination with a PPAR-γ agonist, for example from the class of the thiazolidinediones, such as, by way of example and by way of preference, pioglitazone or rosiglitazone.

The hypotensive agents are preferably understood as meaning compounds from the group of the calcium antagonists, angiotensin AII antagonists, ACE inhibitors, beta-receptor blockers, alpha-receptor blockers and diuretics.

In a preferred embodiment of the invention, the compounds according to the invention are administered in combination with a calcium antagonist, such as, by way of example and by way of preference, nifedipine, amlodipine, verapamil or diltiazem.

In a preferred embodiment of the invention, the compounds according to the invention are administered in combination with an angiotensin AII antagonist, such as, by way of example and by way of preference, losartan, valsartan, candesartan, embusartan, olmesartan or telmisartan.

In a preferred embodiment of the invention, the compounds according to the invention are administered in combination with an ACE inhibitor, such as, by way of example and by way of preference, enalapril, captopril, lisinopril, ramipril, delapril, fosinopril, quinopril, perindopril or trandopril.

In a preferred embodiment of the invention, the compounds according to the invention are administered in combination with a beta-receptor blocker, such as, by way of example and by way of preference, propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds according to the invention are administered in combination with an alpha-receptor blocker, such as, by way of example and by way of preference, prazosin.

In a preferred embodiment of the invention, the compounds according to the invention are administered in combination with a diuretic, such as, by way of example and by way of preference, furosemide, bumetanide, torsemide, bendroflumethiazide, chlorothiazide, hydrochlorothiazide, hydroflumethiazide, methyclothiazide, polythiazide, trichloromethiazide, chlorothalidone, indapamide, metolazone, quinethazone, acetazolamide, dichlorophenamide, methazolamide, glycerol, isosorbide, mannitol, amiloride or triamteren.

In a preferred embodiment of the invention, the compounds according to the invention are administered in combination with antisympathotonics, such as reserpine, clonidine or alpha-methyldopa, or in combination with potassium channel agonists, such as minoxidil, diazoxide, dihydralazine or hydralazine, or with substances which release nitrogen oxide, such as glycerol nitrate or sodium nitroprusside.

Antithrombotics are to be understood as meaning, preferably, compounds from the group of the platelet aggregation inhibitors or the anticoagulants.

In a preferred embodiment of the invention, the compounds according to the invention are administered in combination with a platelet aggregation inhibitor, such as, by way of example and by way of preference, aspirin, clopidogrel, ticlopidine or dipyridamol.

In a preferred embodiment of the invention, the compounds according to the invention are administered in combination with a thrombin inhibitor, such as, by way of example and by way of preference, ximelagatran, melagatran, bivalirudin or clexane.

In a preferred embodiment of the invention, the compounds according to the invention are administered in combination with a GPIIb/IIIa antagonist, such as, by way of example and by way of preference, tirofiban or abciximab.

In a preferred embodiment of the invention, the compounds according to the invention are administered in combination with a factor Xa inhibitor, such as, by way of example and by way of preference, rivaroxaban (BAY 59-7939), DU-176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds according to the invention are administered in combination with heparin or a low molecular weight (LMW) heparin derivative.

In a preferred embodiment of the invention, the compounds according to the invention are administered in combination with a vitamin K antagonist, such as, by way of example and by way of preference, coumarin.

The present invention furthermore provides medicaments comprising at least one compound according to the invention, usually together with one or more inert nontoxic pharmaceutically suitable auxiliaries, and also their use for the purposes mentioned above.

The compounds according to the invention can act systemically and/or locally. For this purpose, they can be administered in a suitable manner, such as, for example, orally, parenterally, pulmonally, nasally, sublingually, lingually, buccally, rectally, dermally, transdermally, conjunctivally, otically or as an implant or stent.

For these administration routes, the compounds according to the invention can be administered in suitable administration forms.

Suitable for oral administration are administration forms which work in accordance with the prior art and release the compounds according to the invention rapidly and/or in modified form and which comprise the compounds according to the invention in crystalline and/or amorphicized and/or dissolved form, such as, for example, tablets (uncoated or coated tablets, for example with enteric coats or coats which dissolve in a delayed manner or are insoluble and which control the release of the compound according to the invention), films/wafers or tablets which dissolve rapidly in the oral cavity, films/lyophilizates, capsules (for example hard or soft gelatin capsules), sugar-coated tablets, granules, pellets, powders, emulsions, suspensions, aerosols or solutions.

Parenteral administration may take place by circumventing a bioabsorption step (for example intravenously, intraarterially, intracardially, intraspinally or intralumbarly), or with bioabsorption (for example intramuscularly, subcutaneously, intracutaneously, percutaneously or intraperitoneally). Administration forms suitable for parenteral administration are inter alia preparations for injection or infusion in the form of solutions, suspensions, emulsions, lyophilizates or sterile powders.

Suitable for other administration routes are, for example, medicaments suitable for inhalation (inter alia powder inhalers, nebulizers), nose drops, solutions or sprays, tablets to be administered lingually, sublingually or buccally, films/wafers or capsules, suppositories, preparations to be administered to ears or eyes, vaginal capsules, aqueous suspensions (lotions, shaking mixtures), lipophilic suspensions, ointments, creams, transdermal therapeutic systems (for example plasters), milk, pastes, foams, powders for pouring, implants or stents.

Preference is given to oral or parenteral administration, in particular to oral and intravenous administration.

The compounds according to the invention can be converted into the administration forms mentioned. This can be carried out in a manner known per se by mixing with inert non-toxic pharmaceutically suitable auxiliaries. These auxiliaries include inter alia carriers (for example microcrystalline cellulose, lactose, mannitol), solvents (for example liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (for example antioxidants, such as, for example, ascorbic acid), colorants (for example inorganic pigments, such as, for example, iron oxides), and flavor and/or odor corrigents.

In general, it has been found to be advantageous in the case of parenteral administration to administer amounts of about 0.001 to 1 mg/kg, preferably about 0.01 to 0.5 mg/kg of body weight to obtain effective results. In the case of oral administration, the dosage is from about 0.01 to 100 mg/kg, preferably from about 0.01 to 20 mg/kg and very particularly preferably from 0.1 to 10 mg/kg of body weight.

In spite of this, it may be necessary to deviate from the amounts mentioned, namely depending on body weight, administration route, individual response to the active compound, the type of preparation and the time or the interval at which administration takes place. Thus, in some cases it may be sufficient to administer less than the above-mentioned minimum amount, whereas in other cases the upper limit mentioned has to be exceeded. In the case of the administration of relatively large amounts, it may be expedient to divide these into a plurality of individual doses which are administered over the course of the day.

The working examples below illustrate the invention. The invention is not limited to the examples.

The percentages in the tests and examples below are, unless indicated otherwise, percentages by weight; parts are parts by weight. Solvent ratios, dilution ratios and concentrations of liquid/liquid solutions are in each case based on volume.

### A. EXAMPLES

### Abbreviations and Acronyms Used

Ex. Example
CI chemical ionization (in MS)
d day(s)
DBU 1,8-diazabicyclo[5.4.0]undec-7-ene
TLC thin-layer chromatography
DCI direct chemical ionization (in MS)
DEAD diethyl azodicarboxylate
DIAD diisopropyl azodicarboxylate
DME 1,2-dimethoxyethane

### DMF N,N-dimethylformamide

DMSO dimethyl sulfoxide
ee enantiomeric excess
EA ethyl acetate
EI electron impact ionization (in MS)
ent enantiomerically pure/enantiomer
ESI electrospray ionization (in MS)
Et ethyl
EtOH ethanol
m.p. melting point
GC gas chromatography
sat. saturated
h hour(s)
HOAc acetic acid
HPLC high-pressure, high-performance liquid chromatography
conc. concentrated
KO^{t}Bu potassium tert-butoxide
cryst. crystalline, crystallized
LC-MS liquid chromatography-coupled mass spectrometry
LDA lithium diisopropylamide
Lit. literature (reference)
sol. solution
min minute(s)
MS mass spectrometry

### NMM N-methylmorpholine

NMR nuclear magnetic resonance spectrometry
PBS phosphate-buffered sodium chloride solution
PEG polyethylene glycol
Ph phenyl
quant. quantitative (in yield)
rac racemic, racemate
RP-HPLC reversed-phase HPLC
RT room temperature
Rₜ retention time (in HPLC)
TFA trifluoroacetic acid
THF tetrahydrofuran
dil. dilute
aq. aqueous

### HPLC, LC-MS and GC-MS Methods:

### Method 1 (HPLC):

Instrument: Hewlett Packard Series 1050; column: Symmetry TM C18 3.9×150 mm; flow rate: 1.5 ml/min; mobile phase A: water, mobile phase B: acetonitrile; gradient: →0.6 min 10% B→3.8 min 100% B→5.0 min 100% B→5.5 min 10% B; stop time: 6.0 min; injection volume: 10 μl; diode array detector signal: 214 and 254 nm.

### Method 2 (LC-MS):

MS instrument type: Micromass ZQ; HPLC instrument type: Waters Alliance 2795; column: Merck Chromolith SpeedROD RP-18e 100 mm×4.6 mm; mobile phase A: water+500 μl of 50% strength formic acid/l, mobile phase B: acetonitrile+500 μl of 50% strength formic acid/l; gradient: 0.0 min 10% B→7.0 min 95% B→9.0 min 95% B; oven: 35° C.; flow rate: 0.0 min 1.0 ml/min→7.0 min 2.0 ml/min→9.0 min 2.0 ml/min; UV detection: 210 nm.

### Method 3 (LC-MS):

MS instrument type: Micromass ZQ; HPLC instrument type: HP 1100 series; UV DAD; column: Phenomenex Gemini 3μ 30 mm×3.00 mm; mobile phase A: 1 of water+0.5 ml of 50% strength formic acid, mobile phase B: 1 of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

### Method 4 (LC-MS):

Instrument: Micromass Quattro LCZ with HPLC Agilent series 1100; column: Phenomenex Onyx Monolithic C18, 100 mm×3 mm; mobile phase A: 1 of water+0.5 ml of 50% strength formic acid, mobile phase B: 1 of acetonitrile+0.5 ml of 50% strength formic acid; gradient: 0.0 min 90% A→2 min 65% A→4.5 min 5% A→6 min 5% A; flow rate: 2 ml/min; oven: 40° C.; UV detection: 208-400 nm.

### Method 5 (LC-MS):

MS instrument type: Waters ZQ; HPLC instrument type: Waters Alliance 2795; column: Merck Chromolith RP-18e, 100 mm×3 mm; mobile phase A: 1 of water+0.5 ml of 50% strength formic acid, mobile phase B: 1 of acetonitrile+0.5 ml of 50% strength formic acid; gradient: 0.0 min 90% A→2 min 65% A→4.5 min 5% A→6 min 5% A; flow rate: 2 ml/min; oven: 40° C.; UV detection: 210 nm.

### Method 6 (LC-MS):

Instrument: Micromass Quattro LCZ with HPLC Agilent series 1100; column: Phenomenex Synergi 2.5μ MAX-RP 100A Mercury 20 mm×4 mm; mobile phase A: 1 of water+0.5 ml of 50% strength formic acid, mobile phase B: 1 of acetonitrile+0.5 ml of 50% strength formic acid; gradient: 0.0 min 90% A→0.1 min 90% A→3.0 min 5% A→4.0 min 5% A→4.1 min 90% A; flow rate: 2 ml/min; oven: 50° C.; UV detection: 208-400 nm.

### Method 7 (LC-MS):

MS instrument type: Micromass ZQ; HPLC instrument type: Waters Alliance 2795; column: Phenomenex Synergi 2.5μ MAX-RP 100A Mercury 20 mm×4 mm; mobile phase A: 1 of water+0.5 ml of 50% strength formic acid, mobile phase B: 1 of acetonitrile+0.5 ml of 50% strength formic acid; gradient: 0.0 min 90% A→0.1 min 90% A→3.0 min 5% A→4.0 min 5% A→4.01 min 90% A; flow rate: 2 ml/min; oven: 50° C.; UV detection: 210 nm.

### Method 8 (LC-MS):

Instrument: Micromass Platform LCZ with HPLC Agilent series 1100; column: Thermo Hypersil GOLD 3μ 20 mm×4 mm; mobile phase A: 1 of water+0.5 ml of 50% strength formic acid, mobile phase B: 1 of acetonitrile+0.5 ml of 50% strength formic acid; gradient: 0.0 min 100% A→0.2 min 100% A→2.9 min 30% A→3.1 min 10% A→5.5 min 10% A; flow rate: 0.8 ml/min; oven: 50° C.; UV detection: 210 nm.

### Method 9 (LC-MS):

Instrument: Micromass Quattro LCZ with HPLC Agilent series 1100; column: Phenomenex Synergi 2μ Hydro-RP Mercury 20 mm×4 mm; mobile phase A: 1 of water+0.5 ml of 50% strength formic acid, mobile phase B: 1 of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 208-400 nm.

### Method 10 (LC-MS):

MS instrument type: Micromass ZQ; HPLC instrument type: HP 1100 series; UV DAD; column: Phenomenex Synergi 2μ Hydro-RP Mercury 20 mm×4 mm; mobile phase A: 1 of water+0.5 ml of 50% strength formic acid, mobile phase B: 1 of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

### Method 11 (LC-MS):

MS instrument type: Micromass ZQ; HPLC instrument type: HP 1100 series; UV DAD; column: Phenomenex Synergi 2.5μ MAX-RP 100A Mercury 20 mm×4 mm; mobile phase A: 1 of water+0.5 ml of 50% strength formic acid, mobile phase B: 1 l of acetonitrile+0.5 ml of 50% strength formic acid; gradient: 0.0 min 90% A→0.1 min 90% A→3.0 min 5% A→4.0 min 5% A→4.1 min 90% A; flow rate: 2 ml/min; oven: 50° C.; UV detection: 210 nm.

### Method 12 (LC-MS):

MS instrument type: Micromass ZQ; HPLC instrument type: Waters Alliance 2795; column: Merck Chromolith SpeedROD RP-18e 100 mm×4.6 mm; mobile phase A: water+500 μl of 50% strength formic acid/l; mobile phase B: acetonitrile+500 μl of 50% strength formic acid/l; gradient: 0.0 min 10% B→7.0 min 95% B→9.0 min 95% B; flow rate: 0.0 min 1.0 ml/min→7.0 min 2.0 ml/min→9.0 min 2.0 ml/min; oven: 35° C.; UV detection: 210 nm.

### Method 13 (LC-MS):

MS instrument type: M-40 DCI (NH₃); HPLC instrument type: HP 1100 with DAD detection; column: Kromasil 100 RP-18, 60 mm×2.1 mm, 3.5 μm; mobile phase A: 5 ml of HClO₄ (70% strength)/liter of water, mobile phase B: acetonitrile; gradient: 0 min 2% B→0.5 min 2% B→4.5 min 90% B→6.5 min 90% B→6.7 min 2% B→7.5 min 2% B; flow rate: 0.75 ml/min; column temperature: 30° C.; UV detection: 210 nm.

### Method 14 (LC-MS):

Instrument: Micromass Quattro Premier with Waters HPLC Acquity; column: Thermo Hypersil GOLD 1.9μ 50 mm×1 mm; mobile phase A: 1 of water+0.5 ml of 50% strength formic acid, mobile phase B: 1 of acetonitrile+0.5 ml of 50% strength formic acid; gradient: 0.0 min 90% A→0.1 min 90% A→1.5 min 10% A→2.2 min 10% A; flow rate: 0.33 ml/min; oven: 50° C.; UV detection: 210 nm.

### Method 15 (Preparative HPLC):

HPLC instrument type: Abimed/Gilson Pump 305/306; Manometric Module 806; UV Knauer Variable Wavelength Monitor; column: Gromsil C18, 10 nm, 250 mm×30 mm; mobile phase A: 1 of water+0.5 ml of 99% strength trifluoroacetic acid, mobile phase B: 1 of acetonitrile; gradient: 0.0 min 2% B→10 min 2% B→50 min 90% B; flow rate: 20 ml/min; volume: 628 ml of A and 372 ml of B.

### Method 16 (HPLC):

HPLC instrument type: Agilent 1100 with DAD detection; column: Merck Chromolith SpeedROD RP-18e, 50 mm×4.6 mm; mobile phase A: 0.05% H₃PO₄, mobile phase B: acetonitrile; gradient: 0 min 5% B→2.5 min 95% B→3.0 min 95% B; flow rate: 5 ml/min; column temperature: 40° C.; UV detection: 210 nm.

### Method 17 (Preparative HPLC):

column: Grom-Sil C18, 10 μm, 250 mm×30 mm; mobile phase A: water+0.1% formic acid, mobile phase B: acetonitrile; flow rate: 50 ml/min; program: 0-5 min 10% B, 5-38 min gradient to 95% B; UV detection: 210 nm.

### Method 18 (Preparative HPLC):

column: YMC GEL ODS-AQ S-5, 15 μm; mobile phase gradient: acetonitrile/water 10:90→95:5.

### Method 19 (HPLC):

Instrument: HP 1100 with DAD detection; column: Kromasil 100 RP-18, 60 mm×2.1 mm, 3.5 μm; mobile phase A: 5 ml of HClO₄ (70% strength)/liter of water, mobile phase B: acetonitrile; gradient: 0 min 2% B→0.5 min 2% B→4.5 min 90% B→9 min 90% B→9.2 min 2% B→10 min 2% B; flow rate: 0.75 ml/min; column temperature: 30° C.; UV detection: 210 nm.

### Method 20 (LC-MS):

MS instrument type: Waters ZQ; HPLC instrument type: Agilent 1100 series; UV DAD; column: Thermo Hypersil GOLD 3μ 20 mm×4 mm; mobile phase A: 1 of water+0.5 ml of 50% strength formic acid, mobile phase B: 1 of acetonitrile+0.5 ml of 50% strength formic acid; gradient: 0.0 min 100% A→3.0 min 10% A→4.0 min 10% A→4.1 min 100% A (flow rate 2.5 ml/min); flow rate: 2 ml/min; oven: 55° C.; UV detection: 210 nm.

### Method 21 (GC-MS):

Instrument: Micromass GCT, GC 6890; column: Restek RTX-35, 15 m×200 μm×0.33 μm; constant helium flow: 0.88 ml/min; oven: 70° C.; inlet: 250° C.; gradient: 70° C., 30° C./min→310° C. (maintained for 3 min).

### Starting Materials and Intermediates

### Example 1A

### rac-trans-4-[(2-Hydroxycyclopentyl)oxy]benzaldehyde

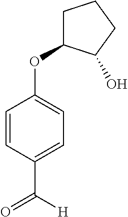

40 ml (38.84 g, 452.5 mmol) of cyclopentene oxide and 3.5 g (30.2 mmol) of potassium tert-butoxide were added to a solution of 36.85 g (301.7 mmol) of 4-hydroxybenzaldehyde in 100 ml of DMF, and the mixture was stirred at 130° C. for 4 h. A further 10 ml (9.71 g, 113.1 mmol) of cyclopentene oxide were then added, and stirring at 130° C. was continued for another 5 h. Most of the DMF was then distilled off under reduced pressure, and the residue was partitioned between ethyl acetate and water. The aqueous phase was reextracted three times with ethyl acetate, and the combined organic phases were washed with water and saturated sodium chloride solution, dried over sodium sulfate and concentrated. The oily crude product (66.4 g, 94% of theory; 88% pure according to LC-MS) was reacted further without purification. A sample for analysis was purified by preparative HPLC.

LC-MS (Method 3): Rₜ=1.81 min; MS (ESIpos): m/z=207 (M+H)⁺

¹H-NMR (400 MHz, DMSO-d₆): δ=9.87 (s, 1H); 7.86 (d, 2H); 7.13 (d, 2H); 5.08 (d, 1H); 4.62-4.56 (m, 1H); 4.11-4.04 (m, 1H); 2.23-2.12 (m, 1H); 1.93-1.83 (m, 1H); 1.83-1.50 (m, 4H).

### Example 2A

### rac-trans-4-[2-Hydroxycyclohexyl]oxy}benzaldehyde

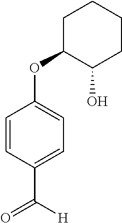

The title compound was prepared analogously to the procedure for Example 1A from 4-hydroxybenzaldehyde and cyclohexene oxide.

Yield: 100% of theory (94% pure according to LC-MS)

LC-MS (Method 3): Rₜ=1.8 min; MS (ESIpos): m/z=221 (M+H)⁺

¹H-NMR (500 MHz, DMSO-d₆): δ=9.95 (s, 1H); 7.82 (d, 2H); 7.15 (d, 2H); 4.98 (d, 1H); 4.22 (br., 1H); 3.55 (br., 1H); 2.05-1.98 (m, 1H); 1.91-1.83 (m, 1H); 1.67-1.48 (m, 2H); 1.40-1.20 (m, 4H).

### Example 3A

### rac-trans-4-{[4-Hydroxytetrahydrofuran-3-yl]oxy}benzaldehyde

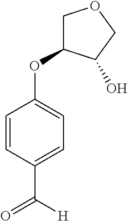

6.00 g (49.1 mmol) of 4-hydroxybenzaldehyde were initially charged in 16 ml of DMF, 6.34 g (73.7 mmol) of 3,4-epoxytetrahydrofuran and 551 mg (4.91 mmol) of potassium tert-butoxide were added and the mixture was stirred under argon at 120° C. for 20 h. The reaction mixture was then stirred into 200 ml of water, and the resulting suspension was extracted with 300 ml of ethyl acetate. The organic phase was washed with water and saturated aqueous sodium chloride solution, dried over magnesium sulfate, filtered and concentrated. The residue obtained was chromatographed on silica gel using the mobile phase dichloromethane/methanol (100:1).

Yield: 4.10 g (40% of theory)

LC-MS (Method 9): Rₜ=1.32 min; MS (ESIpos): m/z=209 [M+H]⁺

¹H-NMR (400 MHz, DMSO-d₆): δ=9.89 (s, 1H); 7.89 (d, 2H); 7.18 (d, 2H); 5.57 (d, 1H); 4.81 (d, 1H); 4.24 (br. t, 1H); 4.09 (dd, 1H); 3.93 (dd, 1H); 3.80 (d, 1H); 3.61 (dd, 1H).

### Example 4A

### rac-trans-4-{[4-{[tert-Butyl(dimethyl)silyl]oxy}tetrahydrofuran-3-yl]oxy}benzaldehyde

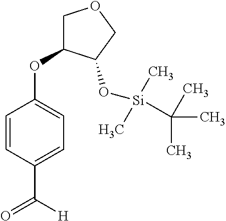

4.10 g (19.7 mmol) of the compound of Example 3A were initially charged in 80 ml of dichloromethane, 3.26 g (21.7 mmol) of tert-butyldimethylchlorosilane, 3.02 ml (21.7 mmol) of triethylamine and 96.2 mg (0.79 mmol) of 4-N,N-dimethylaminopyridine were added and the mixture was stirred at RT for 2 d. The solvent was then distilled off under reduced pressure, and the residue was suspended in 100 ml of cyclohexane. The precipitate was filtered off, the filtrate was concentrated and the residue was chromatographed on silica gel using the mobile phase cyclohexane/ethyl acetate (9:1).

Yield: 2.31 g (36% of theory)

LC-MS (Method 2): Rₜ=2.87 min; MS (ESIpos): m/z=323 [M+H]⁺

¹H-NMR (400 MHz, DMSO-d₆): δ=9.89 (s, 1H); 7.89 (d, 2H); 7.14 (d, 2H); 4.87 (d, 1H); 4.41 (br. t, 1H); 4.09 (dd, 1H); 4.01 (dd, 1H); 3.79 (d, 1H); 3.54 (dd, 1H); 0.88 (s, 9H); 0.09 (s, 3H); 0.07 (s, 3H).

### Example 5A

### rac-cis-2-(4-Formyl phenoxy)cyclopentyl 4-nitrobenzoate

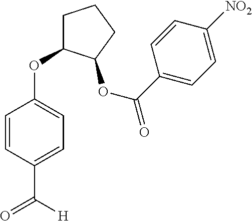

At about 10° C., 6.3 ml (30 mmol) of diisopropyl azodicarboxylate were added dropwise to a solution of 6.84 g (27.2 mmol) of the compound of Example 1A and 7.85 g (30 mmol) of triphenylphosphine in 50 ml of THF. After half of the addition, initially 1 g (6 mmol) of 4-nitrobenzoic acid was added, and after the end of the addition another 4 g (24 mmol) of 4-nitrobenzoic acid were added, and the mixture was stirred at RT overnight. Another 6.4 g (24 mmol) of triphenylphosphine and 5.2 ml (24.7 mmol) of diisopropyl azodicarboxylate were then added, and the mixture was stirred at RT for another night. The mixture was then concentrated under reduced pressure to give a yellow residue. This residue was chromatographed twice on silica gel using first toluene/ethyl acetate (10:1) and then isohexane/ethyl acetate (4:1) as mobile phases, which gave 1.33 g (14% of theory) of the title compound as a solid.

LC-MS (Method 3): Rₜ=2.71 min; MS (ESIpos): m/z=356 [M+H]⁺

¹H-NMR (400 MHz, DMSO-d₆): δ=9.8 (s, 1H); 8.28 (d, 2H); 8.02 (d, 2H); 7.78 (d, 2H); 7.11 (d, 2H); 5.50 (m, 1H); 5.11 (m, 1H); 2.25-2.11 (m, 2H); 1.97-1.83 (m, 3H); 1.76-1.62 (m, 1H).

### Example 6A

### rac-cis-4-{[-2-Hydroxycyclopentyl]oxy}benzaldehyde

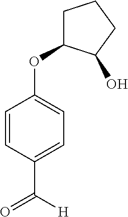

1.5 ml of a 30% strength solution of sodium methoxide in methanol were added to a solution of 1.33 g (3.74 mmol) of the compound of Example 5A in 33 ml of methanol, and the mixture was allowed to stand at RT overnight. The mixture was then concentrated, the residue was partitioned between ethyl acetate and water and the aqueous phase was extracted two more times with ethyl acetate. The combined organic phases were washed with saturated sodium chloride solution, dried over sodium sulfate and concentrated to give a dark oil (555 mg). Purification by preparative HPLC afforded 206 mg (27% of theory) of the title compound.

LC-MS (Method 3): Rₜ=1.85 min; MS (ESIpos): m/z=207 (M+H)⁺

¹H-NMR (400 MHz, DMSO-d₆): δ=9.86 (s, 1H); 7.84 (d, 2H); 7.13 (d, 2H); 4.71 (d, 1H); 4.71-4.62 (m, 1H); 4.19-4.10 (m, 1H); 2.08-1.94 (m, 1H); 1.88-1.60 (m, 4H); 1.60-1.46 (m, 4H).

### Example 7A

### rac-4-(Tetrahydrofuran-3-yloxy)benzaldehyde

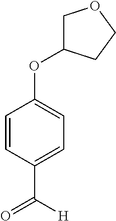

3.00 g (24.6 mmol) of 4-hydroxybenzaldehyde, 2.16 g (24.6 mmol) of 3-hydroxytetrahydrofuran and 9.67 g (36.8 mmol) of triphenylphosphine were dissolved in 100 ml of THF, and 16.0 g (36.8 mmol) of a 40% strength solution of diethyl azodicarboxylate in toluene was added a little at a time over a period of 15 min. The solution was heated under reflux for 4 h. Ethyl acetate was added after cooling, the mixture was washed with 0.5 N aqueous sodium hydroxide solution and saturated sodium chloride solution and the organic phase was dried over magnesium sulfate and concentrated. The residue was chromatographed on silica gel using the mobile phase cyclohexane/ethyl acetate (7:3).

Yield: 1.80 g (38% of theory)

LC-MS (Method 8): Rₜ=2.81 min; MS (ESIpos): m/z=193 (M+H)⁺

¹H-NMR (400 MHz, DMSO-d₆): δ=9.87 (s, 1H); 7.87 (d, 2H); 7.12 (d, 2H); 5.17 (t, 1H); 3.92 (dd, 1H); 3.88-3.74 (m, 3H); 2.29 (m, 1H); 1.99 (m, 1H).

### Example 8A

### rac-3-Methoxy-4-(tetrahydrofuran-3-yloxy)benzaldehyde

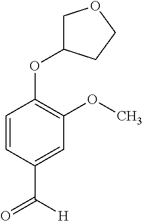

Analogously to the procedure for Example 7A, 10.0 g (65.7 mmol) of vanilin gave 5.57 g (38% of theory) of the title compound.

LC-MS (Method 3): Rₜ=1.53 min; MS (ESIpos): m/z=223 (M+H)⁺.

### Example 9A

### rac-3-Fluoro-4-(tetrahydrofuran-3-yloxy)benzaldehyde

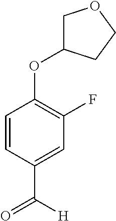

Analogously to the procedure for Example 7A, 5.15 g (36.7 mmol) of 3-fluoro-4-hydroxybenzaldehyde gave 1.61 g (21% of theory) of the title compound.

GC-MS (Method 21): Rₜ=5.95 min; MS (CIpos): m/z=211 (M+H)⁺.

### Example 10A

### rac-tert-Butyl 3-(4-formyl phenoxy)pyrrolidine-1-carboxylate

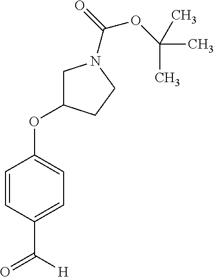

2.00 g (16.4 mmol) of 4-hydroxybenzaldehyde, 3.07 g (16.4 mmol) of tert-butyl 3-hydroxypyrrolidine-1-carboxylate and 6.44 g (24.6 mmol) of triphenylphosphine were dissolved in 67 ml of THF, and 10.7 g (24.6 mmol) of a 40% strength solution of diethyl azodicarboxylate in toluene were added a little at a time over a period of 15 min. The solution was heated under reflux for 2 h and then concentrated, and the residue was chromatographed on silica gel using the mobile phase cyclohexane/ethyl acetate (7:3).

Yield: 1.89 g (38% of theory)

LC-MS (Method 3): Rₜ=2.44 min; MS (ESIpos): m/z=292 (M+H)⁺

¹H-NMR (400 MHz, DMSO-d₆): δ=9.88 (s, 1H); 7.87 (d, 2H); 7.15 (d, 2H); 5.16 (br., 1H); 3.60 (m, 1H); 3.47-3.29 (m, 3H); 2.19 (m, 1H); 2.08 (m, 1H).

### Example 11A

### rac-trans-4-({4-Hydroxy-1-[(4-methylphenyl)sulfonyl]pyrrolidin-3-yl}oxy)benzaldehyde

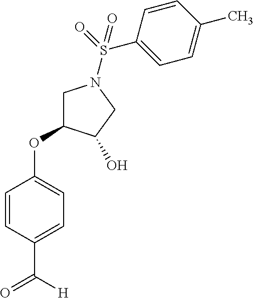

1.00 g (8.19 mmol) of 4-hydroxybenzaldehyde was initially charged in 2.7 ml of DMF, 2.74 g (11.5 mmol) of 3-[(4-methylphenyl)sulfonyl]-6-oxa-3-azabicyclo[3.1.0]hexane [D. M. Hodgson, T. J. Miles, J. Witherington, Tetrahedron 59 (49), 9729-9742 (2003)] and 91.9 mg (0.82 mmol) of potassium tert-butoxide were added and the mixture was stirred under argon at 130° C. for 7 h. The reaction mixture was then stirred into 50 ml of water and stirred at 50° C. for 1 h. The precipitate formed was filtered off, washed with water, dried and reacted without further purification.

Yield: 3.01 g (87% pure according to LC-MS, 88% of theory)

LC-MS (Method 3): Rₜ=1.32 min; MS (ESIpos): m/z=323 [M+H]⁺.

### Example 12A

### rac-trans-4-(2-Hydroxycyclopentyl)oxybenzylidenemalononitrile

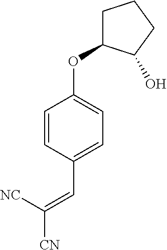

5.0 g (21.8 mmol) of the compound of Example 1A were dissolved in 45 ml of ethanol, 1.44 g (21.8 mmol) of malononitrile and 48.0 μl (0.436 mmol) of 4-methylmorpholine were added and the mixture was heated under reflux for 3 h. The solvent was then distilled off under reduced pressure. The residue solidified after scratching with a glass rod and was processed further without further purification.

Yield: 6.35 g (81% pure according to LC-MS, 93% of theory)

LC-MS (Method 10): Rₜ=2.37 min; MS (ESIpos): m/z=255 [M+H]⁺

¹H-NMR (400 MHz, DMSO-d₆): δ=8.41 (s, 1H); 7.98 (d, 2H); 7.20 (d, 2H); 5.12 (d, 1H); 4.63-4.59 (m, 1H); 4.08 (br., 1H); 2.22-2.12 (m, 1H); 1.93-1.82 (m, 1H); 1.82-1.50 (m, 1H).

### Example 13A

### rac-trans-4-(2-Hydroxycyclohexyl)oxybenzylidenemalononitrile

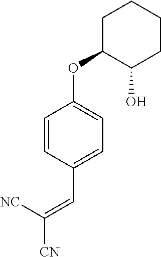

The title compound was prepared analogously to the procedure for Example 12A from the compound of Example 2A.

Yield: 92% of theory (crude product, 92% pure according to LC-MS)

LC-MS (Method 10): Rₜ=2.43 min; MS (ESIpos): m/z=269 [M+H]⁺.

### Example 14A

### rac-trans-(4-{[4-{[tert-Butyl(dimethyl)silyl]oxy}tetrahydrofuran-3-yl]oxy}benzylidene)-malononitrile

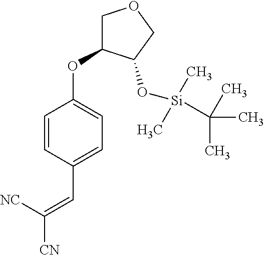

2.25 g (6.98 mmol) of the compound of Example 4A were dissolved in 20 ml of ethanol, 484 mg (7.33 mmol) of malononitrile and 15.0 μl (0.14 mmol) of 4-methylmorpholine were added and the mixture was heated under reflux for 3 h. The solvent was distilled off under reduced pressure and the residue was processed further directly, without further purification.

Yield: 2.49 g (96% of theory)

¹H-NMR (400 MHz, DMSO-d₆): δ=8.41 (s, 1H); 7.98 (d, 2H); 7.19 (d, 2H); 4.89 (d, 1H); 4.41 (br. t, 1H); 4.09 (dd, 1H); 4.01 (dd, 1H); 3.78 (d, 1H); 3.53 (dd, 1H); 0.88 (s, 9H); 0.08 (s, 3H); 0.07 (s, 3H).

### Example 15A

### rac-trans-(4-{[4-Hydroxytetrahydrofuran-3-yl]oxy}benzylidene)malononitrile

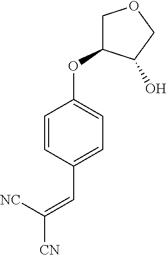

4.00 g (19.21 mmol) of the compound of Example 3A were dissolved in 60 ml of ethanol, 1.33 g (20.17 mmol) of malononitrile and 42 μl (0.384 mmol) of 4-methylmorpholine were added and the mixture was heated under reflux for 3 h. The reaction solution was then used directly, without work-up.

### Example 16A

### rac-cis-(4-{[-2-Hydroxycyclopentyl]oxy}benzylidene)malononitrile

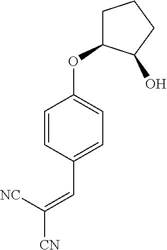

195 mg (0.364 mmol) of the compound of Example 6A were dissolved in 3 ml of ethanol, 12 mg (0.18 mmol) of malononitrile and 4 μl (0.036 mmol) of 4-methylmorpholine were added and the mixture was heated under reflux for 1 h. Two more times, another 12 mg (0.18 mmol) of malononitrile and 4 μl (0.036 mmol) of 4-methylmorpholine were then added, and in each case the mixture was heated under reflux for another 4 h. The mixture was then concentrated to dryness under reduced pressure, and the residue was purified by preparative HPLC.

Yield: 55 mg (74% pure according to LC-MS, 59% of theory)

LC-MS (Method 3): Rₜ=2.16 min; MS (ESIpos): m/z=255 [M+H]⁺.

### Example 17A

### rac-[4-(Tetrahydrofuran-3-yloxy)benzylidene]malononitrile

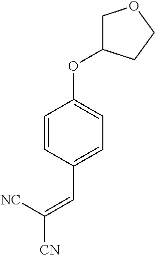

1.60 g (8.32 mmol) of the compound of Example 7A were dissolved in 24 ml of ethanol, 0.58 mg (8.74 mmol) of malononitrile and 92 μl (0.83 mmol) of 4-methylmorpholine were added and the mixture was heated under reflux for 2 h. The reaction solution was then used directly, without work-up.

### Example 18A

### tert-Butyl 3-[4-(2,2-dicyanovinyl)phenoxy]pyrrolidine-1-carboxylate

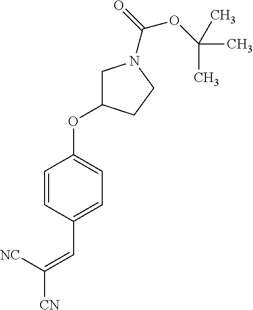

1.80 g (6.18 mmol) of the compound of Example 10A were dissolved in 18 ml of ethanol, 0.43 g (6.49 mmol) of malononitrile and 68 μl (0.62 mmol) of 4-methylmorpholine were added and the mixture was heated under reflux for 1 h. The reaction solution was then used directly, without work-up.

### Example 19A

### rac-trans-2-Amino-4-(4-{[-2-hydroxycyclopentyl]oxy}phenyl)-6-mercaptopyridine-3,5-dicarbonitrile

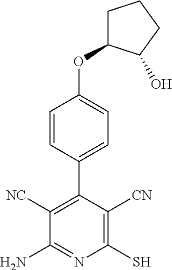

2.5 ml (23 mmol) of N-methylmorpholine were added to 2.9 g (11 mmol) of the compound of Example 12A and 0.65 g (5.5 mmol) of 2-cyanothioacetamide in 32 ml of ethanol, and the mixture was heated under reflux for 3 h. A further 0.53 g (4.5 mmol) of 2-cyanothioacetamide was then added, and the mixture was heated under reflux for another 1 h. 11.4 ml of 2 N hydrochloric acid were then added, and the mixture was stirred at RT for 1 h and concentrated under reduced pressure to give a brown oil (7 g). 6.5 g of this residue were chromatographed on silica gel using the mobile phases dichloromethane and dichloromethane/methanol (0.5% to 10%). This gave 1.33 g (34% of theory) after concentration of the product-containing fractions. Trituration with dichloromethane/methyl tert-butyl ether afforded 580 mg (14% of theory) of the pure title compound as a solid.

LC-MS (Method 2): Rₜ=1.60 min; MS (ESIpos): m/z=353 [M+H]⁺

¹H-NMR (500 MHz, DMSO-d₆): δ=13.10-12.85 (br., 1H); 8.20-7.60 (br., 2H); 7.46 (d, 2H); 7.10 (d, 2H); 5.05 (br., 1H); 4.57-4.51 (m, 1H); 4.12-4.06 (m, 1H); 2.21-2.11 (m, 1H); 1.93-1.82 (m, 1H); 1.81-1.60 (m, 3H); 1.60-1.51 (m, 1H).

### Example 20A

### rac-trans-2-Amino-4-(4-{[-2-hydroxycyclohexyl]oxy}phenyl)-6-mercaptopyridine-3,5-dicarbonitrile

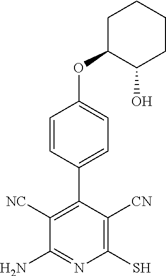

The title compound was obtained analogously to the procedure for Example 19A from the compound of Example 13A. Purification was by dissolution of the residue obtained after treatment with 2 N hydrochloric acid in dilute aqueous sodium hydroxide solution, extraction with ethyl acetate, acidification of the aqueous phase with dilute hydrochloric acid and another extraction with ethyl acetate. The second ethyl acetate phase was concentrated and the residue was dried under high vacuum. This crude product was used for the next step without further purification.

Yield: 69% of theory (79% pure according to LC-MS)

LC-MS (Method 9): Rₜ=2.01 min; MS (ESIpos): m/z=367 [M+H]⁺

¹H-NMR (400 MHz, DMSO-d₆): δ=13.05-12.85 (br. s, 1H); 8.20-7.60 (br. s, 2H); 7.43 (d, 2H); 7.12 (d, 2H); 4.95 (br. d, 1H); 4.15 (br. m, 1H); 3.60-3.45 (br., 1H); 2.05 (m, 1H); 1.90 (m, 1H); 1.63 (m, 2H); 1.44-1.20 (m, 4H).

### Example 21A

### rac-trans-2-Amino-4-(4-{[4-{[tert-butyl(dimethyl)silyl]oxy}tetrahydrofuran-3-yl]oxy}-phenyl)-6-mercaptopyridine-3,5-dicarbonitrile

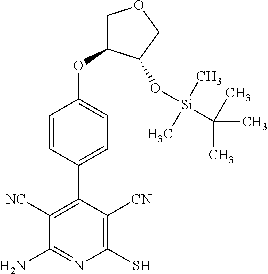

54.1 mg (0.54 mmol) of 2-cyanothioacetamide and 59.0 μl (0.54 mmol) of 4-methylmorpholine were added to a solution of 100 mg (0.27 mmol) of the compound of Example 14A in 3.0 ml of ethanol, and the mixture was heated under reflux for 18 h. 6 ml of 1 N hydrochloric acid were then added to the reaction mixture. The resulting precipitate was decanted off, and the residue was washed with water and purified by preparative HPLC (column: YMC GEL ODS-AQ S-5, 15 μm; mobile phase gradient: acetonitrile/water 10:90→95:5).

Yield: 20 mg (85% pure according to LC-MS, 13% of theory)

¹H-NMR (400 MHz, DMSO-d₆): δ=12.9 (br. s, 1H); 8.10-7.60 (br. s, 2H); 7.48 (d, 2H); 7.11 (d, 2H); 4.81 (d, 1H); 4.41 (br. t, 1H); 4.09 (dd, 1H); 4.01 (dd, 1H); 3.79 (d, 1H); 3.55 (dd, 1H); 0.88 (s, 9H); 0.09 (s, 3H); 0.07 (s, 3H).

LC-MS (Method 2): Rₜ=2.52 min; MS (ESIpos): m/z=469 [M+H]⁺.

### Example 22A

### rac-trans-2-Amino-4-(4-{[4-hydroxytetrahydrofuran-3-yl]oxy}phenyl)-6-mercaptopyridine-3,5-dicarbonitrile

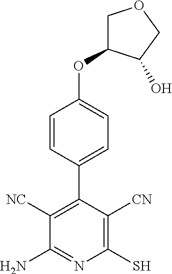

4.92 g (19.2 mmol) of the compound of Example 15A were dissolved in 60 ml of ethanol, 2.11 g (21.1 mmol) of 2-cyanothioacetamide and 2.32 ml (21.1 mmol) of 4-methylmorpholine were added and the mixture was heated under reflux for 18 h. The solvent was then distilled off under reduced pressure, and the residue was processed directly, without further purification.

Yield: 8.30 g (50% pure according to LC-MS, 64% of theory)

LC-MS (Method 9): Rₜ=1.45 min; MS (ESIpos): m/z=355 [M+H]⁺.

### Example 23A

### rac-cis-2-Amino-4-(4-{[-2-hydroxycyclopentyl]oxy}phenyl)-6-mercaptopyridine-3,5-dicarbonitrile

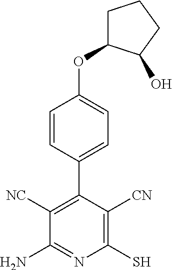

55 mg the compound of Example 16A (74% pure, 0.16 mmol), 9 mg (0.09 mmol) of 2-cyanothioacetamide and 37 μl of 4-methylmorpholine (0.34 mmol) in 0.5 ml of ethanol were heated under reflux for 1.5 h. A further 4 mg (0.04 mmol) of 2-cyanothioacetamide and 20 μl of 4-methylmorpholine were then added, and the mixture was heated under reflux for another 1.5 h. The reaction solution was then used directly, without work-up.

LC-MS (Method 9): Rₜ=1.71 min; MS (ESIpos): m/z=353 [M+H]⁺.

### Example 24A

### rac-2-Amino-6-mercapto-4-[4-(tetrahydrofuran-3-yloxy)phenyl]pyridine-3,5-dicarbonitrile

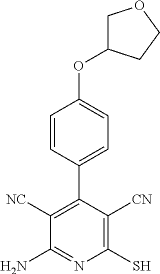

0.92 g (9.15 mmol) of 2-cyanothioacetamide and 0.92 ml (8.32 mmol) of 4-methylmorpholine were added to the reaction solution obtained in Example 17A, and the mixture was heated under relfux for 18 h. The precipitate formed was filtered off, washed with a little ethanol and dried.

Yield: 0.68 g (22% of theory)

LC-MS (Method 20): Rₜ=1.65 min; MS (ESIpos): m/z=339 [M+H]⁺

¹H-NMR (400 MHz, DMSO-d₆): δ=12.9 (br. s, 1H); 8.15-7.60 (br. s, 2H); 7.48 (d, 2H); 7.09 (d, 2H); 3.96-3.75 (m, 4H); 2.28 (m, 1H); 2.00 (m, 1H).

### Example 25A

### rac-2-Amino-4-[3-methoxy-4-(tetrahydrofuran-3-yloxy)phenyl]-6-sulfanylpyridine-3,5-dicarbonitrile

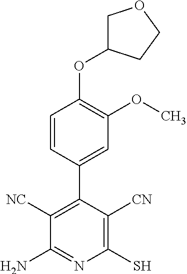

5.50 g (24.7 mmol) of the compound of Example 8A were dissolved in 120 ml of ethanol, 4.96 g (49.5 mmol) of 2-cyanothioacetamide and 5.44 ml (49.5 mmol) of 4-methylmorpholine were added and the mixture was heated under reflux for 3 h. After cooling, the precipitate formed was filtered off, the filtrate was freed from the solvent under reduced pressure, the residue was suspended in 100 ml of ethyl acetate and 100 ml 1 N hydrochloric acid and the mixture was sonicated for 10 minutes. The solid was filtered off, washed with water and diethyl ether and dried.

Yield: 1.71 g (19% of theory)

LC-MS (Method 3): Rₜ=1.65 min; MS (ESIpos): m/z=368 [M+H]⁺.

### Example 26A

### rac-2-Amino-4-[3-fluoro-4-(tetrahydrofuran-3-yloxy)phenyl]-6-sulfanylpyridine-3,5-dicarbonitrile

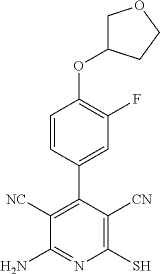

1.60 g (7.61 mmol) of the compound of Example 9A were dissolved in 37 ml of ethanol, 1.52 g (15.2 mmol) of 2-cyanothioacetamide and 1.67 ml (15.2 mmol) of 4-methylmorpholine were added and the mixture was heated under reflux for 3 h. After cooling, diethyl ether was added and the precipitate was filtered off, washed with diethyl ether and dried.

Yield: 0.68 g (23% of theory)

LC-MS (Method 3): Rₜ=1.79 min; MS (ESIpos): m/z=356 [M+H]⁺.

### Example 27A

### tert-Butyl 3-[4-(2-amino-3,5-dicyano-6-mercaptopyridin-4-yl)phenoxy]pyrrolidine-1-carboxylate

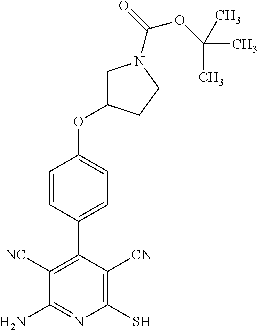

0.68 g (6.78 mmol) of 2-cyanothioacetamide and 0.69 ml (6.17 mmol) of 4-methylmorpholine were added to the reaction solution obtained in Example 18A, and the mixture was heated under reflux for 18 h. The solvent was then distilled off under reduced pressure, and the residue was processed directly, without any further purification.

Yield: 3.12 g (51% pure according to LC-MS, 59% of theory)

LC-MS (Method 3): Rₜ=2.34 min; MS (ESIpos): m/z=438 [M+H]⁺.

### Example 28A

### rac-trans-2-Amino-4-[4-({4-hydroxy-1-[(4-methyl phenyl)sulfonyl]pyrrolidin-3-yl}oxy)-phenyl]-6-mercaptopyridine-3,5-dicarbonitrile

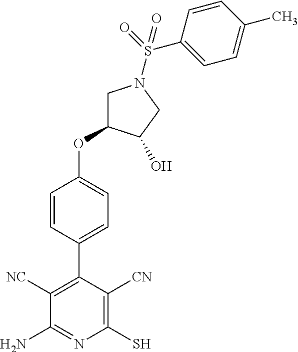

3.00 g (8.30 mmol) of the compound of Example 11A were dissolved in 86 ml of ethanol, 1.66 g (16.6 mmol) of 2-cyanothioacetamide and 1.82 ml (16.6 mmol) of 4-methylmorpholine were added and the mixture was stirred under reflux for 3 h. The mixture was then stirred into 150 ml of 1 N hydrochloric acid, the mixture was stirred at RT for 1 h, and the precipitate was then filtered off and taken up in ethyl acetate. The aqueous mother liquor was extracted with ethyl acetate, and the combined organic phases were washed with saturated sodium chloride solution, dried over magnesium sulfate and concentrated. The residue was chromatographed on silica gel using the mobile phase dichloromethane/methanol (50:1). This gave three product-containing fractions which were reacted without any further purification:

Fraction 1: 2.00 g (24% pure according to LC-MS, 11% of theory);

Fraction 2: 0.27 g (29% pure according to LC-MS, 1.8% of theory);

Fraction 3: 1.12 g (71% pure according to LC-MS, 19% of theory).

LC-MS (Method 5): Rₜ=2.78 min; MS (ESIpos): m/z=508 [M+H]⁺.

### Example 29A

### tert-Butyl 3-{4-[2-amino-6-({[2-(4-chlorophenyl)-1,3-thiazol-4-yl]methyl}thio)-3,5-dicyanopyridin-4-yl]phenoxy}pyrrolidine-1-carboxylate

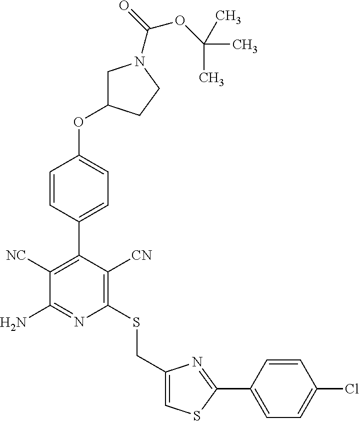

500 mg (0.58 mmol) of the compound of Example 27A and 156 mg (0.64 mmol) of 4-(chloromethyl)-2-(4-chlorophenyl)-1,3-thiazole were dissolved in 5.8 ml of DMF, 107 mg (1.28 mmol) of sodium bicarbonate were added and the mixture was stirred at 50° C. for 1 h. The mixture was then purified directly by preparative HPLC (column: YMC GEL ODS-AQ S-5, 15 μm; mobile phase gradient: acetonitrile/water 10:90→95:5).

Yield: 175 mg (47% of theory)

¹H-NMR (400 MHz, DMSO-d₆): δ=8.35-7.95 (br. s, 2H), 7.95 (d, 2H); 7.92 (s, 1H); 7.57 (d, 2H); 7.49 (d, 2H); 7.11 (d, 2H); 5.10 (br., 1H); 4.63 (s, 2H); 3.60 (m, 1H); 3.48-3.31 (m, 3H); 2.18 (m, 1H); 2.08 (m, 1H); 1.40 (s, 9H).

LC-MS (Method 3): Rₜ=1.40 min; MS (ESIpos): m/z=646 [M+H]⁺.

### Example 30A

### rac-trans-2-Chloro-6-({[2-(4-chlorophenyl)-1,3-thiazol-4-yl]methyl}sulfanyl)-4-(4-{[4-hydroxytetrahydrofuran-3-yl]oxy}phenyl)pyridine-3,5-dicarbonitrile

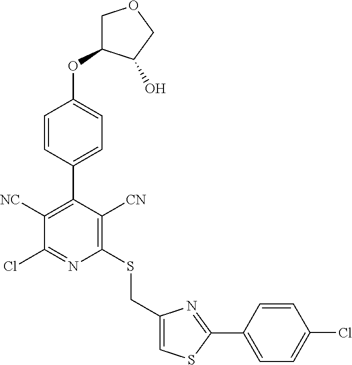

521 mg (0.93 mmol) of the compound of Example 24 were dissolved in 10 ml of 37% strength hydrochloric acid. At 0° C., 192 mg (2.78 mmol) of sodium nitrite were added to the mixture. The mixture was stirred initially at 0° C. for 1 h and then at room temperature overnight. The reaction mixture was purified directly by preparative HPLC (column: Reprosil C18, 10 μm; mobile phase A: water, mobile phase B: acetonitrile; gradient: 0.0 min 10% B→30 min 95% B→34 min 95% B→34.01 min 10% B→38 min 10% B; flow rate: 50 ml/min).

Yield: 406 mg (75% of theory)

¹H-NMR (400 MHz, DMSO-d₆): δ=7.95 (d, 2H); 7.75 (s, 1H); 7.64 (d, 2H); 7.57 (d, 2H); 7.23 (d, 2H); 4.79-4.75 (m, 3H); 4.25 (m, 1H); 4.08 (dd, 1H); 3.94 (dd, 1H); 3.87-3.70 (m, 2H); 3.60 (dd, 1H).

LC-MS (Method 3): Rₜ=3.01 min; MS (ESIpos): m/z=581 [M+H]⁺.

### Example 31A

### rac-3-[({6-Chloro-3,5-dicyano-4-[4-(tetrahydrofuran-3-yloxy)phenyl]pyridin-2-yl}sulfanyl)methyl]benzamide

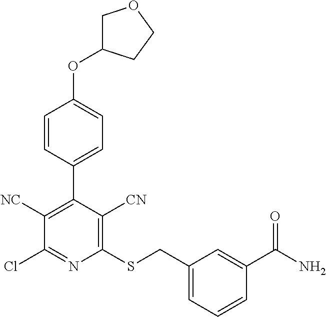

Under argon, 0.78 g (1.65 mmol) of the compound of Example 43 and 0.44 g (3.30 mmol) of copper(I) chloride were initially charged in 40 ml of acetonitrile, 0.44 ml (3.30 mmol) of isopentyl nitrite were added and the mixture was stirred at 60° C. for 4 h. After cooling, 3.3 ml of 1 N hydrochloric acid and 30 ml of water were added, and the mixture was extracted with ethyl acetate. The organic phase was washed with water and saturated aqueous sodium chloride solution, dried over magnesium sulfate and freed from the solvent on a rotary evaporator. The crude product obtained was purified by preparative HPLC (column: YMC GEL ODS-AQ S-5, 15 μm; mobile phase gradient: acetonitrile/water 10:90→95:5).

Yield: 0.19 g (22% of theory)

LC-MS (Method 3): Rₜ=2.33 min; MS (ESIpos): m/z=490 [M+H]⁺.

The compounds of the table below were prepared by the processes in the respective literature reference cited:

| | | Preparation | |
|---|---|---|---|
| Example | Structure | according to lit. | Analysis |
| 32A | 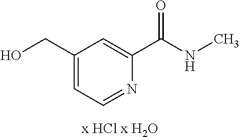 | U.S. Pat. No. 6,689,883-B1 | |
| 33A | 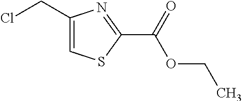 | C.B. Lee et al., J. Am. Chem. Soc. 123, 5249-5259 (2001) | |
| 34A | 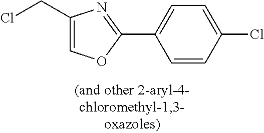 | I. Simiti, E. Chindris, Arch. Pharm. Ber. Dtsch. Pharm. Ges. 304, 425-429 (1971) | Rₜ = 3.78 min (Method 8); m/z = 228 [M]⁺ |
| 35A | 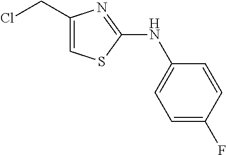 | WO 2006/027142-A1 | preparation in situ |

### Example 36A

### 4-(Chloromethyl)-N-methylpyridine-2-carboxamide hydrochloride

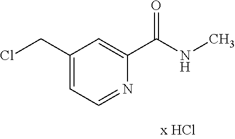

10 g (45.32 mmol) of the compound of Example 32A were suspended in 160 ml of dichloromethane and cooled to 0° C. After addition of 16.18 g (135.96 mmol) of thionyl chloride, the reaction mixture was warmed to RT and stirred at RT overnight. The mixture was then evaporated and the residue was dried under high vacuum.

Yield: 10 g (quant.)

LC-MS (Method 14): Rₜ=0.71 min; MS (ESIpos): m/z=185 [M+H]⁺

¹H-NMR (400 MHz, DMSO-d₆): δ=8.85-8.78 (m, 1H); 8.65 (d, 1H); 8.10 (s, 1H); 7.64 (d, 1H); 4.90 (s, 2H); 2.83 (d, 3H).

### Example 37A

### 2-(4-Chlorophenyl)-4,5-dimethyl-1,3-oxazole 3-oxide

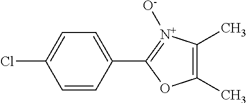

1.00 g (9.89 mmol) of diacetyl monoxime and 1.53 g (10.88 mmol) of 4-chlorobenzaldehyde were initially charged in 2 ml (34.94 mmol) of glacial acetic acid. With ice bath cooling of the reaction mixture, hydrogen chloride gas was then introduced for a period of 30 min. 10 ml of diethyl ether were then added to the reaction mixture. A precipitate was formed. This precipitate was filtered off with suction and washed twice with in each case 2 ml of diethyl ether. The precipitate was resuspended in about 5 ml of water, and the suspension was made basic using ammonia. The mixture was then extracted four times with in each case 10 ml of dichloromethane. The combined organic phases were dried over magnesium sulfate and the solvent was removed on a rotary evaporator. The residue was used without further purification for the next reaction.

Yield: 1.85 g (84% of theory)

LC-MS (Method 5): Rₜ=2.29 min; MS (ESIpos): m/z=224 [M+H]⁺.

### Example 38A

### 4-(Chloromethyl)-2-(4-chlorophenyl)-5-methyl-1,3-oxazole

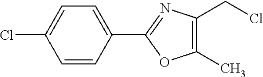

1.00 g (4.47 mmol) of the compound of Example 37A were initially charged in 15 ml of chloroform, and 1.5 ml (16.10 mmol) of phosphoryl chloride were added carefully. With stirring, the reaction mixture was heated at reflux for 30 min. The mixture was then cooled to 0° C. and made slightly basic by addition of ammonia. The mixture was then extracted three times with in each case 20 ml of ethyl acetate. The combined organic phases were washed twice with in each case 5 ml of water and then dried over magnesium sulfate. The solvent was removed on a rotary evaporator. The residue was used without further purification for the next steps.

Yield: 1.33 g (96% of theory, 78% pure)

¹H-NMR (400 MHz, DMSO-d₆): δ=7.95 (d, 2H); 7.60 (d, 2H); 4.77 (s, 2H); 2.44 (s, 3H).

LC-MS (Method 3): Rₜ=2.80 min; MS (ESIpos): m/z=242 [M+H]⁺.

### Example 39A

### rac-trans-2-Amino-4-(4-{[4-{[tert-butyl(dimethyl)silyl]oxy}tetrahydrofuran-3-yl]oxy}-phenyl)-6-({[2-(4-chlorophenyl)-1,3-thiazol-4-yl]methyl}thio)pyridine-3,5-dicarbonitrile

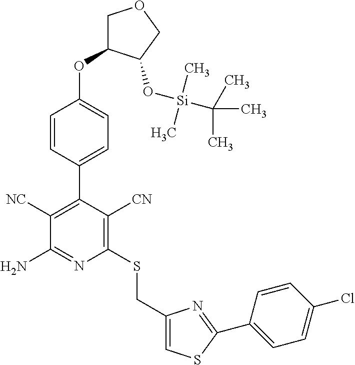

190 mg (0.34 mmol) of the compound of Example 21A and 91.5 mg (0.38 mmol) of 4-(chloromethyl)-2-(4-chlorophenyl)-1,3-thiazole were dissolved in 3.4 ml of DMF, 94.1 mg (0.68 mmol) of potassium carbonate were added and the mixture was stirred at 50° C. for 1 h. The mixture was then purified directly by preparative HPLC (column: YMC GEL ODS-AQ S-5, 15 μm; mobile phase gradient: acetonitrile/water 10:90→95:5).

Yield: 196 mg (85% of theory)

¹H-NMR (400 MHz, DMSO-d₆): δ=8.30-7.90 (br. s, 2H); 7.96 (s, 1H); 7.92 (d, 2H); 7.50 (d, 2H); 4.81 (d, 1H); 4.64 (s, 2H); 4.41 (br. t, 1H); 4.09 (dd, 1H); 4.01 (dd, 1H); 3.79 (d, 1H); 3.54 (dd, 1H); 0.88 (s, 9H); 0.08 (s, 3H); 0.06 (s, 3H).

HPLC (Method 19): Rₜ=6.33 min; MS (ESIpos): m/z=676 [M+H]⁺.

### Example 40A

### rac-trans-2-Amino-6-(benzylthio)-4-[4-({4-hydroxy-1-[(4-methylphenyl)sulfonyl]-pyrrolidin-3-yl}oxy)phenyl]pyridine-3,5-dicarbonitrile

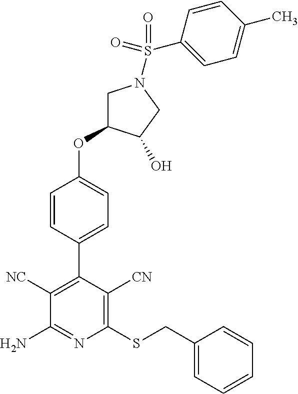

2.00 g (1.14 mmol) of Product Fraction 1 from Example 28A together with 0.21 g (1.26 mmol) of benzyl bromide were dissolved in 11.4 ml of DMF, 0.35 g (2.51 mmol) of potassium carbonate was added and the mixture was stirred at room temperature for 1 h. The mixture was then purified directly by preparative HPLC (column: YMC GEL ODS-AQ S-5, 15 μm; mobile phase gradient: acetonitrile/water 10:90→95:5). This gave 270 mg of the target compound (90% pure according to LC-MS, 36% of theory), 50 mg of which were re-purified by another preparative HPLC. This afforded 32 mg (4.5% of theory) of the pure title compound.

¹H-NMR (400 MHz, DMSO-d₆): δ=8.35-7.95 (br. s, 2H); 7.61 (d, 2H); 7.51 (d, 2H); 7.42 (d, 2H); 7.39-7.26 (m, 5H); 6.78 (d, 2H); 5.60 (d, 1H); 4.63 (d, 1H); 4.50 (s, 2H); 4.15 (br. t, 1H); 3.59 (dd, 1H); 3.40-3.30 (m, 2H); 3.18 (m, 1H); 2.40 (s, 3H).

LC-MS (Method 20): Rₜ=2.55 min; MS (ESIpos): m/z=598 [M+H]⁺.

### Example 41A

### 2-Amino-6-(phenylsulfanyl)-4-[4-(tetrahydrofuran-3-yloxy)phenyl]pyridine-3,5-dicarbonitrile

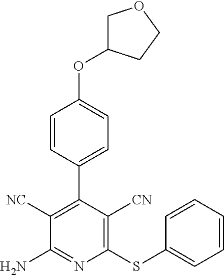

7.0 g (36.4 mmol) of the compound of Example 7A, 4.81 g (72.8 mmol) of malononitrile and 4.0 g (36.4 mmol) of thiophenol were initially charged in 65 ml of ethanol, 0.1 ml of triethylamine was added and the mixture was stirred under reflux overnight. The precipitate formed was filtered off, washed with a little cold ethanol and dried under reduced pressure.

Yield: 6.28 g (41% of theory)

LC-MS (Method 3): Rₜ=2.51 min; MS (ESIpos): m/z=415 [M+H]⁺.

### Example 42A and Example 43A

### ent-[4-(Tetrahydrofuran-3-yloxy)phenyl]methanol

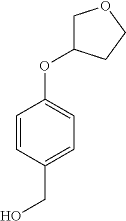

20 g (104.049 mmol) of the compound of Example 7A were dissolved in 350 ml of THF, and at 0° C. 83 ml (83.239 mmol) of a 1 M solution of lithium aluminum hydride in THF were added dropwise. After one hour of stirring at 0° C., 260 ml of ethyl acetate, 10 ml of water, 10 ml of 1 N aqueous sodium hydroxide solution and another 21 ml of water were added in succession. The precipitate was filtered off and the filtrate was concentrated on a rotary evaporator. The title compound was separated into the enantiomers by preparative HPLC of the residue on a chiral phase [column: Daicel Chiralpak AS-H, 250 mm×20 mm; mobile phase: isohexane/isopropanol 70:30 (v/v); flow rate: 15 ml/min; temperature: 30° C.; UV detection: 220 nm]:

### Example 42A

### Enantiomer 1

Yield: 9.9 g (chem. purity 65%, 32% of theory, >99% ee)

Rₜ=7.60 min.

[column: Daicel Chiralpak AS-H, 250 mm×4.6 mm; mobile phase: isohexane/isopropanol 70:30 (v/v); flow rate: 1 ml/min; temperature: 35° C.; UV detection: 220 nm]

LC-MS (Method 3): Rₜ=1.22 min; MS (ESIpos): m/z=177 [M−H₂O+H]⁺.

### Example 43A

### Enantiomer 2

Yield: 8.8 g (chem. purity 65%, 28% of theory, >98% ee)

Rₜ=8.77 min.

[column: Daicel Chiralpak AS-H, 250 mm×4.6 mm; mobile phase: isohexane/isopropanol 70:30 (v/v); flow rate: 1 ml/min; temperature: 35° C.; UV detection: 220 nm]

LC-MS (Method 3): Rₜ=1.22 min; MS (ESIpos): m/z=177 [M−H₂O+H]⁺.

### Example 44A

### ent-4-(Tetrahydrofuran-3-yloxy)benzaldehyde

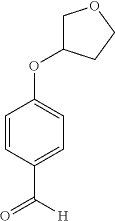

9.9 g (purity 65%, 33.13 mmol) of the compound of Example 42A (enantiomer 1) were dissolved in 85 ml of methanol, and 23.042 g (265.043 mmol) of manganese dioxide were added. The mixture was stirred at 40° C. overnight. The reaction mixture was then filtered through silica gel, the filtrate was concentrated and the residue was chromatographed on silica gel (mobile phase: dichloromethane/THF initially 40:1, then 20:1).

Yield: 5.74 g (purity 87%, 78% of theory)

LC-MS (Method 14): Rₜ=0.84 min; MS (ESIpos): m/z=193 [M+H]⁺.

### Example 45A

### ent-2-Amino-6-mercapto-4-[4-(tetrahydrofuran-3-yloxy)phenyl]pyridine-3,5-dicarbonitrile

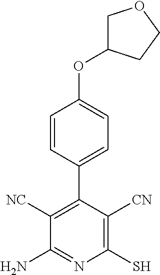

6.275 g (32.645 mmol) of the compound of Example 44A, 6.538 g (65.291 mmol) of 2-cyanothioacetamide and 6.604 g (65.291 mmol) of 4-methylmorpholine were dissolved in 80 ml of ethanol. The reaction mixture was stirred under reflux for 4 h and then cooled to 0° C. The precipitated solid was filtered off with suction, washed with a little ice-cold ethanol and dried under high vacuum.

Yield: 2.72 g (25% of theory)

LC-MS (Method 3): Rₜ=1.69 min; MS (ESIpos): m/z=339 [M+H]⁺.

### WORKING EXAMPLES

### Example 1

### rac-trans-2-Amino-4-(4-{[-2-hydroxycyclopentyl]oxy}phenyl)-6-[(pyridin-3-ylmethyl)-thio]pyridine-3,5-dicarbonitrile

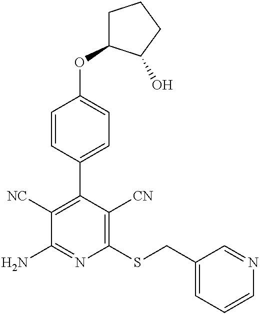

A solution of 200 mg (0.568 mmol) of the compound of Example 19A in 2 ml of DMF together with 107 mg (0.624 mmol) of 3-chloromethylpyridine hydrochloride and 157 mg (1.135 mmol) of potassium carbonate was stirred at 50° C. overnight. After addition of 0.7 ml of 5 N acetic acid, the reaction mixture was purified directly by preparative HPLC.

Yield: 174 mg (69% of theory)

LC-MS (Method 9): Rₜ=1.84 min; MS (ESIpos): m/z=444 [M+H]⁺

¹H-NMR (400 MHz, DMSO-d₆): δ=8.78 (d, 1H); 8.48-8.41 (m, 1H); 8.41-7.70 (br. s, 2H); 7.97-7.90 (m, 1H); 7.45 (d, 2H); 7.38-7.30 (m, 1H); 7.08 (d, 2H); 5.03 (d, 1H); 4.56-4.44 (m, 3H); 4.12-4.04 (m, 1H); 2.22-2.09 (m, 1H); 1.93-1.82 (m, 1H); 1.83-1.59 (m, 3H); 1.59-1.50 (m, 1H).

The compounds of the table below were prepared analogously to the procedure for Example 1 from the starting materials stated and the appropriate alkylation components. The alkylation components are commercially available or have been described before, or they can be prepared by customary methods known to the person skilled in the art.

| | | Starting | |
|---|---|---|---|
| | | material; | |
| Example | Structure | yield | Analysis |
| 2 | 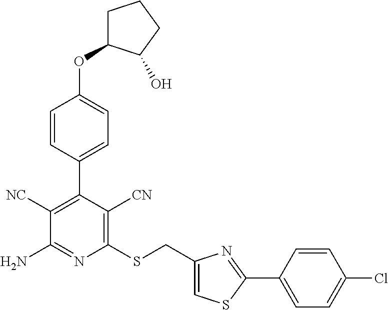 | 19A; 32% | LC-MS (Method 2): Rₜ = 2.78 min; MS (ESIpos): m/z = 560 [M + H]⁺ |
| 3 | 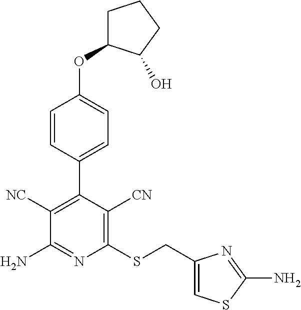 | 19A; 7% | LC-MS (Method 9): Rₜ = 1.78 min; MS (ESIpos): m/z = 465 [M + H]⁺ |
| 4 | 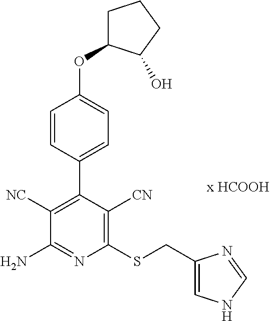 | 19A; 27% | LC-MS (Method 9): Rₜ = 1.31 min; MS (ESIpos): m/z = 433 [M + H]⁺ |
| 5 | 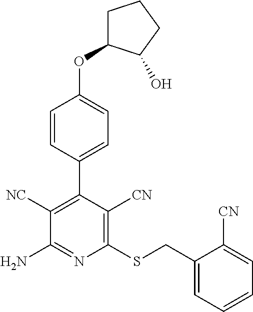 | 19A; 54% (cryst. from ethanol/ water) | LC-MS (Method 2): Rₜ = 2.33 min; MS (ESIpos): m/z = 468 [M + H]⁺ |
| 6 | 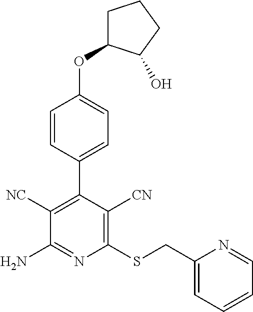 | 19A; 45% | LC-MS (Method 9): Rₜ = 2.10 min; MS (ESIpos): m/z = 444 [M + H]⁺ |
| 7 | 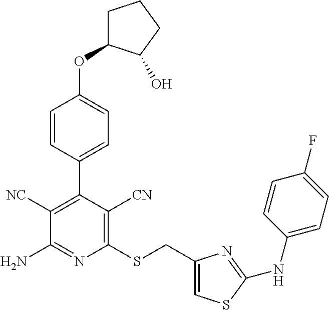 | 19A; 21% | LC-MS (Method 2): Rₜ = 2.44 min; MS (ESIpos): m/z = 559 [M + H]⁺ |
| 8 | 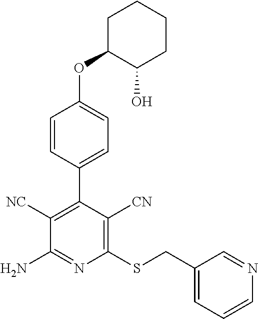 | 20A; 28% | LC-MS (Method 9): Rₜ= 1.94 min; MS (ESIpos): m/z = 458 [M + H]⁺ |
| 9 | 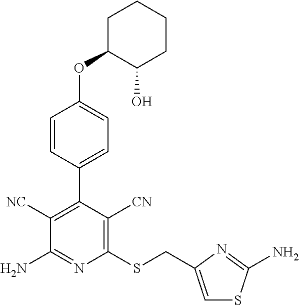 | 20A; 13% | LC-MS (Method 2): Rₜ = 1.72 min; MS (ESIpos): m/z = 479 [M + H]⁺ |
| 10 | 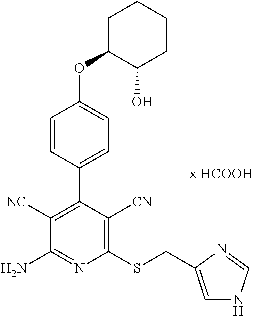 | 20A; 32% | LC-MS (Method 9): Rₜ = 1.53 min; MS (ESIpos): m/z = 447 [M + H]⁺ |
| 11 | 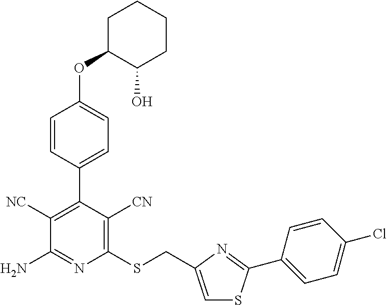 | 20A; 32% (after two HPLC purifications) | LC-MS (Method 9): Rₜ = 2.99 min; MS (ESIpos): m/z = 574 [M + H]⁺ |
| 12 | 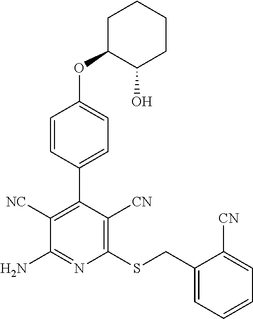 | 20A; 41% | LC-MS (Method 2): Rₜ = 2.40 min; MS (ESIpos): m/z = 482 [M + H]⁺ |
| 13 | 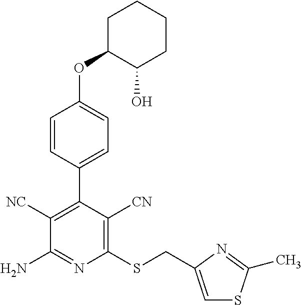 | 20A; 73% | LC-MS (Method 9): Rₜ = 2.36 min; MS (ESIpos): m/z = 478 [M + H]⁺ |
| 14 | 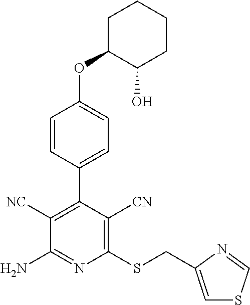 | 20A; 77% | LC-MS (Method 2): Rₜ = 2.11 min; MS (ESIpos): m/z = 464 [M + H]⁺ |
| 15 | 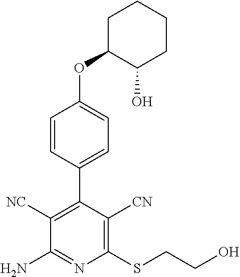 | 20A; 51% | LC-MS (Method 2): Rₜ = 1.87 min; MS (ESIpos): m/z = 411 [M + H]⁺ |
| 16 | 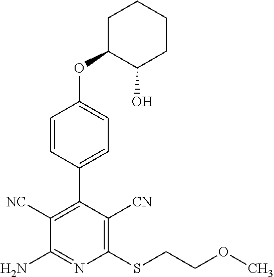 | 20A; 45% | LC-MS (Method 2): Rₜ = 2.14 min; MS (ESIpos): m/z = 425 [M + H]⁺ |
| 17 | 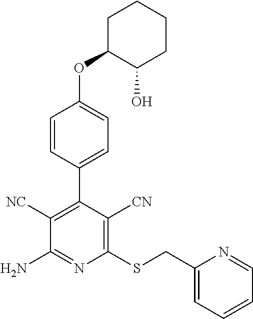 | 20A; 79% | LC-MS (Method 3): Rₜ = 2.21 min; MS (ESIpos): m/z = 458 [M + H]⁺ |
| 18 | 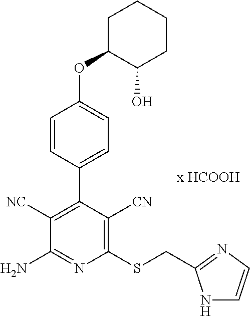 | 20A; 40% | LC-MS (Method 9): Rₜ = 1.50 min; MS (ESIpos): m/z = 447 [M + H]⁺ |
| 19 | 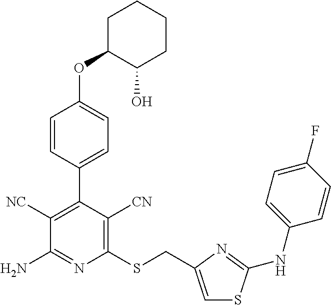 | 20A; 32% | LC-MS (Method 9): Rₜ = 2.69 min; MS (ESIpos): m/z = 573 [M + H]⁺ |

### Example 20 and Example 21

### ent-trans-2-Amino-6-({[2-(4-chlorophenyl)-1,3-thiazol-4-yl]methyl}sulfanyl)-4-(4-{[2-hydroxycyclohexyl]oxy}phenyl)pyridine-3,5-dicarbonitrile

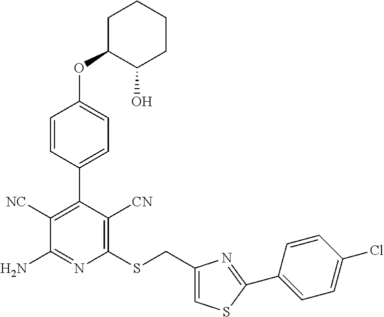

By preparative HPLC on a chiral phase, the compound from Example 11 was separated into the enantiomers [instrument type: Agilent 1100 with DAD detection; column: Daicel Chiralpak AD-H, 5 μm, 250 mm×20 mm; mobile phase: isohexane/methanol/isopropanol (3:1:1); flow rate: 20 ml/min; temperature: 24° C.; UV detection: 250 nm]:

### Example 20

### Enantiomer 1

Rₜ=5.305 min.

[column: Daicel Chiralpak AD-H, 5 μm, 250 mm×4 mm; mobile phase: isohexane/methanol/isopropanol (4:1:1); flow rate: 1.5 ml/min; temperature: 30° C.; UV detection: 290 nm].

### Example 21

### Enantiomer 2

Rₜ=6.441 min.

[column: Daicel Chiralpak AD-H, 5 μm, 250 mm×4 mm; mobile phase: isohexane/methanol/isopropanol (4:1:1); flow rate: 1.5 ml/min; temperature: 30° C.; UV detection: 290 nm].

### Example 22 and Example 23

### ent-trans-2-Amino-6-[(2-cyanobenzyl)sulfanyl]-4-(4-{[2-hydroxycyclohexyl]oxy}-phenyl)pyridine-3,5-dicarbonitrile

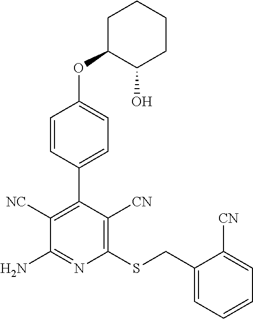

By preparative HPLC on a chiral phase, the compound from Example 12 was separated into the enantiomers [instrument type: Agilent 1100 with DAD detection; column: Daicel Chiralcel OD-H, 5 μm, 250 mm×20 mm; mobile phase: isohexane/ethanol (65:35); flow rate: 20 ml/min; temperature: 24° C.; UV detection: 220 nm]:

### Example 22

### Enantiomer 1

Rₜ=13.416 min.

[column: Daicel Chiralpak AD-H, 5 μm, 250 mm×4 mm; mobile phase: isohexane/ethanol (3:2); flow rate: 1.0 ml/min; temperature: 30° C.; UV detection: 290 nm].

### Example 23

### Enantiomer 2

Rₜ=15.233 min.

[column: Daicel Chiralpak AD-H, 5 μm, 250 mm×4 mm; mobile phase: isohexane/ethanol (3:2); flow rate: 1.0 ml/min; temperature: 30° C.; UV detection: 290 nm].

### Example 24

### rac-trans-2-Amino-6-({[2-(4-chlorophenyl)-1,3-thiazol-4-yl]methyl}thio)-4-(4-{[4-hydroxytetrahydrofuran-3-yl]oxy}phenyl)pyridine-3,5-dicarbonitrile

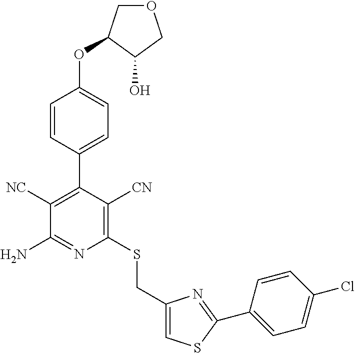

170 mg (0.25 mmol) of the compound of Example 39A were dissolved in 2.5 ml of THF, and 0.5 ml of a 1 M solution of tetra-n-butylammonium fluoride in THF was added. The reaction mixture was stirred at RT for 1 h. The mixture was then stirred into 20 ml of water and extracted with 20 ml of ethyl acetate. The organic phase was washed with saturated aqueous sodium chloride solution, dried over magnesium sulfate, filtered and concentrated.

Yield: 131 mg (93% of theory)

¹H-NMR (500 MHz, DMSO-d₆): δ=8.45-7.90 (br. s, 1H); 7.95 (d, 2H); 7.92 (s, 1H); 7.57 (d, 2H); 7.50 (d, 2H); 7.15 (d, 2H); 5.54 (d, 1H); 4.74 (d, 1H); 4.64 (s, 2H); 4.24 (br. t, 1H); 4.08 (dd, 1H); 3.93 (dd, 1H); 3.80 (d, 1H); 3.60 (dd, 1H).

HPLC (Method 19): Rₜ=4.90 min; MS (DCI/NH₃): m/z=562 [M+H]⁺.

### Example 25 and Example 26

### ent-trans-2-Amino-6-({[2-(4-chlorophenyl)-1,3-thiazol-4-yl]methyl}thio)-4-(4-{[4-hydroxytetrahydrofuran-3-yl]oxy}phenyl)pyridine-3,5-dicarbonitrile

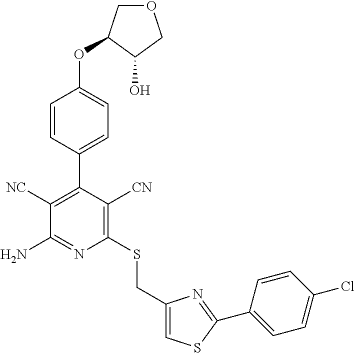

By preparative HPLC on a chiral phase, the compound from Example 24 was separated into the enantiomers [instrument type: Agilent 1100 with DAD detection; column: Daicel Chiralpak AD-H, 5 μm, 250 mm×20 mm; mobile phase: isohexane/isopropanol (7:3); flow rate: 15 ml/min; temperature: 24° C.; UV detection: 290 nm]:

### Example 25

### Enantiomer 1

Rₜ=12.41 min.

[column: Daicel Chiralpak AD-H, 5 μm, 250 mm×4 mm; mobile phase: isohexane/isopropanol (7:3); flow rate: 1.0 ml/min; temperature: 30° C.; UV detection: 290 nm].

### Example 26

### Enantiomer 2

Rₜ=14.49 min.

[column: Daicel Chiralpak AD-H, 5 μm, 250 mm×4 mm; mobile phase: isohexane/isopropanol (7:3); flow rate: 1.0 ml/min; temperature: 30° C.; UV detection: 290 nm].

### Example 27

### rac-trans-2-({[2-(4-Chlorophenyl)-1,3-thiazol-4-yl]methyl}sulfanyl)-6-(3-hydroxyazetidin-1-yl)-4-(4-{[4-hydroxytetrahydrofuran-3-yl]oxy}phenyl)pyridine-3,5-dicarbonitrile

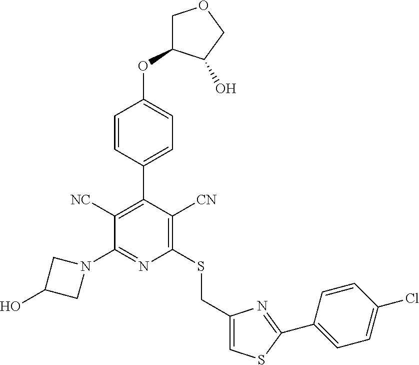

415 mg (0.71 mmol) of the compound of Example 30A were dissolved in 10 ml of THF, 156 mg (1.43 mmol) of 3-hydroxyazetidine hydrochloride and 185 mg (1.43 mmol) of N,N-diisopropylethylamine were added and the mixture was stirred at RT overnight. The reaction mixture was purified directly by preparative HPLC (column: Reprosil C18, 10 μm; mobile phase A: water, mobile phase B: acetonitrile; gradient: 0.0 min 10% B→30 min 95% B→34 min 95% B→34.01 min 10% B→38 min 10% B; flow rate: 50 ml/min).

Yield: 183 mg (41% of theory)

LC-MS (Method 3): Rₜ=2.67 min; MS (ESIpos): m/z=618 [M+H]⁺.

### Example 28 and Example 29

### ent-trans-2-({[2-(4-Chlorophenyl)-1,3-thiazol-4-yl]methyl}sulfanyl)-6-(3-hydroxyazetidin-1-yl)-4-(4-{[4-hydroxytetrahydrofuran-3-yl]oxy}phenyl)pyridine-3,5-dicarbonitrile

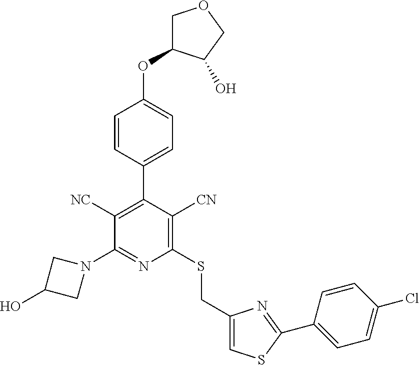

By preparative HPLC on a chiral phase, the compound from Example 27 was separated into the enantiomers [column: Chiralpak IC, 250 mm×20 mm; mobile phase: methyl tert-butyl ether/acetonitrile (7:3); flow rate: 15 ml/min; temperature: 30° C.; UV detection: 220 nm]:

### Example 28

### Enantiomer 1

Rₜ=6.01 min.

[column: Chiralpak IC, 250 mm×4.6 mm; mobile phase: methyl tert-butyl ether/acetonitrile (7:3); flow rate: 1.0 ml/min; temperature: 25° C.; UV detection: 220 nm].

¹H-NMR (400 MHz, DMSO-d₆): δ=7.95 (d, 2H); 7.68 (s, 1H); 7.57 (d, 2H); 7.49 (d, 2H); 7.15 (d, 2H); 5.88 (d, 1H); 5.54 (d, 1H); 4.74 (d, 1H); 4.71-4.54 (m, 5H); 4.24 (br. s, 1H); 4.16 (br. d, 2H); 4.08 (dd, 1H); 3.93 (dd, 1H); 3.79 (d, 1H); 3.60 (dd, 1H).

### Example 29

### Enantiomer 2

Rₜ=7.46 min.

[column: Chiralpak IC, 250 mm×4.6 mm; mobile phase: methyl tert-butyl ether/acetonitrile (7:3); flow rate: 1.0 ml/min; temperature: 25° C.; UV detection: 220 nm].

### Example 30

### rac-trans-2-Amino-4-(4-{[4-hydroxytetrahydrofuran-3-yl]oxy}phenyl)-6-[(pyridin-3-ylmethyl)thio]pyridine-3,5-dicarbonitrile

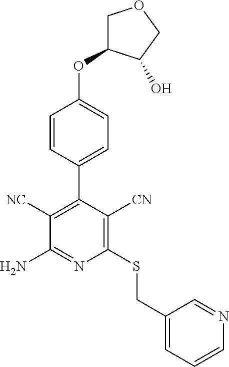

300 mg (0.42 mmol) of the crude product from Example 22A and 76.4 mg (0.47 mmol) of 3-picolyl chloride hydrochloride were dissolved in 4.2 ml of DMF, 128 mg (0.93 mmol) of potassium carbonate were added and the mixture was stirred at 50° C. for 1 h. The mixture was purified directly by preparative HPLC (Method 18). For further purification, the product was chromatographed once more on silica gel using the mobile phase dichloromethane/methanol (50:1).

Yield: 45 mg (23% of theory)

¹H-NMR (400 MHz, DMSO-d₆): δ=8.78 (s, 1H); 8.45 (d, 1H); 8.40-7.90 (br. s, 2H); 7.95 (d, 1H); 7.50 (d, 2H); 7.34 (dd, 1H); 7.14 (dd, 2H); 5.54 (d, 1H); 4.74 (d, 1H); 4.49 (s, 2H); 4.23 (br. t, 1H); 4.08 (dd, 1H); 3.92 (dd, 1H); 3.80 (d, 1H); 3.60 (dd, 1H).

LC-MS (Method 2): Rₜ=1.41 min; MS (ESIpos): m/z=445 [M+H]⁺.

### Example 31

### rac-trans-2-Amino-4-(4-{[4-hydroxytetrahydrofuran-3-yl]oxy}phenyl)-6-[(pyridin-2-ylmethyl)thio]pyridine-3,5-dicarbonitrile

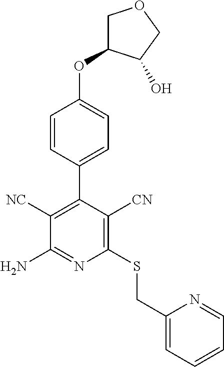

The title compound was prepared analogously to the procedure of Example 30 from the appropriate starting materials.

Yield: 53% of theory

¹H-NMR (400 MHz, DMSO-d₆): δ=8.53 (d, 1H); 8.40-7.90 (br. s, 2H); 7.76 (dt, 1H); 7.65 (d, 1H); 7.50 (dd, 1H); 7.29 (t, 1H); 7.15 (dd, 2H); 5.54 (d, 1H); 4.75 (d, 1H); 4.61 (s, 2H); 4.24 (br. t, 1H); 4.08 (dd, 1H); 3.93 (dd, 1H); 3.80 (d, 1H); 3.60 (dd, 1H).

LC-MS (Method 3): Rₜ=1.86 min; MS (ESIpos): m/z=445 [M+H]⁺.

### Example 32 and Example 33

### ent-trans-2-Amino-4-(4-{[4-hydroxytetrahydrofuran-3-yl]oxy}phenyl)-6-[(pyridin-2-ylmethyl)thio]pyridine-3,5-dicarbonitrile

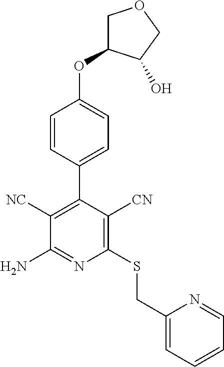

By preparative HPLC on a chiral phase, the compound from Example 31 was separated into the enantiomers [instrument type: Agilent 1100 with DAD detection; column: Daicel Chiralpak AD-H, 5 μm, 250 mm×20 mm; mobile phase: isohexane/ethanol (3:2); flow rate: 20 ml/min; temperature: 24° C.; UV detection: 290 nm]:

### Example 32

### Enantiomer 1

Rₜ=14.12 min.

[column: Daicel Chiralpak AD-H, 5 μm, 250 mm×4 mm; mobile phase: isohexane/ethanol (3:2); flow rate: 1.0 ml/min; temperature: 30° C.; UV detection: 290 nm].

### Example 33

### Enantiomer 2

Rₜ=21.40 min.

[column: Daicel Chiralpak AD-H, 5 μm, 250 mm×4 mm; mobile phase: isohexane/ethanol (3:2); flow rate: 1.0 ml/min; temperature: 30° C.; UV detection: 290 nm].

### Example 34

### rac-trans-2-Amino-6-[({2-[(4-fluorophenyl)amino]-1,3-thiazol-4-yl}methyl)thio]-4-(4-{[4-hydroxytetrahydrofuran-3-yl]oxy}phenyl)pyridine-3,5-dicarbonitrile

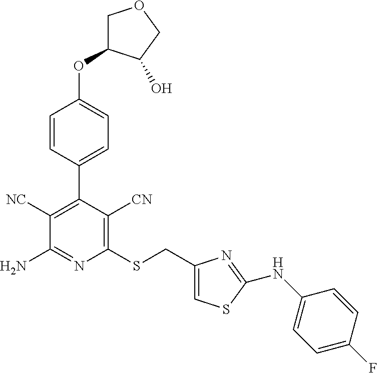

The title compound was prepared analogously to the procedure of Example 30 from starting materials 22A and 35A.

Yield: 42% of theory

¹H-NMR (400 MHz, DMSO-d₆): δ=10.2 (s, 1H); 8.30-7.90 (br. s, 2H); 7.62 (dd, 2H); 7.50 (d, 2H); 7.14 (m, 4H); 6.97 (s, 1H); 5.54 (d, 1H); 4.75 (d, 1H); 4.45 (s, 2H); 4.24 (br. t, 1H); 4.08 (dd, 1H); 3.93 (dd, 1H); 3.80 (d, 1H); 3.60 (dd, 1H).

LC-MS (Method 3): Rₜ=2.47 min; MS (ESIpos): m/z=560 [M+H]⁺.

### Example 35 and Example 36

### ent-trans-2-Amino-6-[({2-[(4-fluorophenyl)amino]-1,3-thiazol-4-yl}methyl)thio]-4-(4-{[4-hydroxytetrahydrofuran-3-yl]oxy}phenyl)pyridine-3,5-dicarbonitrile

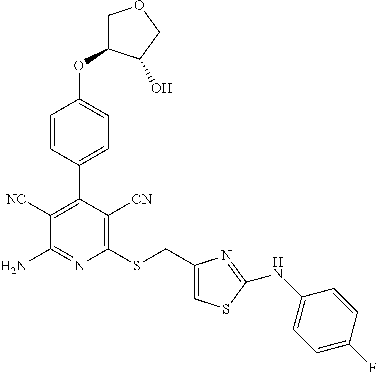

By preparative HPLC on a chiral phase, the compound from Example 34 was separated into the enantiomers [instrument type: Agilent 1100 with DAD detection; column: Daicel Chiralpak AD-H, 5 μm, 250 mm×20 mm; mobile phase: isohexane/isopropanol (3:2); flow rate: 20 ml/min; temperature: 24° C.; UV detection: 290 nm]:

### Example 35

### Enantiomer 1

Rₜ=21.42 min.

[column: Daicel Chiralpak AD-H, 5 μm, 250 mm×4 mm; mobile phase: isohexane/ethanol (3:2); flow rate: 1.0 ml/min; temperature: 30° C.; UV detection: 290 nm].

### Example 36

### Enantiomer 2

Rₜ=28.31 min.

[column: Daicel Chiralpak AD-H, 5 μm, 250 mm×4 mm; mobile phase: isohexane/ethanol (3:2); flow rate: 1.0 ml/min; temperature: 30° C.; UV detection: 290 nm].

### Example 37

### rac-trans-2-Amino-6-[(2-fluoroethyl)thio]-4-(4-{[4-hydroxytetrahydrofuran-3-yl]oxy}-phenyl)pyridine-3,5-dicarbonitrile

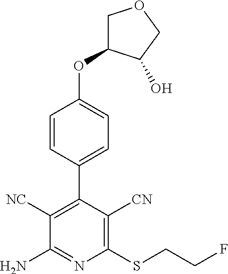

The title compound was prepared analogously to the procedure of Example 30 from the appropriate starting materials.

Yield: 10% of theory

¹H-NMR (400 MHz, DMSO-d₆): δ=8.25-7.85 (br. s, 2H); 7.51 (d, 2H); 7.16 (d, 2H); 5.54 (d, 1H); 4.76 (d, 1H); 4.72 (t, 2H); 4.60 (t, 2H); 4.25 (br. t, 1H); 4.08 (dd, 1H); 3.93 (dd, 1H); 3.80 (d, 1H); 3.64-3.53 (m, 3H).

LC-MS (Method 4): Rₜ=3.02 min; MS (ESIpos): m/z=401 [M+H]⁺.

### Example 38

### rac-trans-2-Amino-6-[(2,2-difluoroethyl)thio]-4-(4-{[4-hydroxytetrahydrofuran-3-yl]oxy}phenyl)pyridine-3,5-dicarbonitrile

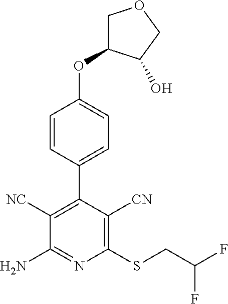

The title compound was prepared analogously to the procedure of Example 30 from the appropriate starting materials.

Yield: 11% of theory

¹H-NMR (400 MHz, DMSO-d₆): δ=8.40-7.90 (br. s, 2H); 7.53 (d, 2H); 7.17 (d, 2H); 6.32 (tt, 1H); 5.55 (d, 1H); 4.76 (d, 1H); 4.25 (br. t, 1H); 4.09 (dd, 1H); 3.93 (dd, 1H); 3.87-3.74 (m, 3H); 3.60 (dd, 1H).

LC-MS (Method 4): Rₜ=3.13 min; MS (ESIpos): m/z=419 [M+H]⁺.

### Example 39

### rac-trans-2-Amino-6-({[2-(4-chlorophenyl)-1,3-oxazol-4-yl]methyl}thio)-4-(4-{[4-hydroxytetrahydrofuran-3-yl]oxy}phenyl)pyridine-3,5-dicarbonitrile

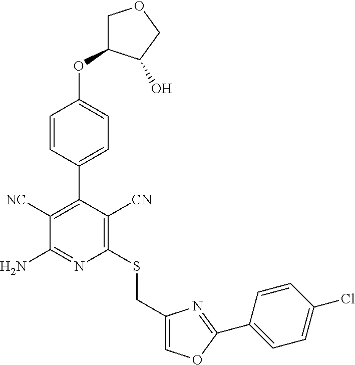

The title compound was prepared analogously to the procedure of Example 30 from starting materials 22A and 34A.

Yield: 14% of theory

¹H-NMR (500 MHz, DMSO-d₆): δ=8.36 (s, 1H); 8.30-7.90 (br. s, 1H); 7.97 (d, 2H); 7.60 (d, 2H); 7.49 (d, 2H); 7.15 (d, 2H); 5.54 (d, 1H); 4.74 (d, 1H); 4.42 (s, 2H); 4.24 (br. t, 1H); 4.08 (dd, 1H); 3.93 (dd, 1H); 3.80 (d, 1H); 3.60 (dd, 1H).

LC-MS (Method 3): Rₜ=2.21 min; MS (ESIpos): m/z=546 [M+H]⁺.

### Example 40

### rac-trans-2-Amino-6-({[2-(4-chlorophenyl)-5-methyl-1,3-oxazol-4-yl]methyl}thio)-4-(4-{[4-hydroxytetrahydrofuran-3-yl]oxy}phenyl)pyridine-3,5-dicarbonitrile

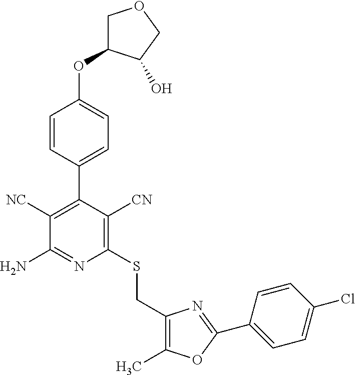

The title compound was prepared analogously to the procedure of Example 30 from starting materials 22A and 38A.

Yield: 19% of theory

¹H-NMR (500 MHz, DMSO-d₆): δ=8.22-7.95 (br. s, 1H); 7.92 (d, 2H); 7.58 (d, 2H); 7.49 (d, 2H); 7.15 (d, 2H); 5.54 (d, 1H); 4.75 (d, 1H); 4.51 (s, 2H); 4.24 (br. t, 1H); 4.08 (dd, 1H); 3.93 (dd, 1H); 3.80 (d, 1H); 3.60 (dd, 1H); 2.47 (s, 3H).

LC-MS (Method 6): Rₜ=2.33 min; MS (ESIpos): m/z=560 [M+H]⁺.

### Example 41

### rac-cis-2-Amino-4-(4-{[2-hydroxycyclopentyl]oxy}phenyl)-6-[(pyridin-3-ylmethyl)sulfanyl]pyridine-3,5-dicarbonitrile

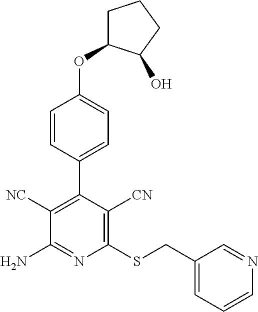

1 ml of DMF, 43 mg (0.25 mmol) of 3-chloromethylpyridine hydrochloride and 70 mg (0.5 mmol) of potassium carbonate were added to the reaction solution obtained in Example 23A, and the mixture was stirred at 50° C. overnight. 0.2 ml of 5 N acetic acid was then added, and the solution was purified directly by preparative HPLC. The product-containing fractions were purified once more by preparative HPLC. The solid obtained (21.6 mg) was dissolved in hot ethanol, water was added and the mixture was partially concentrated. The resulting precipitate was filtered off with suction, washed with water and dried under high vacuum.

Yield: 6.8 mg (9% of theory)

LC-MS (Method 3): Rₜ=1.84 min; MS (ESIpos): m/z=444 [M+H]⁺

¹H-NMR (400 MHz, DMSO-d₆): δ=8.79 (s, 1H); 8.49-8.40 (m, 1H); 8.40-7.70 (br. s, 2H); 7.98-7.92 (m, 1H); 7.45 (d, 2H); 7.40-7.32 (m, 1H); 7.10 (d, 2H); 4.75-4.60 (br., 1H); 4.62-4.55 (m, 1H); 4.50 (s, 2H); 4.18-4.08 (m, 1H); 2.04-1.90 (m, 1H); 1.87-1.60 (m, 4H); 1.60-1.44 (m, 1H).

### Example 42

### rac-2-Amino-6-({[2-(4-chlorophenyl)-1,3-thiazol-4-yl]methyl}thio)-4-[4-(tetrahydrofuran-3-yloxy)phenyl]pyridine-3,5-dicarbonitrile

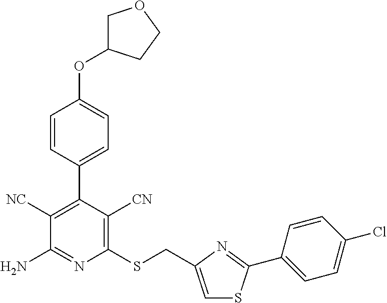

100 mg (0.30 mmol) of the compound of Example 24A and 79.4 mg (0.32 mmol) of 4-(chloromethyl)-2-(4-chlorophenyl)-1,3-thiazole were dissolved in 3.0 ml of DMF, 54.6 mg (0.65 mmol) of sodium bicarbonate were added and the mixture was stirred at 50° C. for 1 h. The reaction was then purified directly by preparative HPLC (Method 18).

Yield: 113 mg (70% of theory)

¹H-NMR (400 MHz, DMSO-d₆): δ=8.35-7.95 (br. s, 2H); 7.95 (d, 2H); 7.93 (s, 1H); 7.57 (d, 2H); 7.49 (d, 2H); 7.09 (d, 2H); 5.13 (br. t, 1H); 4.65 (s, 2H); 3.95-3.75 (m, 4H); 2.18 (m, 1H); 2.00 (m, 1H).

LC-MS (Method 7): Rₜ=2.40 min; MS (ESIpos): m/z=547 [M+H]⁺.

The compounds of the table below were prepared analogously to the procedure for Example 42 from Example 24A and the appropriate alkylation components:

| Example | Structure | Yield | Analysis |
|---|---|---|---|
| 43 | 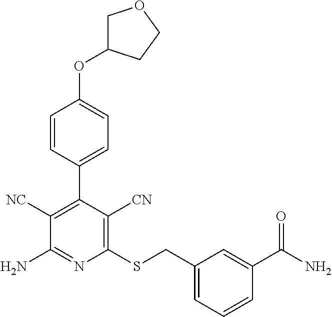 | 59% | LC-MS (Method 7): Rₜ = 1.64 min; MS (ESIpos): m/z = 472 [M + H]⁺ |
| 44 | 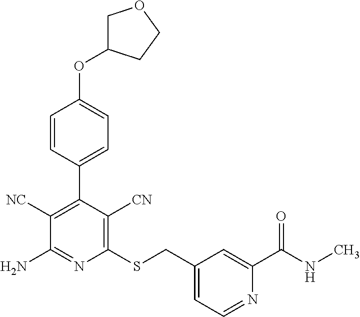 | 64% | LC-MS (Method 3): Rₜ = 2.29 min; MS (ESIpos): m/z = 487 [M + H]⁺ |
| 45 | 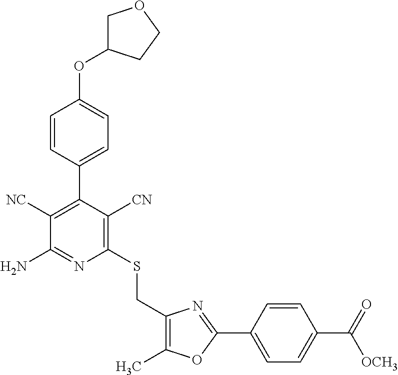 | 62% | LC-MS (Method 3): Rₜ = 2.28 min; MS (ESIpos): m/z = 568 [M + H]⁺ |
| 46 | 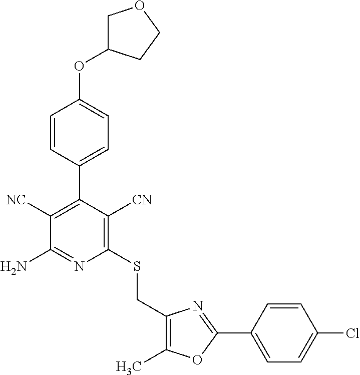 | 50% | LC-MS (Method 7): Rₜ = 2.40 min; MS (ESIpos): m/z = 545 [M + H]⁺ |
| 47 | 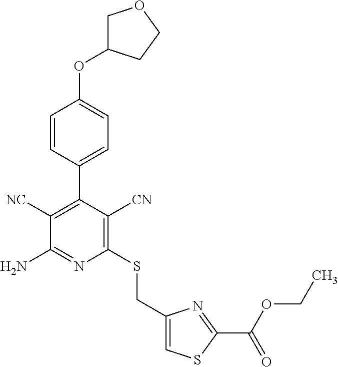 | 66% | LC-MS (Method 14): Rₜ = 1.20 min; MS (ESIpos): m/z = 508 [M + H]⁺ |
| 48 | 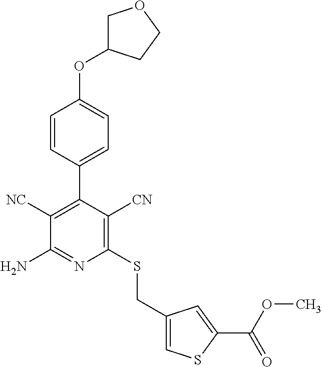 | 29% | LC-MS (Method 14): Rₜ = 1.24 min; MS (ESIpos): m/z = 493 [M + H]⁺ |

### Example 49

### rac-4-{4-[({6-Amino-3,5-dicyano-4-[4-(tetrahydrofuran-3-yloxy)phenyl]pyridin-2-yl}thio)methyl]-5-methyl-1,3-oxazol-2-yl}benzoic acid

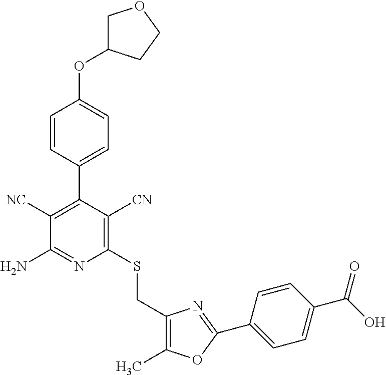

0.13 g (0.23 mmol) of the compound of Example 45 was suspended in 15 ml of dioxane, 0.46 ml (0.46 mmol) of 1 N aqueous sodium hydroxide solution was added and the mixture was stirred at 50° C. for 2 days. The pH was then adjusted to pH 3 using 1 N hydrochloric acid, and the mixture was diluted with 30 ml of water. The precipitate formed was filtered off, washed with water and dried.

Yield: 86 mg (68% of theory)

¹H-NMR (400 MHz, DMSO-d₆): δ=13.21 (s, 1H); 8.30-7.95 (br. m, 6H); 7.51 (d, 2H); 7.10 (d, 2H); 5.15 (br. t, 1H); 4.55 (s, 2H); 3.95-3.75 (m, 4H); 2.28 (m, 1H); 2.00 (m, 1H).

LC-MS (Method 7): Rₜ=1.88 min; MS (ESIpos): m/z=554 [M+H]⁺.

### Example 50

### rac-4-[({6-Amino-3,5-dicyano-4-[4-(tetrahydrofuran-3-yloxy)phenyl]pyridin-2-yl}sulfanyl)methyl]-1,3-thiazole-2-carboxamide

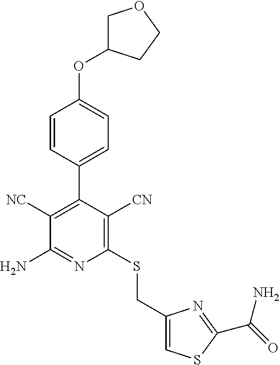

0.20 g (0.39 mmol) of the compound of Example 47 was dissolved in 4 ml methanol and 4 ml acetonitrile, 0.56 ml of a 7 M methanolic ammonia solution (3.94 mmol) was added and the mixture was stirred at 50° C. for 18 h. A further 5.0 ml of the ammonia solution were then added, and the mixture was stirred at room temperature for 2 h. The reaction was then freed from the solvent on a rotary evaporator, and the residue was purified by preparative HPLC (column: YMC GEL ODS-AQ S-5, 15 μm; mobile phase gradient: acetonitrile/water 10:90→95:5).

Yield: 105 mg (56% of theory)

¹H-NMR (400 MHz, DMSO-d₆): δ=8.35-7.95 (br. s, 1H); 8.13 (br. s, 1H); 8.11 (s, 1H); 7.85 (br. s, 1H); 7.48 (d, 2H); 7.09 (d, 2H); 5.11 (br. t, 1H); 4.60 (s, 2H); 3.95-3.75 (m, 4H); 2.27 (m, 1H); 2.00 (m, 1H).

LC-MS (Method 7): Rₜ=1.55 min; MS (ESIpos): m/z=478 [M+H]⁺.

### Example 51

### rac-4-[({6-Amino-3,5-dicyano-4-[4-(tetrahydrofuran-3-yloxy)phenyl]pyridin-2-yl}sulfanyl)methyl]-N-methyl-1,3-thiazole-2-carboxamide

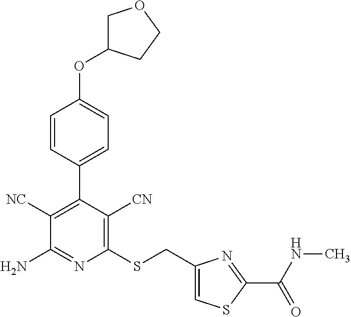

0.20 g (0.39 mmol) of the compound of Example 47 were dissolved in 10 ml of methanol and 5 ml of acetonitrile, 0.25 ml of a 33% strength ethanolic methylamine solution (1.97 mmol) was added and the mixture was stirred at room temperature for 18 h. The precipitate formed was filtered off, washed with a little methanol and dried.

Yield: 36 mg (19% of theory)

¹H-NMR (400 MHz, DMSO-d₆): δ=8.73 (q, 1H); 8.35-7.95 (br. s, 1H); 8.11 (s, 1H); 7.48 (d, 2H); 7.09 (d, 2H); 5.11 (br. t, 1H); 4.60 (s, 2H); 3.95-3.75 (m, 4H); 2.78 (d, 3H); 2.28 (m, 1H); 2.00 (m, 1H).

LC-MS (Method 7): Rₜ=1.69 min; MS (ESIpos): m/z=492 [M+H]⁺.

### Example 52

### rac-3-[({3,5-Dicyano-6-[(3R)-3-hydroxypyrrolidin-1-yl]-4-[4-(tetrahydrofuran-3-yloxy)phenyl]pyridin-2-yl}sulfanyl)methyl]benzamide

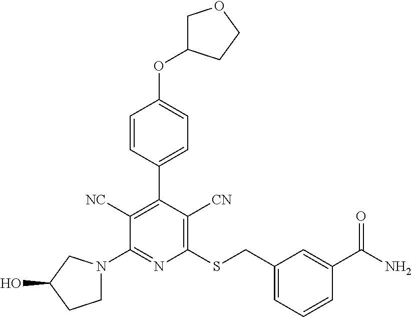

0.15 g (0.31 mmol) of the compound of Example 31A and 29.0 mg (0.33 mmol) of (R)-3-pyrrolidinol were stirred at room temperature in 3 ml of THF for 1 h. The reaction was then purified directly by preparative HPLC (Method 18).

Yield: 18.0 mg (11% of theory)

LC-MS (Method 3): Rₜ=2.09 min; MS (ESIpos): m/z=541 [M+H]⁺.

### Example 53

### rac-3-{[(3,5-Dicyano-6-{[(2S)-2,3-dihydroxypropyl]amino}-4-[4-(tetrahydrofuran-3-yloxy)phenyl]pyridin-2-yl)sulfanyl]methyl}benzamide

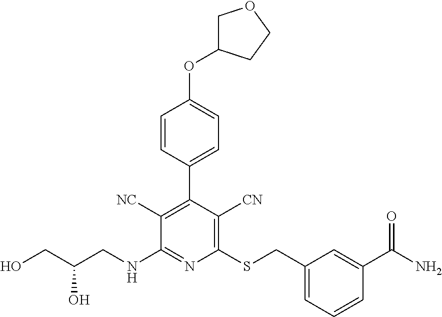

0.09 g (0.18 mmol) of the compound of Example 31A and 18.3 mg (0.20 mmol) of (S)-3-aminopropane-1,2-diol were stirred at room temperature in 1.8 ml of THF and 0.18 ml of DMSO for 1 h. The reaction was then purified directly by preparative HPLC (Method 18).

Yield: 54 mg (54% of theory)

LC-MS (Method 14): Rₜ=0.97 min; MS (ESIpos): m/z=545 [M+H]⁺.

### Example 54

### rac-2-Amino-6-({[2-(4-chlorophenyl)-1,3-thiazol-4-yl]methyl}sulfanyl)-4-[3-methoxy-4-(tetrahydrofuran-3-yloxy)phenyl]pyridine-3,5-dicarbonitrile

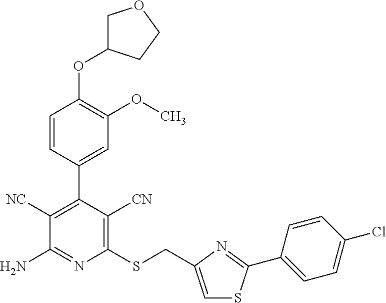

100 mg (46% pure, 0.12 mmol) of the compound of Example 25A and 30.5 mg (0.12 mmol) of 4-(chloromethyl)-2-(4-chlorophenyl)-1,3-thiazole were dissolved in 1.65 ml of DMF, 21.0 mg (0.25 mmol) of sodium bicarbonate were added and the mixture was stirred at 50° C. for 1 h. The reaction was then purified directly by preparative HPLC (column: YMC GEL ODS-AQ S-5, 15 μm; mobile phase gradient: acetonitrile/water 10:90→95:5).

Yield: 38 mg (53% of theory)

¹H-NMR (400 MHz, DMSO-d₆): δ=8.40-7.95 (br. s, 2H); 7.95 (d, 2H); 7.91 (s, 1H); 7.57 (d, 2H); 7.20 (d, 1H); 7.10 (m, 2H); 5.08 (br. m, 1H); 4.64 (s, 2H); 3.93-3.73 (m, 4H); 3.77 (s, 3H); 2.24 (m, 1H); 2.00 (m, 1H).

LC-MS (Method 3): Rₜ=2.83 min; MS (ESIpos): m/z=576 [M+H]⁺.

The compounds of the table below were prepared analogously to the procedure for Example 54:

| Example | Structure | Yield | Analysis |
|---|---|---|---|
| 55 | 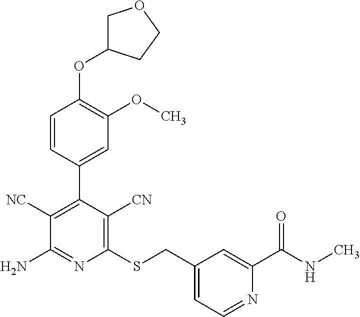 | 57% | LC-MS (Method 3): Rₜ = 2.09 min; MS (ESIpos): m/z = 516 [M + H]⁺ |
| 56 | 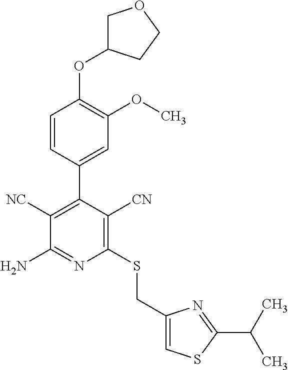 | 45% | LC-MS (Method 3): Rₜ = 2.45 min; MS (ESIpos): m/z = 507 [M + H]⁺ |

### Example 57

### rac-2-Amino-6-({[2-(4-chlorophenyl)-1,3-thiazol-4-yl]methyl}sulfanyl)-4-[3-fluoro-4-(tetrahydrofuran-3-yloxy)phenyl]pyridine-3,5-dicarbonitrile

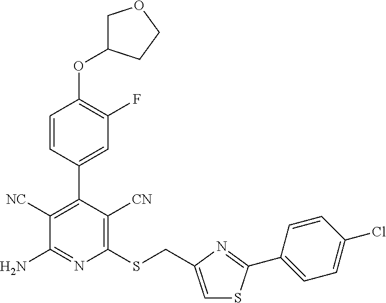

100 mg (93% pure, 0.26 mmol) of the compound of Example 26A and 31.5 mg (0.13 mmol) of 4-(chloromethyl)-2-(4-chlorophenyl)-1,3-thiazole were dissolved in 1.70 ml of DMF, 21.7 mg (0.26 mmol) of sodium bicarbonate were added and the mixture was stirred at 50° C. for 1 h. The reaction was then purified directly by preparative HPLC (column: YMC GEL ODS-AQ S-5, 15 μm; mobile phase gradient: acetonitrile/water 10:90→95:5).

Yield: 50 mg (34% of theory)

¹H-NMR (400 MHz, DMSO-d₆): δ=8.40-7.95 (br. s, 2H); 7.95 (d, 2H); 7.92 (s, 1H); 7.57 (d, 2H); 7.53 (d, 1H); 7.33 (m, 2H); 5.19 (br. m, 1H); 4.64 (s, 2H); 3.93 (dd, 1H); 3.96 (m, 2H); 3.77 (m, 1H); 2.29 (m, 1H); 2.02 (m, 1H).

LC-MS (Method 14): Rₜ=1.46 min; MS (ESIpos): m/z=564 [M+H]⁺.

The compound of the table below was prepared analogously to the procedure for Example 57:

| Example | Structure | Yield | Analysis |
|---|---|---|---|
| 58 | 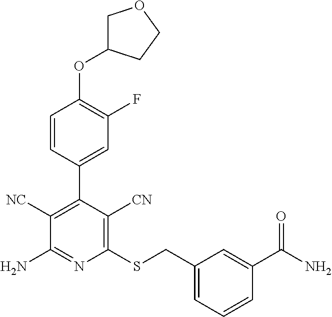 | 53% | LC-MS (Method 7): Rₜ = 1.68 min; MS (ESIpos): m/z = 490 [M + H]⁺ |

### Example 59

### rac-2-Amino-6-({[2-(4-chlorophenyl)-1,3-thiazol-4-yl]methyl}thio)-4-[4-(pyrrolidin-3-yloxy)phenyl]pyridine-3,5-dicarbonitrile

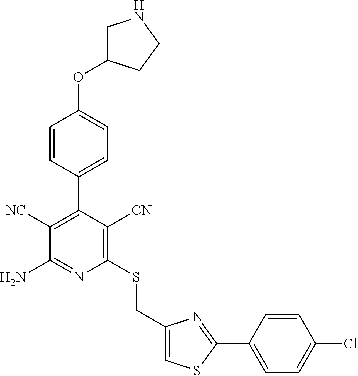

0.15 g (0.23 mmol) of the compound of Example 29A was suspended in 4.8 ml of dioxane, 1.5 ml of a 4 M solution of hydrogen chloride in dioxane were added and the mixture was stirred at room temperature for 6 h. The mixture was then stirred into 40 ml of a semiconcentrated aqueous solution of sodium bicarbonate. The precipitate was filtered off, washed with water and dried.

Yield: 86 mg (65% of theory)

¹H-NMR (400 MHz, DMSO-d₆): δ=8.35-7.95 (br. s, 2H); 7.95 (d, 2H); 7.92 (s, 1H); 7.57 (d, 2H); 7.46 (d, 2H); 7.05 (d, 2H); 4.92 (br. t, 1H); 4.63 (s, 2H); 3.08 (dd, 1H); 2.95-2.85 (m, 2H); 2.80-2.74 (m, 1H); 2.10-2.00 (m, 1H); 1.81-1.73 (m, 1H).

LC-MS (Method 7): Rₜ=1.49 min; MS (ESIpos): m/z=545 [M+H]⁺.

### Example 60

### rac-3-[({6-Amino-3,5-dicyano-4-[4-(tetrahydrofuran-3-yloxy)phenyl]pyridin-2-yl}oxy)methyl]benzoic acid

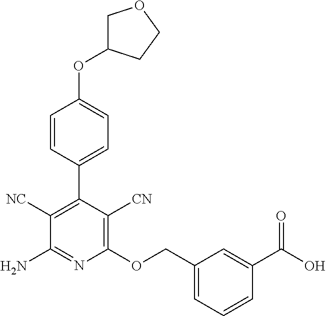

487 mg (4.34 mmol) of potassium tert-butoxide were suspended in 3.8 ml of 1,2-dimethoxyethane, 512 mg (2.9 mmol) of 3-(hydroxymethyl)benzoic acid and then 300 mg (0.724 mmol) of the compound of Example 41A were added and the mixture was then stirred at 60° C. for 12 h. 5 ml of water were then added, and the reaction solution was, with cooling, acidified with 1 N hydrochloric acid. The mixture was extracted three times with ethyl acetate, and the combined organic phases were washed once with saturated sodium chloride solution. After concentration by evaporation, the residue was purified by preparative HPLC (with addition of 0.1% TFA).

Yield: 113 mg (34% of theory)

¹H-NMR (400 MHz, DMSO-d₆): δ=13.08 (br. s, 1H); 8.40-7.65 (m, 5H); 7.55 (t, 1H); 7.49 (d, 2H); 7.09 (d, 2H); 5.52 (s, 2H); 5.13-5.10 (m, 1H); 3.98-3.72 (m, 4H); 2.32-2.20 (m, 1H); 2.06-1.95 (m, 1H).

LC-MS (Method 14): Rₜ=1.06 min; MS (ESIpos): m/z=457 [M+H]⁺.

### Example 61

### rac-3-[({6-Amino-3,5-dicyano-4-[4-(tetrahydrofuran-3-yloxy)phenyl]pyridin-2-yl}oxy)methyl]benzamide

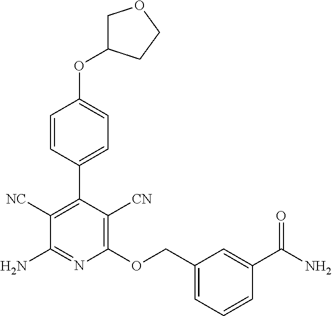

120 mg (0.237 mmol) of the compound of Example 60 were dissolved in 2 ml of DMF, 68 mg (0.355 mmol) of 1-(3-dimethylaminopropyl)-3-ethylcarbodiimide hydrochloride and 48 mg (0.355 mmol) of 1-hydroxy-1H-benzotriazole hydrate were added and the mixture was stirred at RT for 10 min. 63 mg (1.18 mmol) of ammonium chloride and 214 mg (0.36 mmol) of N,N-diisopropylethylamine were then added, and the mixture was stirred at RT overnight. Water was then added, and the reaction mixture was purified directly by preparative HPLC (with addition of 0.1% TFA).

Yield: 100 mg (93% of theory)

¹H-NMR (400 MHz, DMSO-d₆): δ=8.40-7.72 (m, 5H); 7.68 (d, 1H); 7.53-7.35 (m, 4H); 7.09 (d, 2H); 5.50 (s, 2H); 5.13-5.10 (m, 1H); 3.98-3.72 (m, 4H); 2.34-2.22 (m, 1H); 2.06-1.96 (m, 1H).

LC-MS (Method 7): Rₜ=1.54 min; MS (ESIpos): m/z=456 [M+H]⁺.

### Example 62

### Methyl 3-[1-({6-amino-3,5-dicyano-4-[4-(tetrahydrofuran-3-yloxy)phenyl]pyridin-2-yl}sulfanyl)ethyl]benzoate

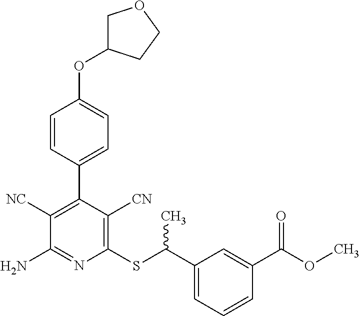

500 mg (1.478 mmol) of the compound of Example 45A were dissolved in 5 ml of DMF, 395 mg (1.625 mmol) of methyl 3-(1-bromoethyl)benzoate (for the preparation see, for example, U.S. Pat. No. 4,499,299) and 372 mg (4.433 mmol) of sodium bicarbonate were added and the mixture was stirred at RT for 3 hours. Water was then added in such an amount that a clear solution was formed. The reaction mixture was then purified directly by preparative HPLC (with addition of 0.1% TFA).

Yield: 577 mg (78% of theory)

¹H-NMR (400 MHz, DMSO-d₆): δ=8.30-7.95 (m, 3H); 7.90 (d, 1H); 7.86 (d, 1H); 7.51 (d, 1H); 7.46 (d, 2H); 7.07 (d, 2H); 5.28 (q, 1H); 5.11 (m, 1H); 3.92 (dd, 1H); 3.87 (s, 3H); 3.84-3.74 (m, 3H); 2.27 (m, 1H); 1.99 (m, 1H); 1.75 (d, 3H).

LC-MS (Method 7): Rₜ=2.18 min; MS (ESIpos): m/z=501 [M+H]⁺.

### Example 63 and Example 64

### Methyl ent-3-[1-({6-amino-3,5-dicyano-4-[4-(tetrahydrofuran-3-yloxy)phenyl]pyridin-2-yl}sulfanyl)ethyl]benzoate

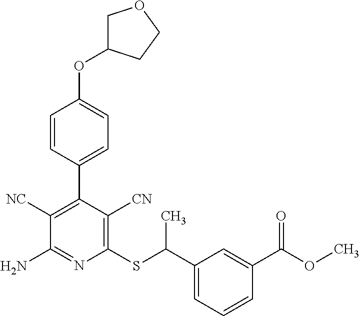

By preparative HPLC on a chiral phase, the compound from Example 62 (530 mg) was separated into the enantiomers [column: Daicel Chiralcel OD-H, 5 μm, 250 mm×20 mm; mobile phase: isohexane/isopropanol (1:1); flow rate: 15 ml/min; temperature: 40° C.; UV detection: 220 nm]:

### Example 63

### Enantiomer 1

Yield: 258 mg (chem. purity >99%, >99% ee)

Rₜ=6.16 min.

[column: Daicel Chiralcel OD-H, 5 μm, 250 mm×4.6 mm; mobile phase: isohexane/isopropanol (4:6); flow rate: 1.0 ml/min; temperature: 40° C.; UV detection: 215 nm].

### Example 64

### Enantiomer 2

Yield: 248 mg (chem. purity >99%, >99% ee)

Rₜ=8.62 min.

[column: Daicel Chiralcel OD-H, 5 μm, 250 mm×4.6 mm; mobile phase: isohexane/isopropanol (4:6); flow rate: 1.0 ml/min; temperature: 40° C.; UV detection: 215 nm].

### Example 65

### ent-3-[1-({6-Amino-3,5-dicyano-4-[4-(tetrahydrofuran-3-yloxy)phenyl]pyridin-2-yl}-sulfanyl)ethyl]benzoic acid

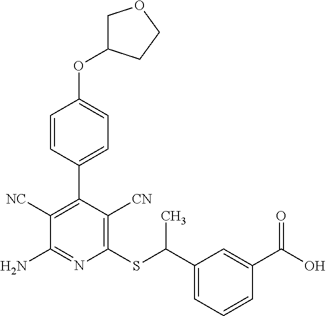

245 mg (0.489 mmol) of the compound of Example 64 were dissolved in 10 ml of THF, 0.98 ml of a 1 N solution of lithium hydroxide in water was added and the mixture was stirred at 40° C. overnight. After cooling, the reaction mixture was acidified with 1 N hydrochloric acid and the solution was purified directly by preparative HPLC (with addition of 0.1% TFA).

Yield: 234 mg (98% of theory)

¹H-NMR (400 MHz, DMSO-d₆): δ=13.08 (br. s, 1H); 8.40-7.78 (m, 5H); 7.49-7.42 (m, 3H); 7.09 (d, 2H); 5.30 (q, 1H); 5.12-5.09 (m, 1H); 3.95-3.71 (m, 4H); 2.31-2.20 (m, 1H); 2.05-1.95 (m, 1H); 1.76 (d, 3H).

LC-MS (Method 3): Rₜ=2.34 min; MS (ESIpos): m/z=487 [M+H]⁺.

### Example 66

### ent-3-[1-({6-Amino-3,5-dicyano-4-[4-(tetrahydrofuran-3-yloxy)phenyl]pyridin-2-yl}-sulfanyl)ethyl]benzamide

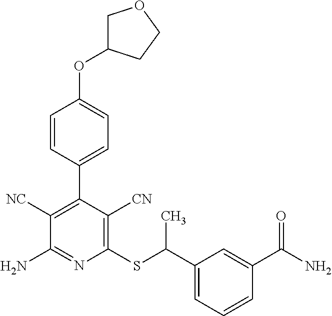

75 mg (0.154 mmol) of the compound of Example 65 were dissolved in 1.5 ml of DMF, 44 mg (0.231 mmol) of 1-(3-dimethylaminopropyl)-3-ethylcarbodiimide hydrochloride and 31 mg (0.231 mmol) of 1-hydroxy-1H-benzotriazole hydrate were added and the mixture was stirred at RT for 10 minutes. 41 mg (0.771 mmol) of ammonium chloride and 139 mg (1.079 mmol) of N,N-diisopropylethylamine were then added, and the mixture was stirred at RT overnight. Water was then added, and the reaction mixture was purified directly by preparative HPLC (with addition of 0.1% TFA).

Yield: 47 mg (63% of theory)

¹H-NMR (400 MHz, DMSO-d₆): δ=8.40-7.82 (m, 4H); 7.79 (d, 1H); 7.75 (d, 1H); 7.49-7.38 (m, 4H); 7.08 (d, 2H); 5.21 (q, 1H); 5.12-5.09 (m, 1H); 3.94-3.71 (m, 4H); 2.32-2.20 (m, 1H); 2.04-1.95 (m, 1H); 1.75 (d, 3H).

LC-MS (Method 20): Rₜ=2.06 min; MS (ESIpos): m/z=486 [M+H]⁺.

### B. ASSESSING THE PHARMACOLOGICAL AND PHYSIOLOGICAL ACTIVITY

The pharmacological and physiological activity of the compounds according to the invention can be demonstrated in the following assays:

### B-1. Indirect Determination of the Adenosine Agonism by Way of Gene Expression

Cells of the CHO (Chinese Hamster Ovary) permanent cell line are transfected stably with the cDNA for the adenosine receptor subtypes A1, A2a and A2b. The adenosine A1 receptors are coupled to the adenylate cyclase by way of Gᵢ proteins, while the adenosine A2a and A2b receptors are coupled by way of Gₛ proteins. In correspondence with this, the formation of cAMP in the cell is inhibited or stimulated, respectively. After that, expression of the luciferase is modulated by way of a cAMP-dependent promoter. The luciferase test is optimized, with the aim of high sensitivity and reproducibility, low variance and good suitability for implementation on a robot system, by varying several test parameters, such as cell density, duration of the growth phase and the test incubation, forskolin concentration and medium composition. The following test protocol is used for pharmacologically characterizing cells and for the robot-assisted substance screening:

The stock cultures are grown, at 37° C. and under 5% CO₂, in DMEM/F12 medium containing 10% FCS (fetal calf serum) and in each case split 1:10 after 2-3 days. The test cultures are seeded in 384-well plates with 2000 cells per well and grown at 37° C. for approx. 48 hours. The medium is then replaced with a physiological sodium chloride solution (130 mM sodium chloride, 5 mM potassium chloride, 2 mM calcium chloride, 20 mM HEPES, 1 mM magnesium chloride hexahydrate, 5 mM sodium bicarbonate, pH 7.4). The substances to be tested, which are dissolved in DMSO, are pipetted into the test cultures (maximum final concentration of DMSO in the test mixture: 0.5%) in a dilution series of from 5×10⁻¹¹M to 3×10⁻⁶M (final concentration). 10 minutes later, forskolin is added to the A1 cells and all the cultures are subsequently incubated at 37° C. for four hours. After that, 35 μl of a solution which is composed of 50% lysis reagent (30 mM disodium hydrogenphosphate, 10% glycerol, 3% TritonX100, 25 mM TrisHCl, 2 mM dithiotreitol (DTT), pH 7.8) and 50% luciferase substrate solution (2.5 mM ATP, 0.5 mM luciferin, 0.1 mM coenzyme A, 10 mM tricine, 1.35 mM magnesium sulfate, 15 mM DTT, pH 7.8) are added to the test cultures, which are shaken for approx. 1 minute and the luciferase activity is measured using a camera system. The EC₅₀ values are determined, i.e., the concentrations at which 50% of the luciferase answer is inhibited in the case of the A1 cell, and, respectively, 50% of the maximum stimulation with the corresponding substance is achieved in the case of the A2b and A2a cells. The adenosine-analogous compound NECA (5-N-ethylcarboxamidoadenosine), which binds to all adenosine receptor subtypes with high affinity and possesses an agonistic effect, is used in these experiments as the reference compound [Klotz, K. N., Hessling, J., Hegler, J., Owman, C., Kull, B., Fredholm, B. B., Lohse, M. J., “Comparative pharmacology of human adenosine receptor subtypes—characterization of stably transfected receptors in CHO cells”, Naunyn Schmiedebergs Arch. Pharmacol., 357 (1998), 1-9].

Table 1 below lists the EC₅₀ values of representative working examples for the receptor stimulation on adenosine A1, A2a and A2b receptor subtypes:

TABLE 1

| Example | EC₅₀ A1 [nM] | EC₅₀ A2a | EC₅₀ A2b |
|---|---|---|---|
| No. | (1 μM forskolin) | [nM] | [nM] |
| 8 | 6.9 | 497 | 2.4 |
| 9 | 104 | 3000 | 3.9 |
| 10 | 265 | 3000 | 4.3 |
| 14 | 56 | 1920 | 16 |
| 23 | 168 | 518 | 6.9 |
| 24 | 0.14 | 506 | 3000 |
| 29 | 0.18 | 1270 | 3000 |
| 32 | 0.86 | 144 | 720 |
| 35 | 0.56 | 874 | 43 |
| 41 | 2.9 | 480 | 11 |
| 43 | 0.16 | 424 | 537 |
| 44 | 0.08 | 69 | 234 |
| 49 | 0.22 | 782 | 3000 |
| 50 | 0.78 | 1100 | 556 |
| 51 | 0.51 | 261 | 177 |
| 58 | 0.63 | 909 | 1010 |
| 64 | 4.1 | 3000 | 3000 |
| 66 | 0.53 | 3000 | 1780 |

### B-2. Studies on Isolated Blood Vessels

The caudal artery of anesthetized rats is excised and mounted in a conventional apparatus for measuring isolated blood vessels. The vessels are perfused in a heated bath and contracted using phenylephrine. The extent of the contraction is determined using a contraction meter. Test substances are added to the precontracted blood vessels, and the reduction of the contraction of the vessels is measured. A reduction of contraction corresponds to a dilation of the vessels. The concentration at which the contraction of the blood vessels is reduced by 50% is given as the EC₅₀ value of a test substance with respect to its relaxing properties.

### B-3. Measurement of Blood Pressure and Heart Rate on Awake Rats

Various dosages of test substances are administered orally to awake SHR rats (spontaneously hypertensive rats) carrying an internal transmitter capable of measuring permanently both blood pressure and heart rate (telemetric monitoring of hemodynamic parameters). Blood pressure, heart rate and their changes are then recorded over a period of 24 hours.

### B-4. Measurement of Blood Pressure and Heart Rate on Awake Marmosets

Various concentrations of the test substances are administered orally to awake marmosets which carry an internal transmitter capable of measuring permanently both blood pressure and heart rate (telemetric monitoring of hemodynamic parameters). Blood pressure, heart rate and their changes are then recorded for a period of 6-24 hours.

B-5. Determination of Pharmacokinetic Parameters after Intravenous and Oral Administration

The substance to be tested is administered intravenously as a solution to animals (for example mice, rats, dogs), and oral administration takes place as solution or suspension by gavage. After administration of the substance, blood is taken from the animals at fixed times and is heparinized, and then plasma is obtained therefrom by centrifugation. The substance is quantified analytically in the plasma by LC/MS-MS. The plasma concentration/time courses found in this way are used to calculate the pharmacokinetic parameters such as AUC area under the concentration/time curve), Cₘₐₓ (maximum plasma concentration), T_{1/2} (half-life) and CL (clearance) by means of a validated pharmacokinetic computer program.

### B-6. Determination of the Solubility

### Reagents Required:

PBS buffer pH 6.5: 90.00 g of NaCl p.a. (for example from Merck, Art. No. 1.06404.1000), 13.61 g of KH₂PO₄ p.a. (for example from Merck, Art. No. 1.04873.1000) and 83.35 g of 1 N aqueous sodium hydroxide solution (for example from Bernd Kraft GmbH, Art. No. 01030.4000) are weighed out into a 1 iter measuring flask and made up with distilled water to 1 iter, and the mixture is stirred for 1 hour. Using 1 N hydrochloric acid (for example from Merck, Art. No. 1.09057.1000), the pH is then adjusted to 6.5.

PEG/water solution (70:30 v/v): 70 ml of polyethylene glycol 400 (for example from Merck, Art. No. 8.17003.1000) and 30 ml of distilled water are homogenized in a 100 ml measuring flask.

PEG/PBS buffer pH 6.5 (20:80 v/v): 20 ml of polyethylene glycol 400 (for example from Merck, Art. No. 8.17003.1000) and 80 ml of PBS buffer pH 6.5 are homogenized in a 100 ml measuring flask.

dimethyl sulfoxide (for example from Baker, Art. No. 7157.2500)

distilled water.

### Preparation of the Starting Solution (Original Solution):

At least 4 mg of the test substance are weighed out accurately into a wide mouth 10 mm Screw V-Vial (from Glastechnik Grafenroda GmbH, Art. No. 8004-WM-H/V15μ) with fitting screw cap and septum, DMSO is added with a pipetting robot to give a concentration of 50 mg/ml and the mixture is shaken for 10 minutes.

### Preparation of the Calibration Solutions:

Preparation of the Starting Solution for Calibration Solutions (Stock Solution): with the aid of a pipetting robot, 10 μl of the original solution are transferred into a microtiter plate, and DMSO is added to give a concentration of 600 μg/ml. The sample is shaken until it is dissolved completely.

Calibration solution 1 (20 μg/ml): 1000 μl of DMSO are added to 34.4 μl of the stock solution, and the mixture is homogenized.

Calibration solution 2 (2.5 μg/ml): 700 μl of DMSO are added to 100 μl of calibration solution 1, and the mixture is homogenized.

### Preparation of the Sample Solutions:

Sample solution for solubilities of up to 5 g/liter in PBS buffer pH 6.5: 10 μl of the original solution are transferred into a microtiter plate, and 1000 μl of PBS buffer pH 6.5 are added.

Sample solution for solubilities of up to 5 g/liter in PEG/water (70:30): 10 μl of the original solution are transferred into a microtiter plate, and 1000 μl of PEG/water (70:30) are added.

Sample solution for solubilities of up to 5 g/liter in PEG/PBS buffer pH 6.5 (20:80): 10 μl of the original solution are transferred into a microtiter plate, and 1000 μl of PEG/PBS buffer pH 6.5 (20:80) are added.

### Procedure:

Using a temperature-adjustable shaker (e.g. from Eppendorf Thermomixer comfort Art. No. 5355 000.011 with Thermoblock Art. No. 5362.000.019), the sample solutions prepared in this manner are shaken at 20° C. and 1400 rpm for 24 hours. From these solutions, in each case 180 μl are removed and transferred into Beckman polyallomer centrifuge tubes (Art. No. 343621). These solutions are centrifuged at about 223 000×g for 1 hour (e.g. from Beckman Optima L-90K Ultracentrifuge with type 42.2 Ti rotor at 42 000 rpm). From each sample solution, 100 μl of the supernatant are removed and diluted 1:5 and 1:100 with DMSO. From each dilution, a sample is removed into a suitable vessel for HPLC analysis.

### Analysis:

The samples are analyzed by RP-HPLC. A two-point calibration plot of the test compound in DMSO is used for quantification. The solubility is expressed in mg/l. Analysis sequence: 1) calibration solution 2.5 mg/ml; 2) calibration solution 20 μg/ml; 3) sample solution 1:5; 4) sample solution 1:100.

### HPLC Method for Acids:

Agilent 1100 with DAD (G1315A), quat. pump (G1311A), autosampler CTC HTS PAL, degasser (G1322A) and column thermostat (G1316A); column: Phenomenex Gemini C18, 50 mm×2 mm, 5μ; temperature: 40° C.; eluent A: water/phosphoric acid pH 2; eluent B: acetonitrile; flow rate: 0.7 ml/min; gradient: 0-0.5 min 85% A, 15% B; ramp: 0.5-3 min 10% A, 90% B; 3-3.5 min 10% A, 90% B; ramp: 3.5-4 min 85% A, 15% B; 4-5 min 85% A, 15% B.

### HPLC Method for Bases:

Agilent 1100 with DAD (G1315A), quat. pump (G1311A), autosampler CTC HTS PAL, degasser (G1322A) and column thermostat (G1316A); column: VDSoptilab Kromasil 100 C18, 60 mm×2.1 mm, 3.5μ; temperature: 30° C.; eluent A: water+5 ml perchloric acid/l; eluent B: acetonitrile; flow rate: 0.75 ml/min; gradient: 0-0.5 min 98% A, 2% B; ramp: 0.5-4.5 min 10% A, 90% B; 4.5-6 min 10% A, 90% B; ramp: 6.5-6.7 min 98% A, 2% B; 6.7-7.5 min 98% A, 2% B.

### C. WORKING EXAMPLES OF PHARMACEUTICAL COMPOSITIONS

The compounds of the invention can be converted into pharmaceutical preparations in the following ways:

### Tablet:

### Composition:

100 mg of the compound of the invention, 50 mg of lactose (monohydrate), 50 mg of maize starch (native), 10 mg of polyvinylpyrrolidone (PVP 25) (from BASF, Ludwigshafen, Germany) and 2 mg of magnesium stearate.

Tablet weight 212 mg, diameter 8 mm, radius of curvature 12 mm.

### Production:

The mixture of compound of the invention, lactose and starch is granulated with a 5% strength solution (m/m) of the PVP in water. The granules are dried and mixed with the magnesium stearate for 5 minutes. This mixture is compressed in a conventional tablet press (see above for format of the tablet). A guideline compressive force for the compression is 15 kN.

Suspension which can be Administered Orally:

### Composition:

1000 mg of the compound of the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound of the invention.

### Production:

The Rhodigel is suspended in ethanol, and the compound of the invention is added to the suspension. The water is added while stirring. The mixture is stirred for about 6 h until the swelling of the Rhodigel is complete.

Solution which can be Administered Orally:

### Composition:

500 mg of the compound of the invention, 2.5 g of polysorbate and 97 g of polyethylene glycol 400.20 g of oral solution correspond to a single dose of 100 mg of the compound of the invention.

### Production:

The compound of the invention is suspended in the mixture of polyethylene glycol and polysorbate with stirring. The stirring process is continued until the compound of the invention has completely dissolved.

i.v. solution:

The compound of the invention is dissolved in a concentration below the saturation solubility in a physiologically tolerated solvent (e.g. isotonic saline, 5% glucose solution and/or 30% PEG 400 solution). The solution is sterilized by filtration and used to fill sterile and pyrogen-free injection containers.

1. A compound of the formula (I) 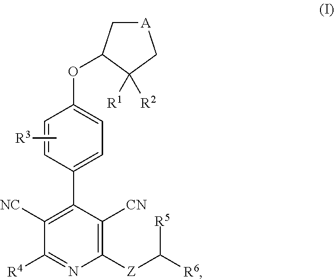
in which
A represents CH₂, CH₂CH₂, O, N—R⁷, S, S(═O) or S(═O)₂ in which
R⁷ represents hydrogen, (C₁-C₄)-alkyl, (C₁-C₄)-acyl or (C₁-C₄)-alkylsulfonyl,
where the alkyl, acyl and alkylsulfonyl groups mentioned for their part may be substituted by hydroxyl, amino or carboxyl,
Z represents O or S,
R¹ represents hydrogen,
R² represents hydrogen, hydroxyl, amino, mono-(C₁-C₄)-alkylamino or di-(C₁-C₄)-alkylamino
or
R¹ and R² together with the carbon atom to which they are attached form a carbonyl group,
R³ represents hydrogen, halogen, cyano, (C₁-C₄)-alkyl or (C₁-C₄)-alkoxy,
where the alkyl and alkoxy groups mentioned may be substituted up to three times by fluorine,
R⁴ represents a group of the formula —OR⁸ or —NR⁹R¹⁰ in which
R⁸ represents (C₁-C₆)-alkyl which may be mono- or disubstituted by identical or different substituents from the group consisting of hydroxyl, (C₁-C₄)-alkoxy, carboxyl and (C₁-C₄)-alkoxycarbonyl or may be substituted up to three times by fluorine, or represents (C₄-C₆)-cycloalkyl,
and
R⁹ and R¹⁰ are identical or different and independently of one another represent hydrogen or (C₁-C₆)-alkyl which may be substituted up to three times by fluorine or mono- or disubstituted by identical or different substituents from the group consisting of hydroxyl, (C₁-C₄)-alkoxy, amino, mono-(C₁-C₄)-alkylamino, di-(C₁-C₄)-alkylamino, carboxyl, (C₁-C₄)-alkoxycarbonyl and a 4- to 7-membered heterocycle,
where the heterocycle mentioned contains one or two ring heteroatoms from the group consisting of N, O and S and for its part may be mono- or disubstituted by identical or different substituents from the group consisting of (C₁-C₄)-alkyl, hydroxyl, oxo and (C₁-C₄)-alkoxy,
or
R⁹ and R¹⁰ together with the nitrogen atom to which they are attached form a 4- to 7-membered heterocycle which may contain a further ring heteroatom from the group consisting of N, O and S and may be mono- or disubstituted by identical or different substituents from the group consisting of fluorine, (C₁-C₄)-alkyl, hydroxyl, oxo, (C₁-C₄)-alkoxy, amino, mono-(C₁-C₄)-alkylamino, di-(C₁-C₄)-alkylamino, azetidino, pyrrolidino, piperidino and morpholino,
R⁵ represents hydrogen or (C₁-C₄)-alkyl
and
R⁶ represents (C₁-C₄)-alkyl which may be substituted by hydroxyl, (C₁-C₄)-alkoxy or up to three times by fluorine
or
represents (C₆-C₁₀)-aryl or 5- to 10-membered heteroaryl having up to three ring heteroatoms from the group consisting of N, O and S, each of which cycles may be
(i) mono- or disubstituted by identical or different radicals from the group consisting of halogen, nitro, cyano, (C₁-C₆)-alkyl, trifluoromethyl, hydroxyl, (C₁-C₆)-alkoxy, amino, mono-(C₁-C₆)-alkylamino, di-(C₁-C₆)-alkylamino, (C₁-C₆)-acylamino, (C₁-C₆)-alkylsulfonylamino, carboxyl, (C₁-C₆)-alkoxycarbonyl, aminocarbonyl, mono-(C₁-C₆)-alkylaminocarbonyl, di-(C₁-C₆)-alkylaminocarbonyl, aminosulfonyl, mono-(C₁-C₆)-alkylaminosulfonyl and di-(C₁-C₆)-alkylaminosulfonyl
and/or
(ii) substituted by pyrrolidino, piperidino, morpholino, piperazino, N′-(C₁-C₄)-alkylpiperazino, tetrazolyl or a group of the formula -L-R¹¹ in which
L represents a bond, NH or O
and
R¹¹ represents phenyl or 5- or 6-membered heteroaryl having up to three ring heteroatoms from the group consisting of N, O and S, each of which cycles may be mono- to trisubstituted by identical or different radicals from the group consisting of halogen, nitro, cyano, (C₁-C₆)-alkyl, trifluoromethyl, hydroxyl, (C₁-C₆)-alkoxy, difluoromethoxy, trifluoromethoxy, amino, mono-(C₁-C₆)-alkylamino, di-(C₁-C₆)-alkylamino, (C₁-C₆)-alkoxycarbonyl and carboxyl,
or an N-oxide or salt thereof.

2. The compound of the formula (I) as claimed in claim 1 in which
A represents CH₂, CH₂CH₂, O or NH,
Z represents O or S,
R¹ represents hydrogen,
R² represents hydrogen, hydroxyl or amino,
R³ represents hydrogen, fluorine, chlorine, methyl or methoxy,
R⁴ represents a group of the formula —NR⁹R¹⁰ in which
R⁹ represents hydrogen,
R¹⁰ represents hydrogen or (C₁-C₄)-alkyl which may be mono- or disubstituted by identical or different substituents from the group consisting of hydroxyl, (C₁-C₄)-alkoxy, amino, mono-(C₁-C₄)-alkylamino and di-(C₁-C₄)-alkylamino
or
R⁹ and R¹⁰ together with the nitrogen atom to which they are attached form a 4- to 6-membered heterocycle which may contain a further ring heteroatom from the group consisting of N and O and may be mono- or disubstituted by identical or different substituents from the group consisting of (C₁-C₄)-alkyl, hydroxyl, (C₁-C₄)-alkoxy, amino, mono-(C₁-C₄)-alkylamino and di-(C₁-C₄)-alkylamino,
R⁵ represents hydrogen or methyl
and
R⁶ represents phenyl or 5- or 6-membered heteroaryl having up to two ring heteroatoms from the group consisting of N, O and S, each of which cycles may be
(i) mono- or disubstituted by identical or different radicals from the group consisting of fluorine, chlorine, cyano, (C₁-C₄)-alkyl, trifluoromethyl, (C₁-C₄)-alkoxy, amino, carboxyl, (C₁-C₄)-alkoxycarbonyl, aminocarbonyl and mono-(C₁-C₄)-alkylaminocarbonyl
and/or
(ii) substituted by a group of the formula -L-R¹¹ in which
L represents a bond or NH
and
R¹¹ represents phenyl or pyridyl, each of which may be mono- or disubstituted by identical or different radicals from the group consisting of fluorine, chlorine, cyano, (C₁-C₄)-alkyl, trifluoromethyl, (C₁-C₄)-alkoxy, trifluoromethoxy, (C₁-C₄)-alkoxycarbonyl and carboxy.

3. The compound of the formula (I) as claimed in claim 1 in which
A represents CH₂ or O,
Z represents S,
R¹ represents hydrogen,
R² represents hydrogen or hydroxyl,
R³ represents hydrogen or fluorine,
R⁴ represents a group of the formula —NR⁹R¹⁰ in which
R⁹ represents hydrogen,
R¹⁰ represents hydrogen or (C₁-C₄)-alkyl which may be mono- or disubstituted by hydroxyl
or
R⁹ and R¹⁰ together with the nitrogen atom to which they are attached form an azetidino, pyrrolidino or piperidino ring, each of which may be substituted by hydroxyl,
R⁵ represents hydrogen
and
R⁶ represents phenyl, pyridyl, oxazolyl or thiazolyl, each of which may be
(i) mono- or disubstituted by identical or different radicals from the group consisting of fluorine, chlorine, cyano, methyl, trifluoromethyl, amino, carboxyl, methoxycarbonyl, ethoxycarbonyl, aminocarbonyl and methylaminocarbonyl
and/or
(ii) substituted by a group of the formula -L-R¹¹ in which
L represents a bond or NH
and
R¹¹ represents phenyl which may be mono- or disubstituted by identical or different radicals from the group consisting of fluorine, chlorine, methyl, trifluoromethyl, methoxycarbonyl, ethoxycarbonyl and carboxyl.

4. A process for preparing the compounds of the formula (I) as defined in claim 1 and in which R⁴ represents NH₂, characterized in that
[A] a compound of the formula (II) 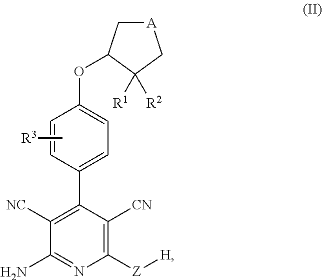
in which A, R¹, R², R³ and Z each have the meanings given in claim 1, is reacted in an inert solvent in the presence of a base with a compound of the formula (III) 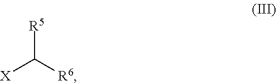
in which R⁵ and R⁶ have the meanings given in claim 1 and
X represents a suitable leaving group, preferably halogen, in particular chlorine, bromine or iodine, or represents mesylate, tosylate or triflate,
or alternatively, if Z represents O,
[B] a compound of the formula (IV-A) 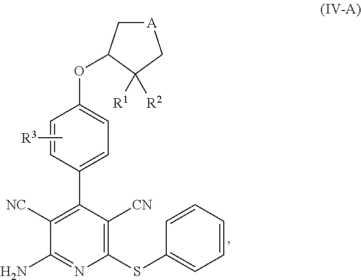
in which A, R¹, R² and R³ each have the meanings given in claim 1, is reacted in an inert solvent in the presence of a base with a compound of the formula (V) 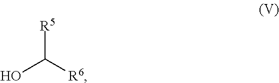
in which R⁵ and R⁶ have the meanings given in claim 1,
and the resulting compounds of the formula (I-A) 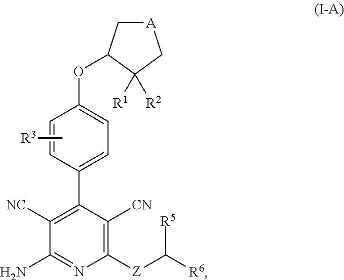
in which A, R¹, R², R³, R⁵, R⁶ and Z each have the meanings given in claim 1, are, if appropriate, separated into their enantiomers and/or diastereomers and/or, if appropriate, converted with the appropriate (i) solvents and/or (ii) bases or acids into their salts.

5-7. (canceled)

8. A medicament comprising a compound of the formula (I) as defined in claim 1 in combination with one or more inert nontoxic pharmaceutically suitable auxiliaries.

9. A medicament comprising a compound of the formula (I) as defined in claim 1 in combination with one or more further active compounds selected from the group consisting of lipid metabolism-modifying active compounds, antidiabetics, antihypertensive drugs and antithrombotic drugs.

10. (canceled)

11. A method for the treatment and/or prophylaxis of hypertension, coronary heart disease, acute coronary syndrome, angina pectoris, heart failure, myocardial infarction and atrial fibrillation, diabetes, metabolic syndrome and dyslipidemias in humans and animals comprising the step of administering to a human or animal in need thereof an effective amount of at least one compound of the formula (I) as defined in claim 1.
